(19) United States
(12) Patent Application Publication
(10) Pub. No.: US 2025/0281392 A1
(43) Pub. Date: Sep. 11, 2025

(54) TOPICAL DELIVERY OF EPINEPHRINE AND PRODRUG COMPOSITIONS

(71) Applicant: Aquestive Therapeutics, Inc., Warren, NJ (US)

(72) Inventors: Stephen Paul Wargacki, Pittstown, NJ (US); Alexander Mark Schobel, Vero Beach, FL (US); Carl Neil Kraus, Raleigh, NC (US); Vincent Buono, Basking Ridge, NJ (US); Michael Koons, York, PA (US); Ami Patel, Warren, NJ (US)

(73) Assignee: Aquestive Therapeutics, Inc., Warren, NJ (US)

(21) Appl. No.: 19/074,199

(22) Filed: Mar. 7, 2025

Related U.S. Application Data
(60) Provisional application No. 63/563,287, filed on Mar. 8, 2024.
(60) Provisional application No. 63/669,942, filed on Jul. 11, 2024.
(60) Provisional application No. 63/698,456, filed on Sep. 24, 2024.

(51) Int. Cl. A61K 9/00; A61K 9/06; A61K 31/222; A61K 47/10; A61K 47/36; A61K 47/38; A61P 37/06

(52) U.S. Cl. CPC A61K 9/0014; A61K 9/06; A61K 31/222; A61K 47/10; A61K 47/36; A61K 47/38; A61P 37/06

(57) ABSTRACT

Pharmaceutical compositions including topical compositions of prodrugs are described, including prodrugs of epinephrine, which can be used to treat conditions relating to allergy, dermatology and immunology and the compositions having enhanced active component permeation properties are described.

### TECHNICAL FIELD

This invention relates to pharmaceutical compositions. The application claims priority to U.S. Provisional Patent Application No. 63/563,287, filed Mar. 8, 2024, U.S. Provisional Patent Application No. 63/669,942, filed Jul. 11, 2024, and U.S. Provisional Patent Application No. 63/698,456, filed Sep. 24, 2024, each of which is incorporated in its entirety.

### BACKGROUND

A pharmaceutical composition with enhanced stability delivered to patients in a topical formulation. Delivery of drugs or pharmaceuticals using composition transdermally or transmucosally can require that the drug or pharmaceutical permeate or otherwise cross a biological membrane in an effective and efficient manner.

### SUMMARY

In one aspect, a topical pharmaceutical composition can include a pharmaceutically active component including at least one prodrug of epinephrine and a skin permeability enhancer.

In another aspect, a method of making a topical pharmaceutical composition can include combining a pharmaceutically active component including at least one prodrug of epinephrine and a skin permeability enhancer, and forming a pharmaceutical composition including the skin permeability enhancer and the pharmaceutically active component.

In certain embodiments, the pharmaceutical composition includes a gelling polymer. In certain embodiments, the gelling polymer is a non-ionic cellulosic polymer. In certain embodiments, the pharmaceutical composition includes gelling polymer is a cross-linked polyacrylic acid. In certain embodiments, the pharmaceutical composition includes a solvent. In certain embodiments, the pharmaceutical composition includes spreadability agent. In certain embodiments, the pharmaceutical composition includes antioxidant. In certain embodiments, the pharmaceutical composition includes a preservative. In certain embodiments, the pharmaceutical composition includes a pH modifier. In certain embodiments, the pharmaceutical composition includes a viscosity agent. In certain embodiments, the pharmaceutical composition includes the gelling polymer is a arabinogalactan or gum arabic polymer.

In another aspect, a method of treating a medical condition can include administering an effective amount of a topical pharmaceutical composition including a pharmaceutically active component including at least one prodrug of epinephrine and a skin permeability enhancer.

In certain circumstances, the composition can include a permeation enhancer.

In certain circumstances, the topical composition can include formed from a gauze, hydrogel, ampule, solution, paste, a cream, a lotion, a powder, emulsion, an ointment, a gel, a patch, liquid or spray.

In certain circumstances, a spray can be formed in an enclosure over a treatment area.

In certain circumstances, the skin permeability enhancer can be a solvent and solubilizer.

In certain circumstances, the skin permeability enhancer can be an ether, such as a monoethyl ether.

In certain circumstances, the skin permeability enhancer can be diethylene glycol monoethyl ether.

In certain circumstances, the topical pharmaceutical composition can include an adrenergic receptor interacter. For example, the adrenergic receptor interacter can include eugenol or eugenol acetate, a cinnamic acid, cinnamic acid ester, cinnamic aldehyde, hydrocinnamic acid, chavicol, or safrole.

In certain circumstances, the adrenergic receptor interacter can be a phytoextract. For example, the phytoextract can include an essential oil extract of a clove plant, an essential oil extract of a leaf of a clove plant, an essential oil extract of a flower bud of a clove plant, or an essential oil extract of a stem of a clove plant.

In certain circumstances, the composition can include a mixed ester. For example, the mixed ester can be cellulose or a modified cellulose. The mixed ester can be synthetic or biosynthetic.

In certain circumstances, the pharmaceutical composition can include a cellulosic polymer is selected from the group of: hydroxypropylmethyl cellulose, hydroxyethyl cellulose, hydroxyethylmethyl cellulose, hydroxypropyl cellulose, methylcellulose and carboxymethyl cellulose.

In certain circumstances, the pharmaceutical composition can include a stabilizer.

In certain circumstances, the prodrug can be an ester of epinephrine.

In certain circumstances, the medical condition can include alopecia, contact hypersensitivity, aging skin, pemphigus, psoriasis, pruritis, atopic dermatitis, wounds, melanoma, vitiligo, alopecia, acne, alopecia areata, Raynaud's phenomenon, epidermolysis bullosa, rosacea, scleroderma, hidradenitis suppurativa (acne inversa), ichthyosis, pachyonychia congenital, or urticaria.

In certain circumstances, the prodrug can be an ester of epinephrine, such as dipifevrin.

In certain circumstances, epinephrine or its prodrug can impact mast cells or histamines.

In certain circumstances, epinephrine or its prodrug can target a disease in which natural killer (NK) cells are activated.

In certain circumstances, epinephrine or its prodrug target can act as immunosuppressants or melanogenic agents.

In certain circumstances, epinephrine or its prodrug can inhibit cytokine production.

In certain circumstances, epinephrine or its prodrug can elevate TNF-α.

In certain circumstances, epinephrine or its prodrug can elevate IFNΥ.

Other aspects, embodiments, and features will be apparent from the following description, the drawings, and the claims.

### BRIEF DESCRIPTION OF THE FIGURES

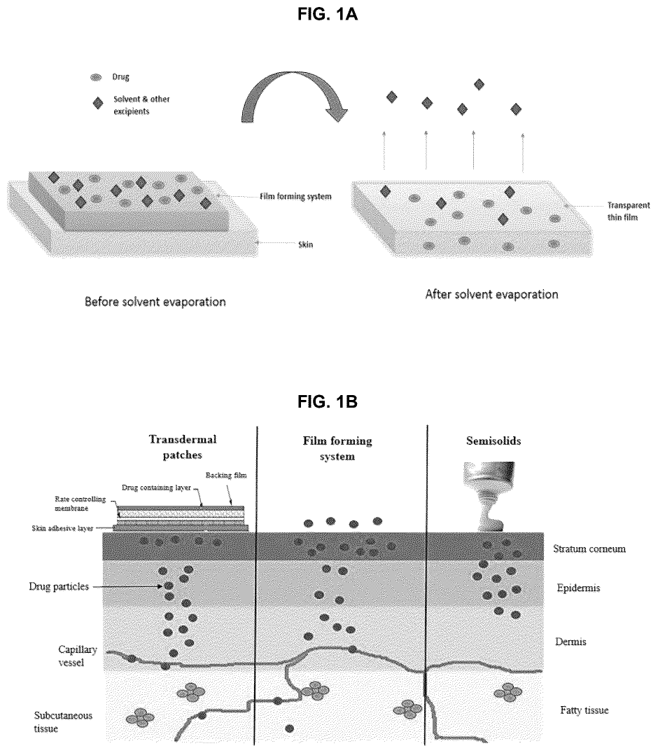

Referring to FIG. 1A, this schematic shows an exemplary method of forming a topical. pharmaceutical composition.

Referring to FIG. 1B, this schematic shows an exemplary topical pharmaceutical composition which can be a transdermal patch, a film forming system, or a semisolid.

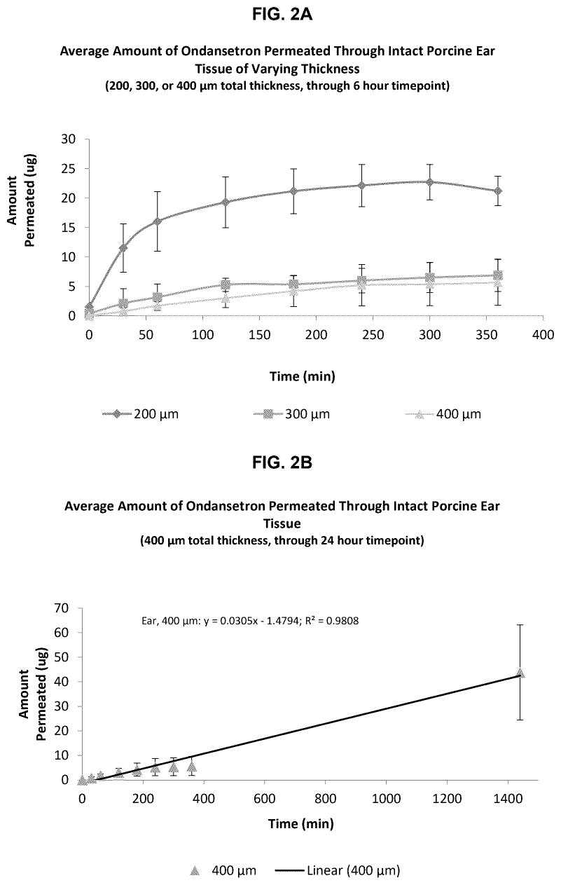

Referring to FIG. 2A, this graph shows average amount of active material permeated vs. time.

Referring to FIG. 2B, this graph shows average flux vs. time.

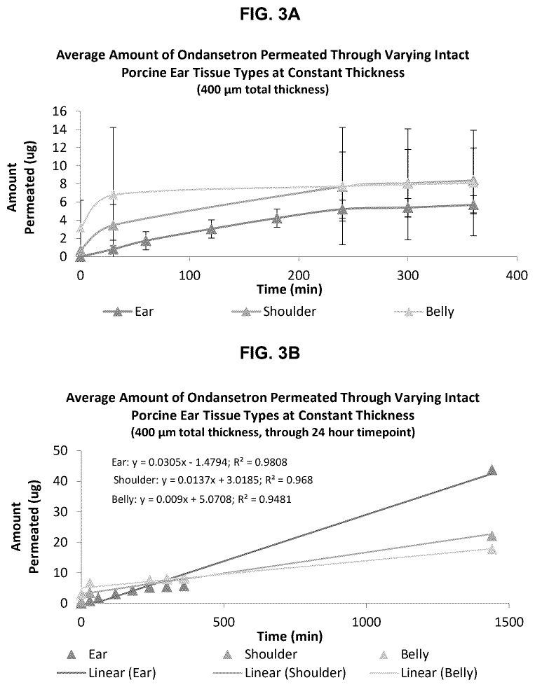

Referring to FIG. 3A, this graph shows ex-vivo permeation a function of time.

Referring to FIG. 3B, this graph shows ex-vivo permeation a function of time.

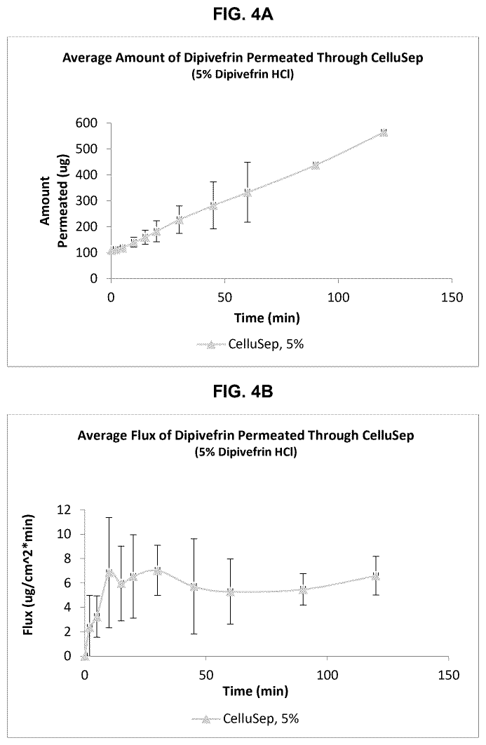

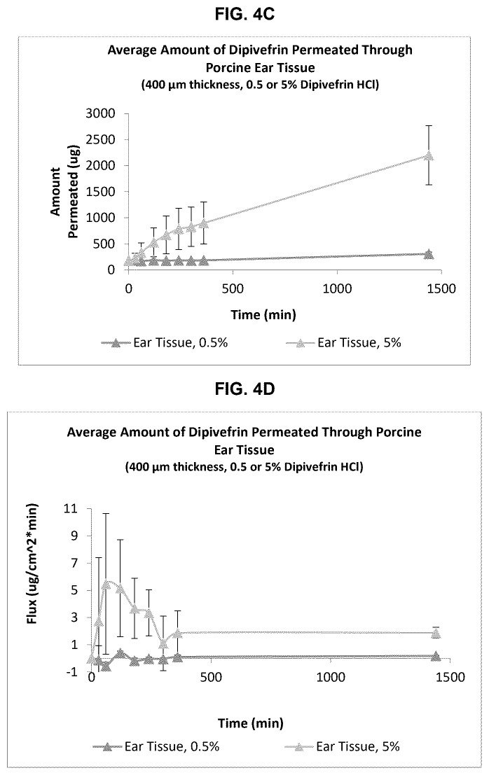

Referring to FIG. 4A, this graph shows permeation of dipivefrin as a function of time.

Referring to FIG. 4B, this graph shows flux of dipivefrin as a function of time.

Referring to FIG. 4C, this graph shows permeation of dipivefrin as a function of time.

Referring to FIG. 4D, this graph shows flux of dipivefrin as a function of time.

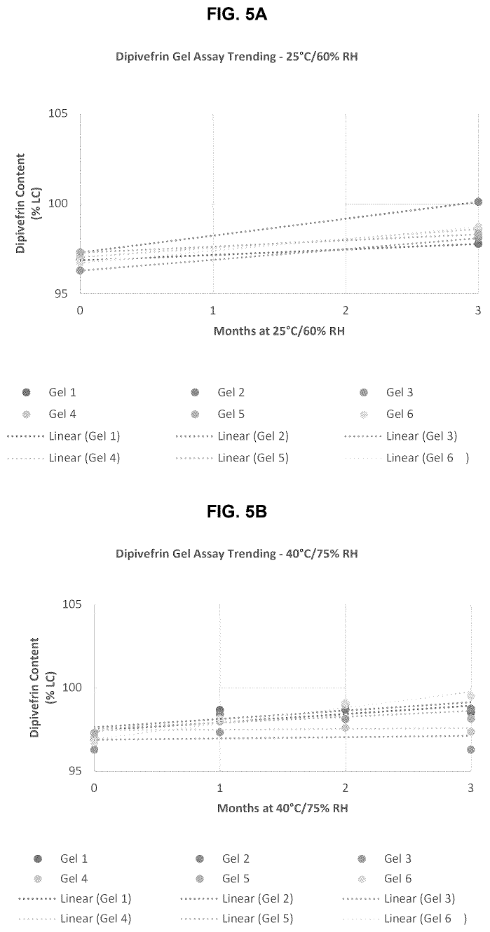

Referring to FIG. 5A, this graph shows stability data at 25° C. for a topical formulation of dipivefrin.

Referring to FIG. 5B, this graph shows stability data at 40° C. for a topical formulation of dipivefrin.

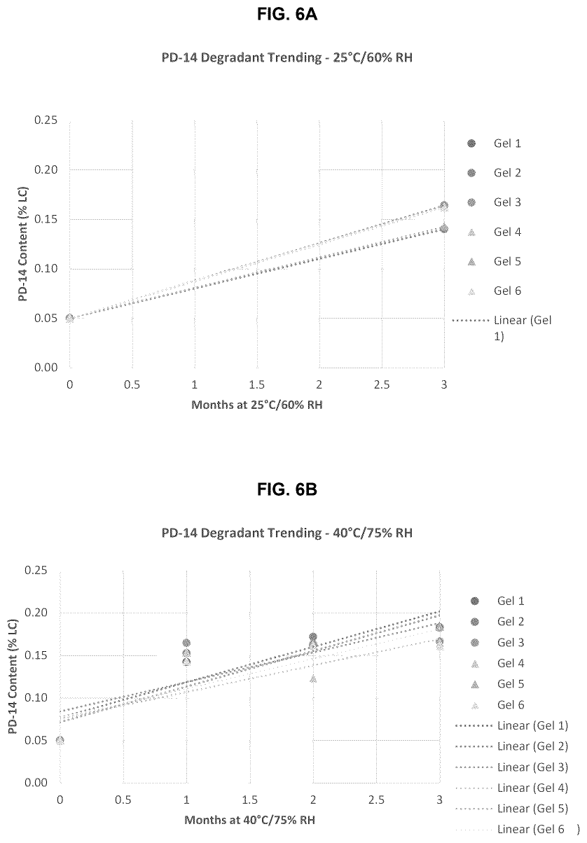

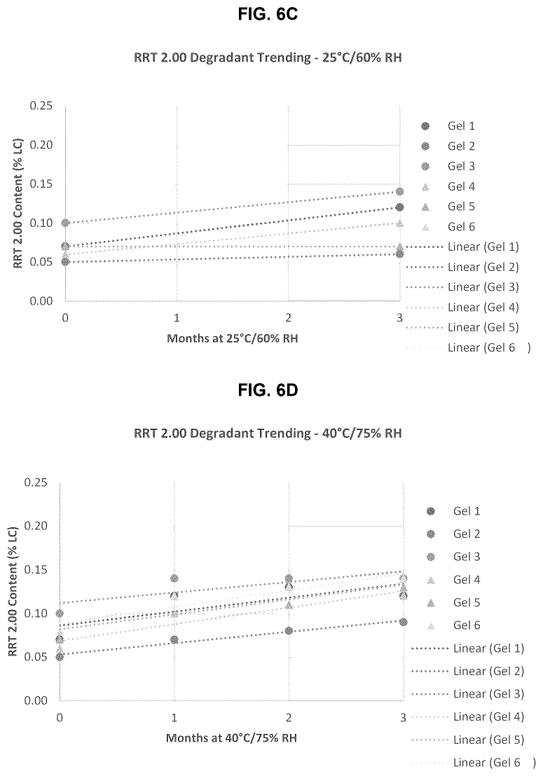

Referring to FIG. 6A, this graph shows degradant trending for a topical formulation over time.

Referring to FIG. 6B, this graph shows degradant trending for a topical formulation over time.

Referring to FIG. 6C, this graph shows degradant trending for a topical formulation over time.

Referring to FIG. 6D, this graph shows degradant trending for a topical formulation over time.

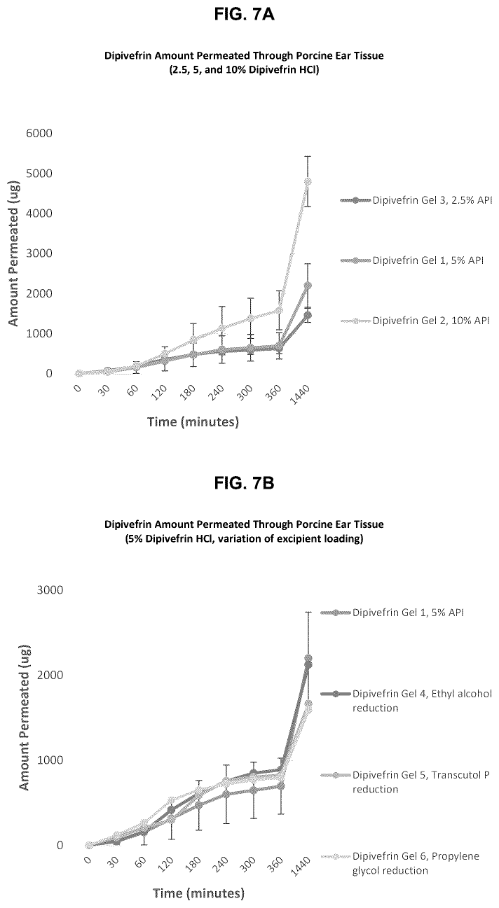

Referring to FIG. 7A, this graph shows amount of epinephrine prodrug for a topical formulation permeated over time.

Referring to FIG. 7B, this graph shows amount of epinephrine prodrug for a topical formulation permeated over time.

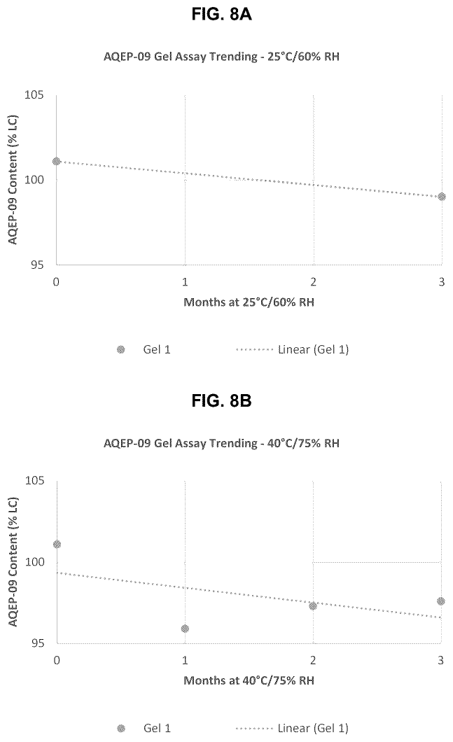

Referring to FIG. 8A, this graph shows amount of epinephrine prodrug remaining over time.

Referring to FIG. 8B, this graph shows amount of epinephrine prodrug remaining over time.

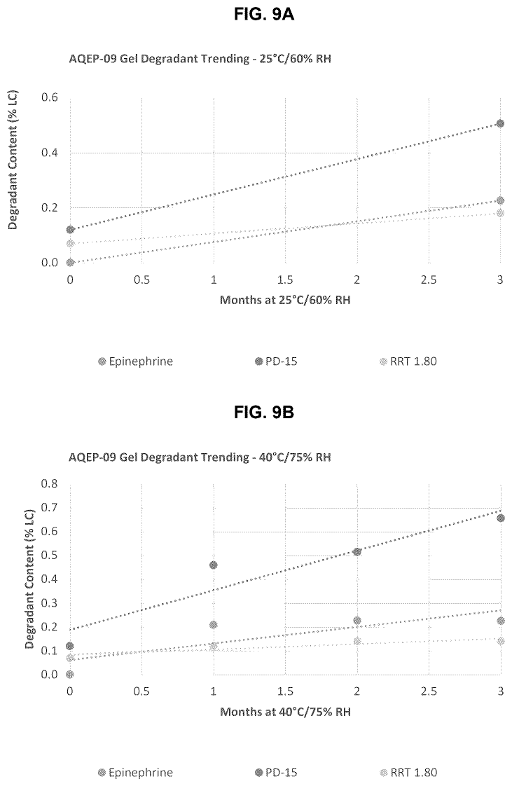

Referring to FIG. 9A, this graph shows degradant content over time.

Referring to FIG. 9B, this graph shows degradant content over time.

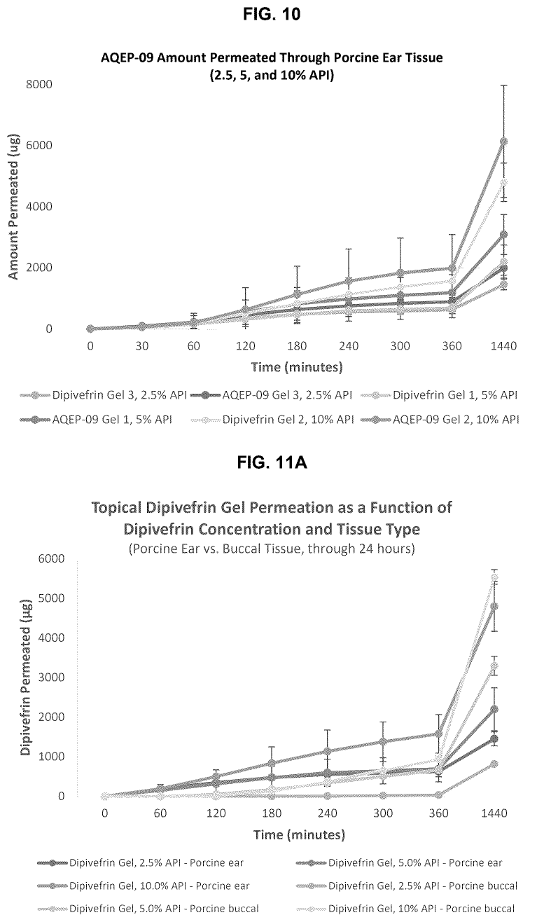

Referring to FIG. 10, this graph shows a permeation study for a topical composition of epinephrine prodrug over time.

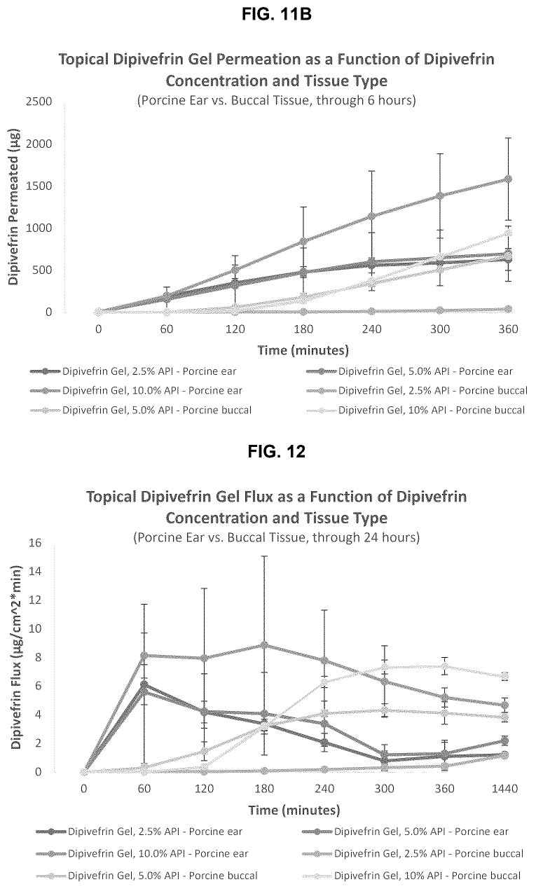

Referring to FIG. 11A, this graph shows a permeation study for a topical composition of epinephrine prodrug as a function of tissue type over time.

Referring to FIG. 11B, this graph shows a permeation study for a topical composition of epinephrine prodrug as a function of tissue type over time.

Referring to FIG. 12, this graph shows a flux study for a topical composition of epinephrine prodrug as a function of tissue type over time.

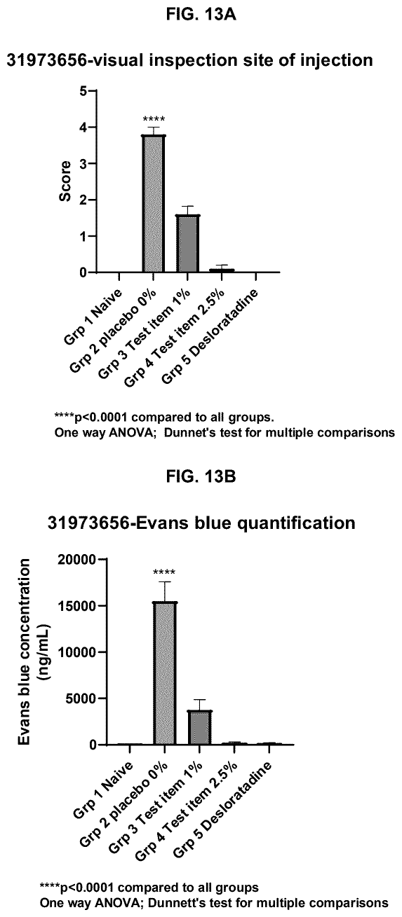

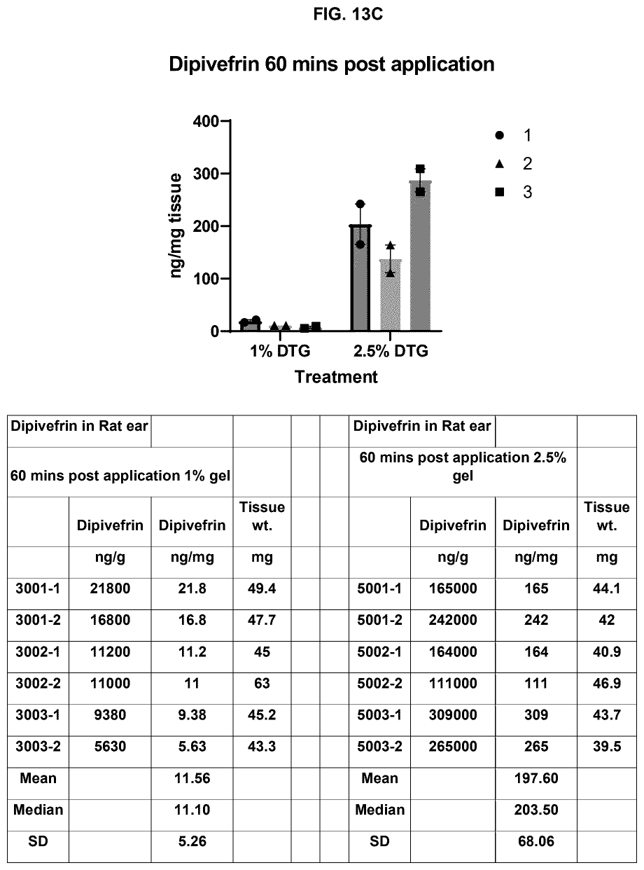

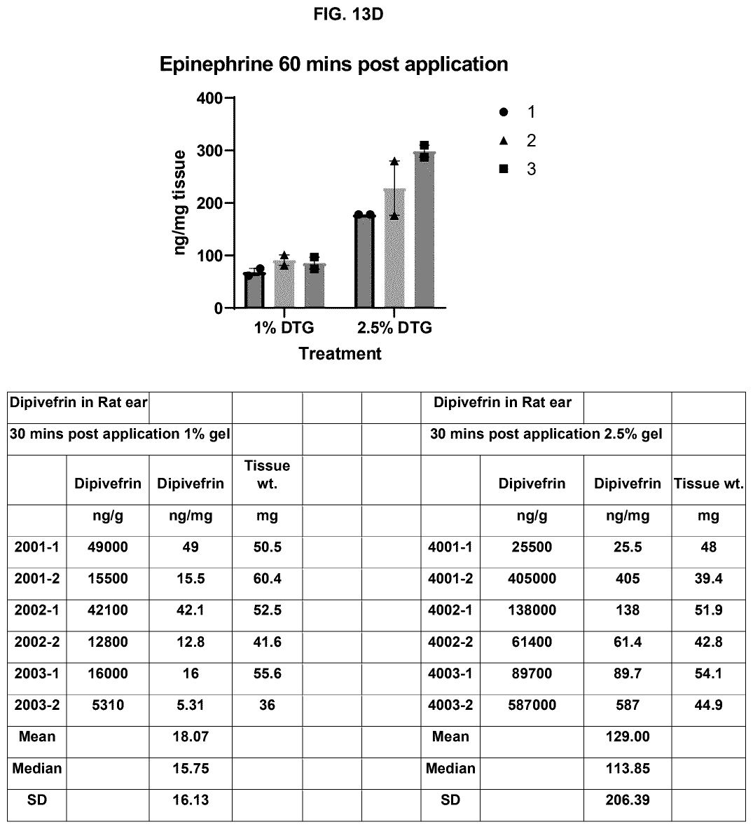

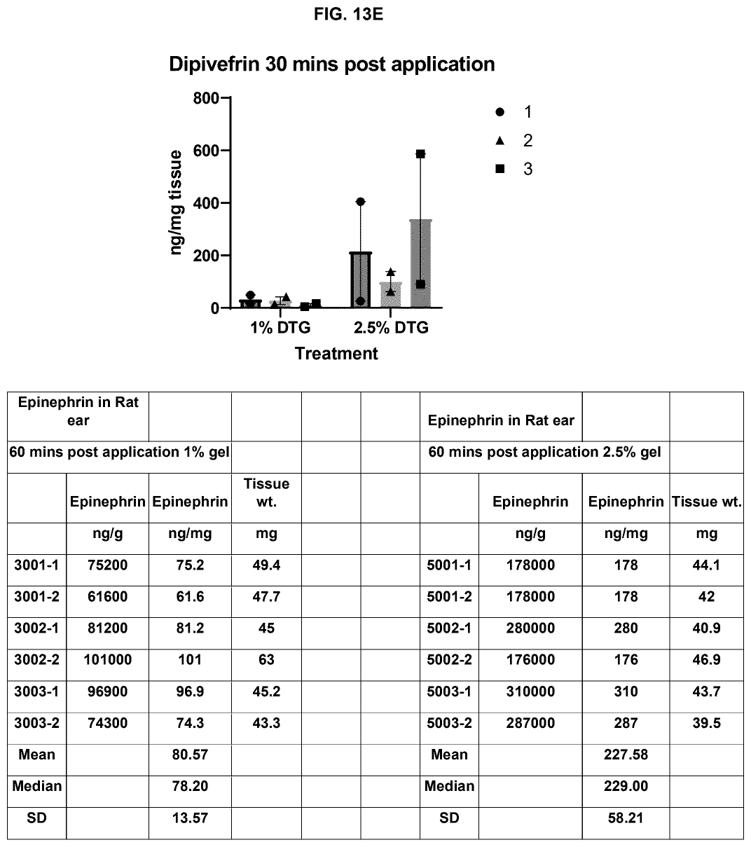

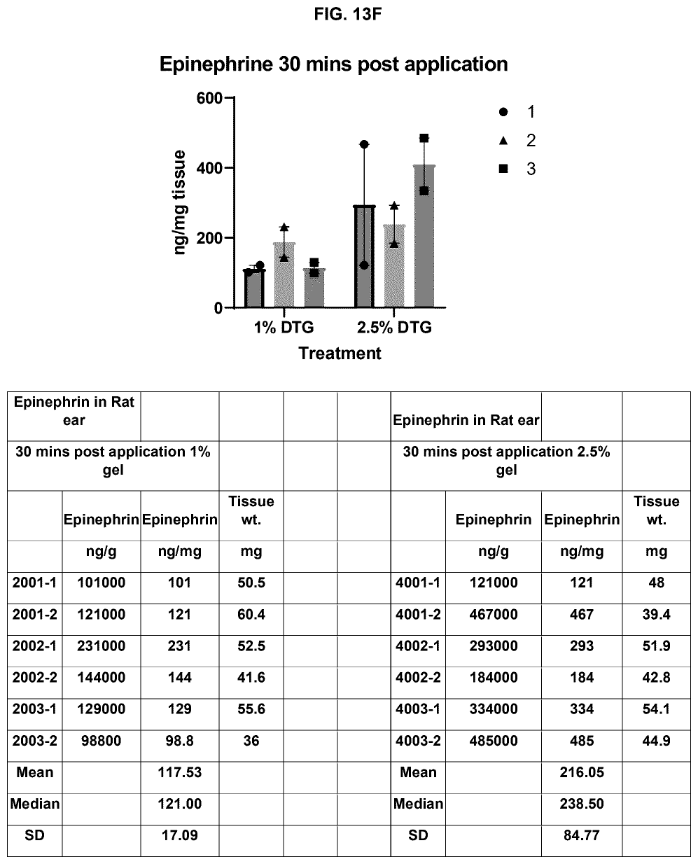

Referring to FIG. 13A, this graph depicts the test results of a pharmacokinetic study of the claimed composition topically applied on rat ears.

Referring to FIG. 13B, this graph depicts the Evans blue quantification of FIG. 13A.

Referring to FIG. 13C, this graph depicts dipivefrin concentration 60 minutes post topical application.

Referring to FIG. 13D, this graph depicts epinephrine concentration 60 minutes post topical application.

Referring to FIG. 13E, this graph depicts dipivefrin concentration 30 minutes post topical application.

Referring to FIG. 13F, this graph depicts epinephrine concentration 30 minutes post topical application.

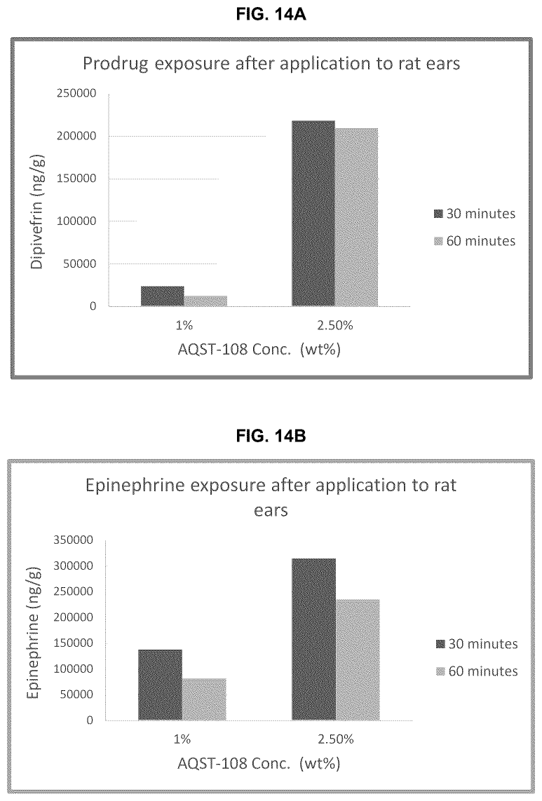

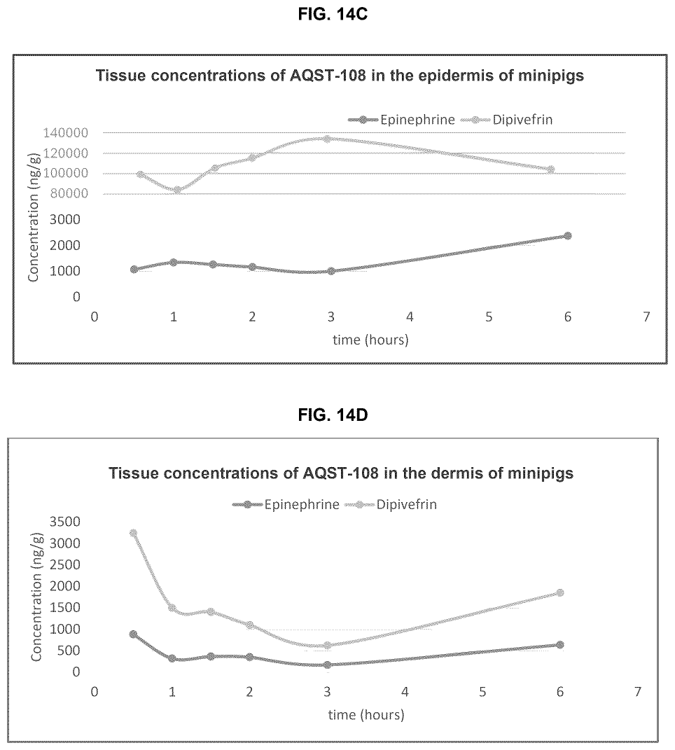

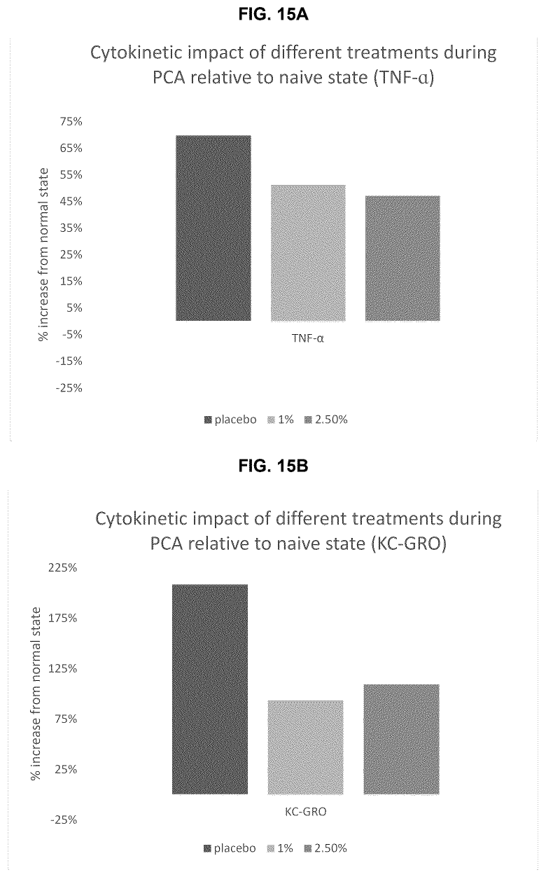

Referring to FIG. 14A, this graph depicts dipivefrin concentration in ng/g after application to rat ears comparing 30 minutes to 60 minutes.

Referring to FIG. 14B, this graph depicts epinephrine concentration in ng/g after application to rat ears comparing 30 minutes to 60 minutes.

Referring to FIG. 14C, this graph depicts tissue concentrations of dipivefrin in the epidermis of minipigs.

Referring to FIG. 14D, this graph depicts the tissue concentrations of dipivefrin in the dermis of minipigs.

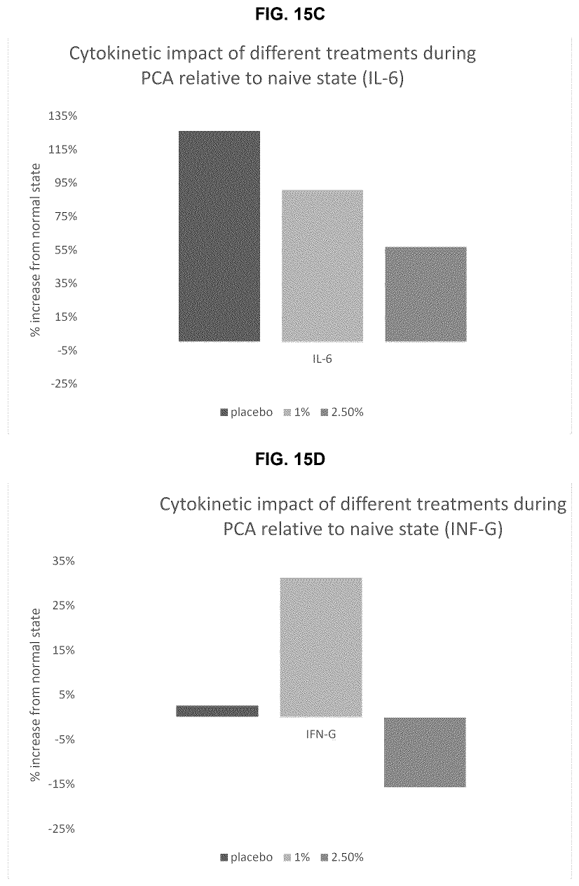

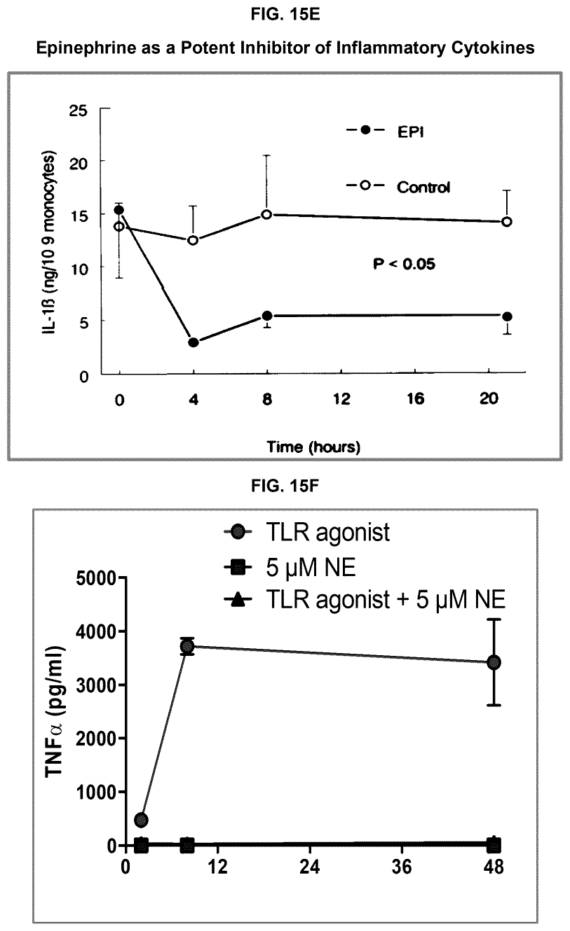

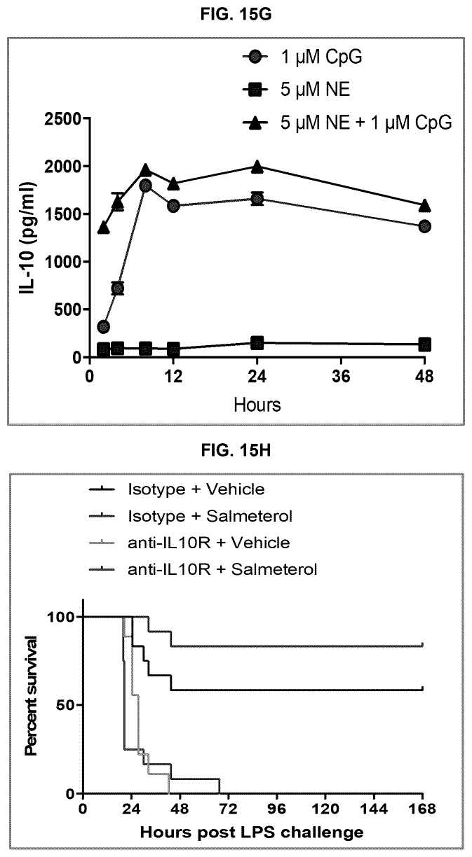

Referring to FIG. 15A, this graph depicts cytokinetic impact of the topical application during PCA relative to naïve state for TNF-α.

Referring to FIG. 15B, this graph depicts cytokinetic impact of the topical application during PCA relative to naïve state for KC-GRO.

Referring to FIG. 15C, this graph depicts cytokinetic impact of the topical application during PCA relative to naïve state for IL-6.

Referring to FIG. 15D, this graph depicts cytokinetic impact of the topical application during PCA relative to naïve state for INF-γ.

Referring to FIG. 15E, this shows epinephrine as an inhibitor of inflammatory cytokines.

Referring to FIG. 15F, this shows TNF-α suppression over time.

Referring to FIG. 15G, this shows IL-10 secretion over time.

Referring to FIG. 15H, this shows percent survival as a function of time (hours post LPS challenge).

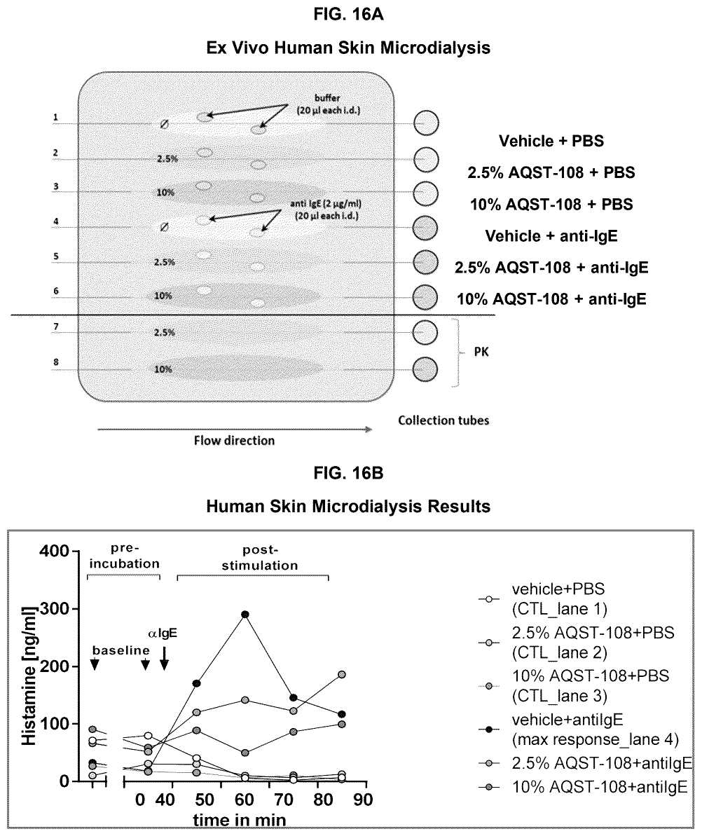

Referring to FIG. 16A, this graph shows results of a human skin microdialysis of interstitial fluid across multiple treatment groups.

Referring to FIG. 16B, this graph shows human skin microdialysis results by depicting histamine levels (ng/ml) over time.

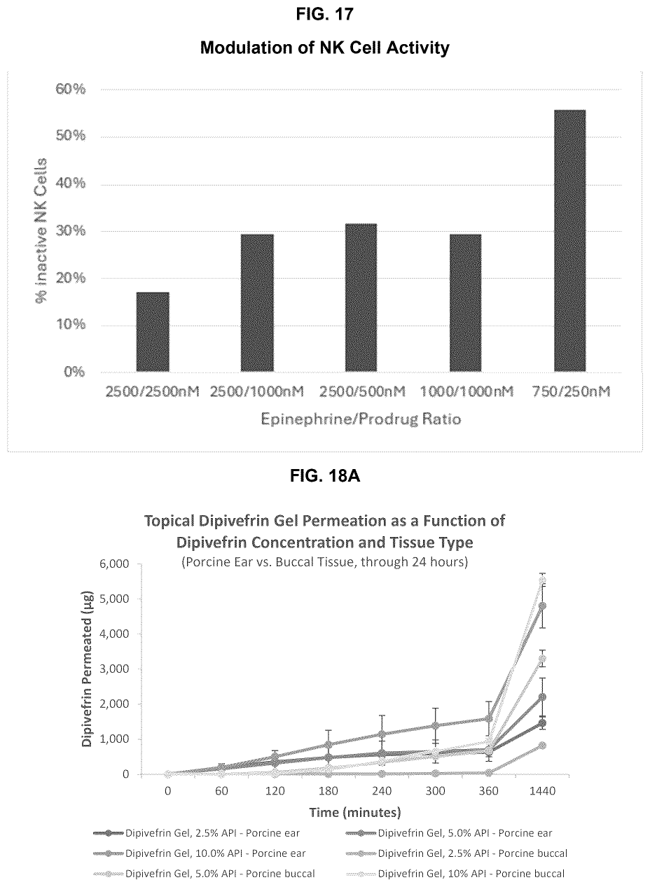

Referring to FIG. 17, this graph shows modulation of natural killer (NK) cell activity after treatment with the topical dipivefrin composition.

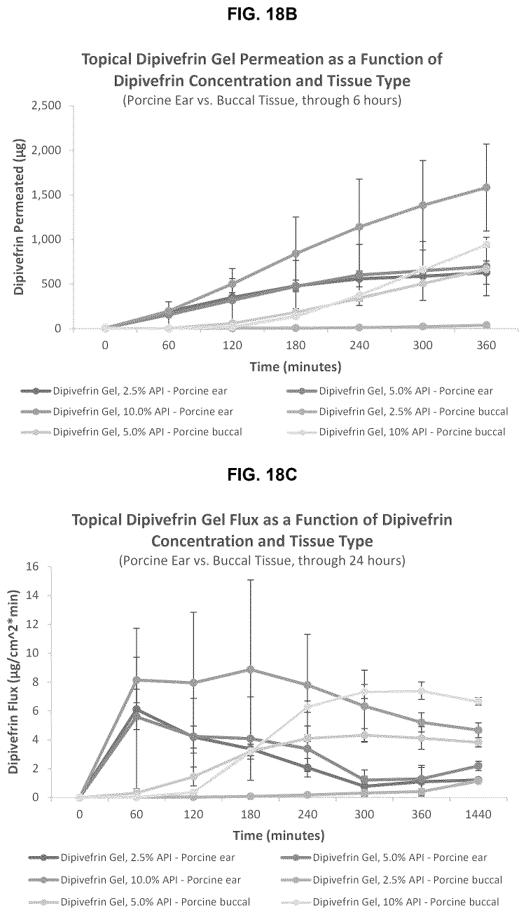

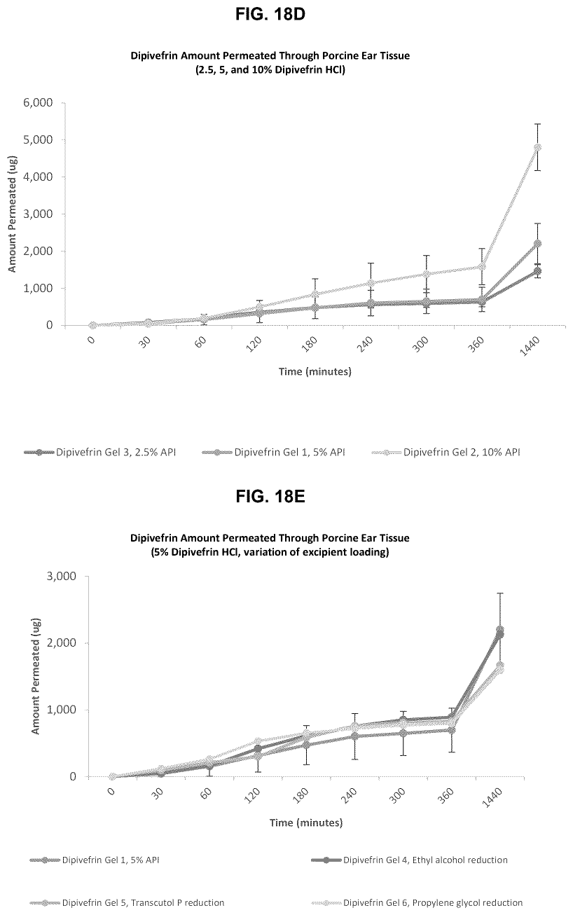

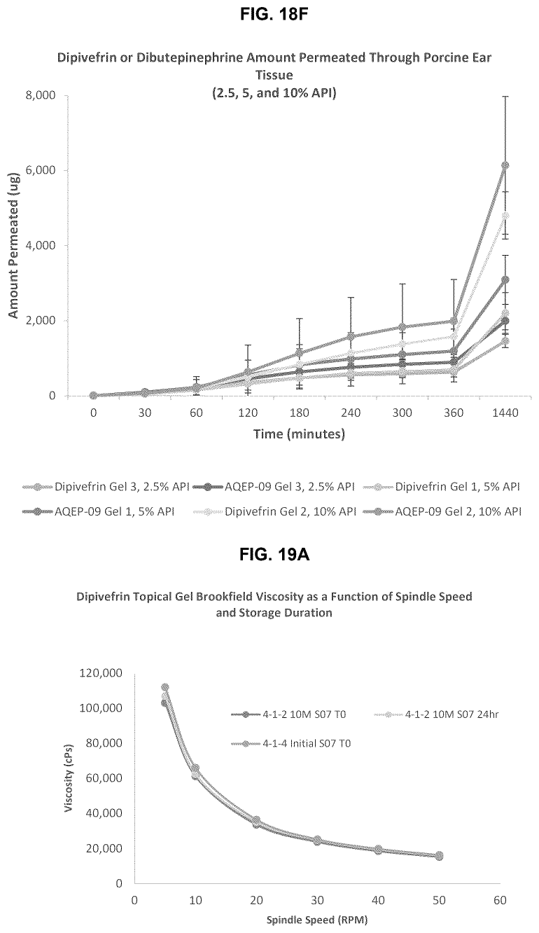

Referring to FIG. 18A, this shows the topical dipivefrin gel permeation as a function of concentration and tissue type (through 24 hours).

Referring to FIG. 18B, this shows the topical dipivefrin gel dipivefrin permeation as a function of concentration and tissue type (through 6 hours).

Referring to FIG. 18C, this shows the topical dipivefrin gel dipivefrin flux as a function of concentration and tissue type (through 24 hours).

Referring to FIG. 18D, this shows dipivefrin amount permeated through porcine ear tissue.

Referring to FIG. 18E, this shows dipivefrin amount permeated through porcine ear tissue (variation of excipient loading).

Referring to FIG. 18F, this shows dipivefrin or dibutepinephrine amount permeated through porcine ear tissue.

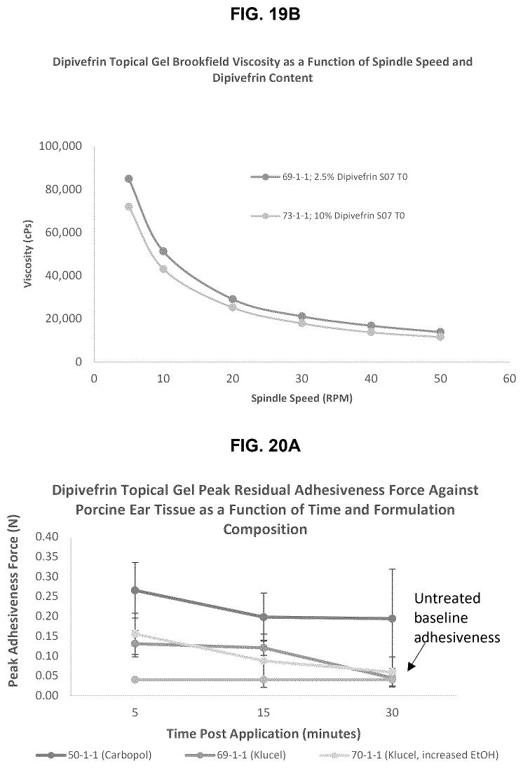

Referring to FIG. 19A, this shows dipivefrin topical gel viscosity as a function of spindle speed and storage duration.

Referring to FIG. 19B, this shows dipivefrin topical gel viscosity as a function of spindle speed and dipivefrin content.

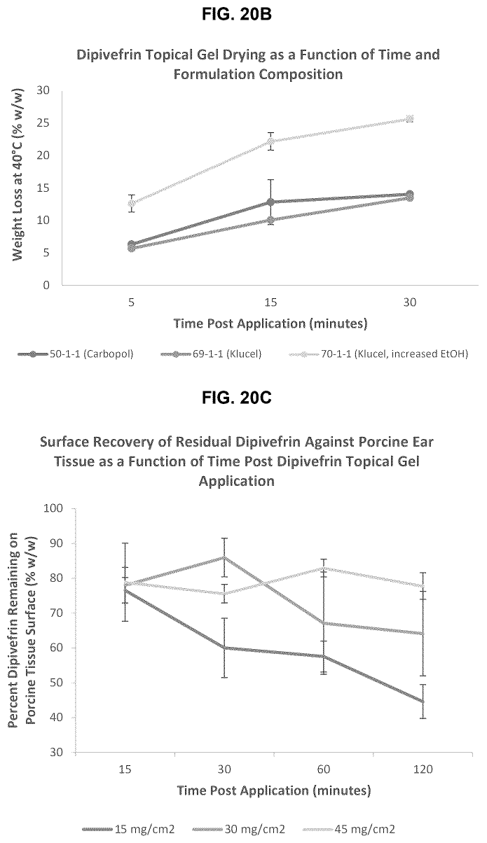

Referring to FIG. 20A, this shows dipivefrin topical gel peak residual adhesiveness force as a function of time and formulation composition.

Referring to FIG. 20B, this shows dipivefrin topical gel drying as a function of time and formulation composition.

Referring to FIG. 20C, this shows surface recovery of residual dipivefrin against porcine ear tissue as a function of time post application.

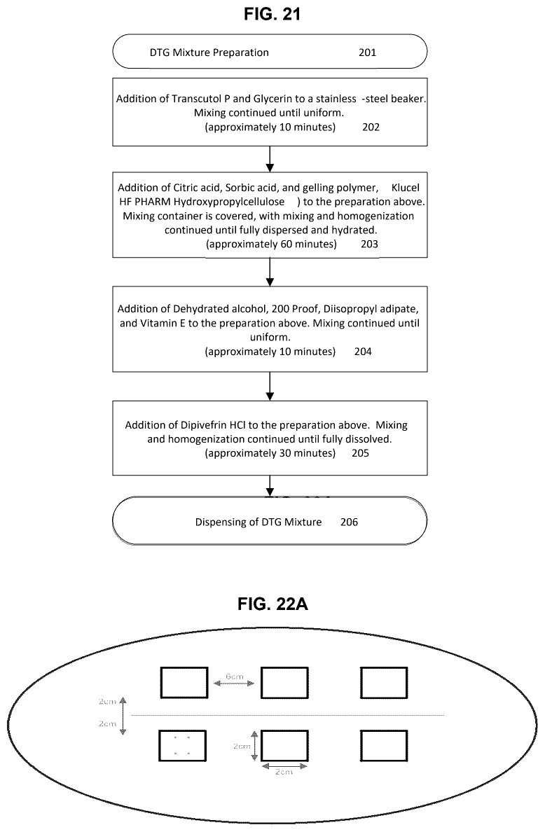

Referring to FIG. 21, this shows a method of making a topical gel formulation.

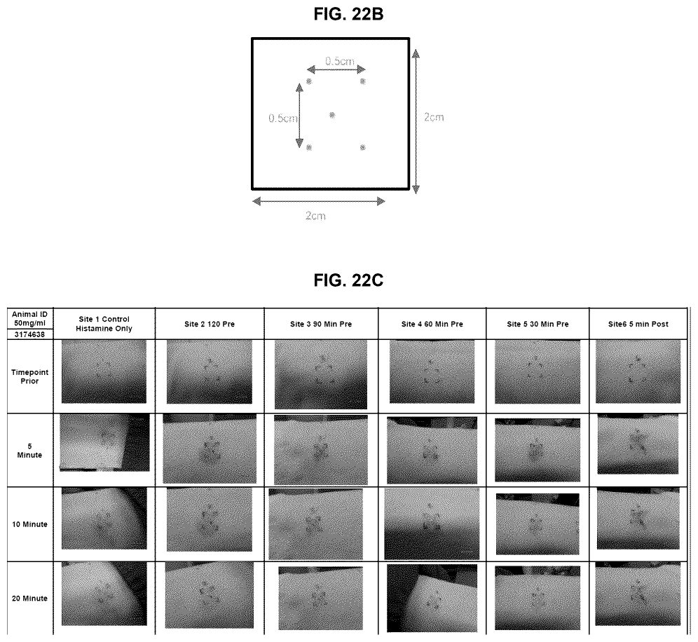

Referring to FIG. 22A, this shows the treatment sites of a urticaria reaction study

Referring to FIG. 22B, this shows the histamine injection site of a urticaria reaction study.

Referring to FIG. 22C, this shows the sites treated topically for both the low and high histamine groups of urticaria reaction study.

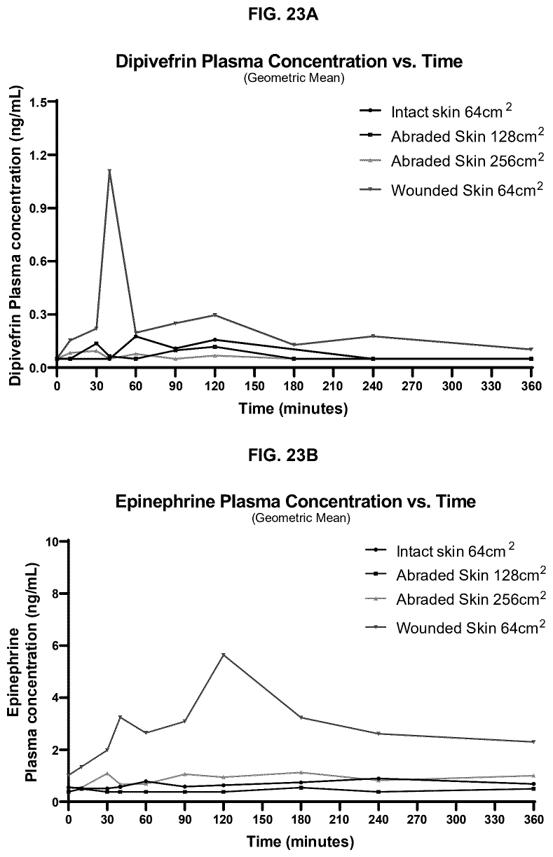

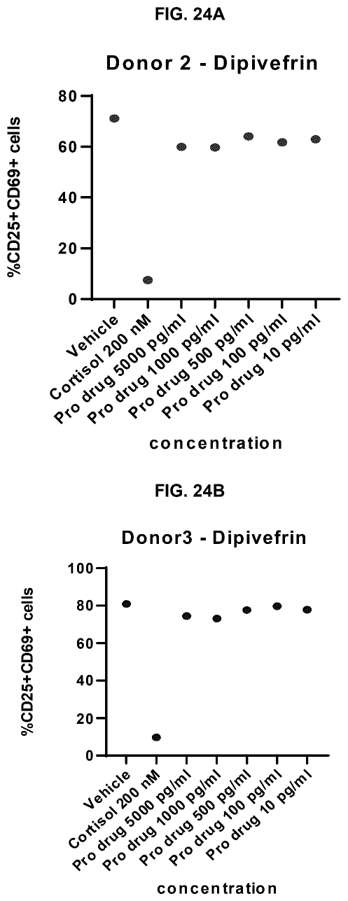

Referring to FIG. 23A, this shows dipivefrin concentration as a function of time in a skin abrasion study.

Referring to FIG. 23B, this shows epinephrine concentration as a function of time in a skin abrasion study.

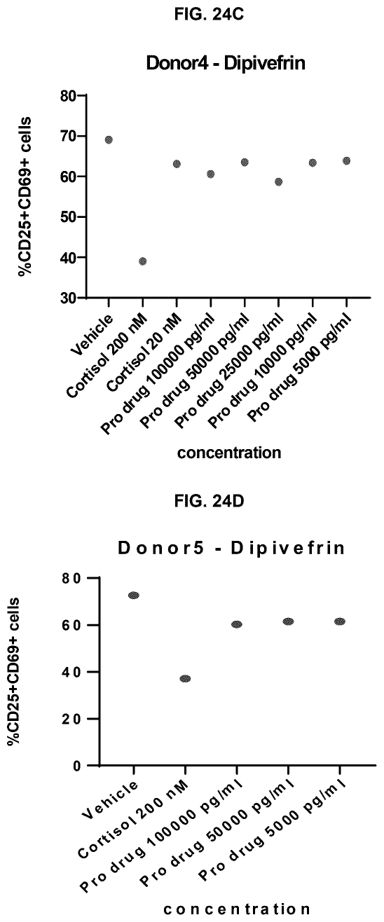

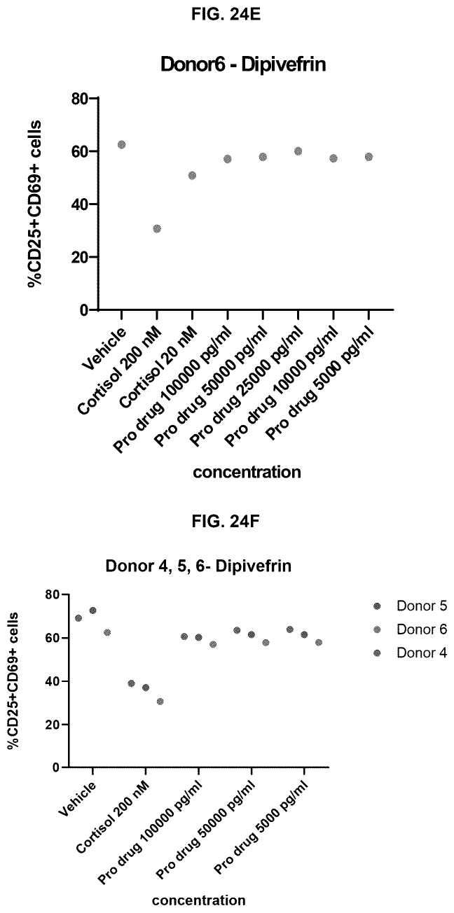

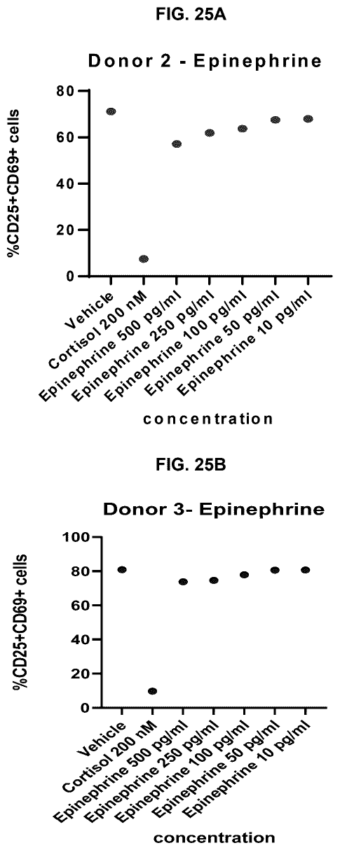

Referring to FIG. 24A, this shows the effect of dipivefrin on NK cell activation.

Referring to FIG. 24B, this shows the effect of dipivefrin on NK cell activation.

Referring to FIG. 24C, this shows the effect of dipivefrin on NK cell activation.

Referring to FIG. 24D, this shows the effect of dipivefrin on NK cell activation.

Referring to FIG. 24E, this shows the effect of dipivefrin on NK cell activation.

Referring to FIG. 24F, this shows the effect of dipivefrin on NK cell activation.

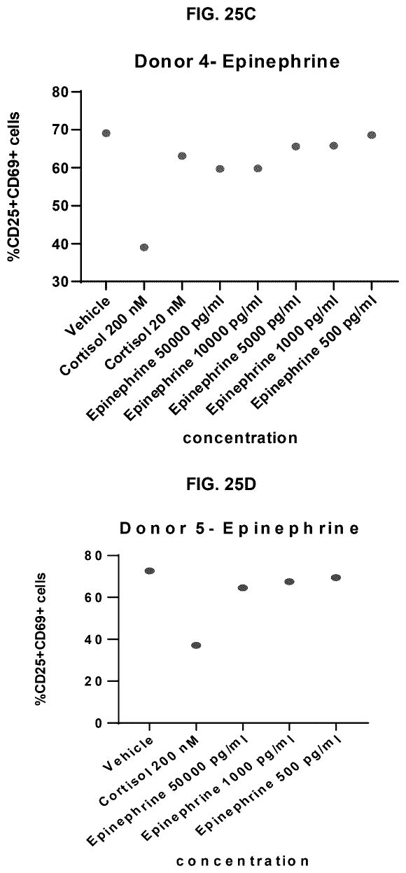

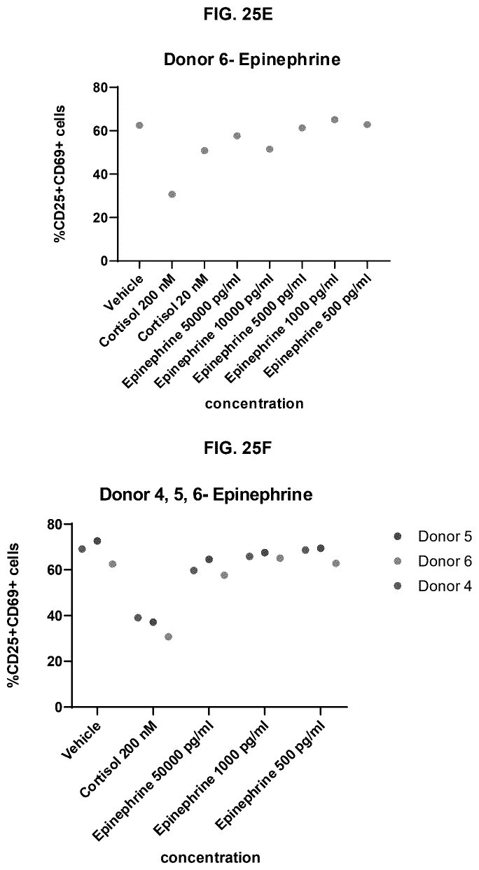

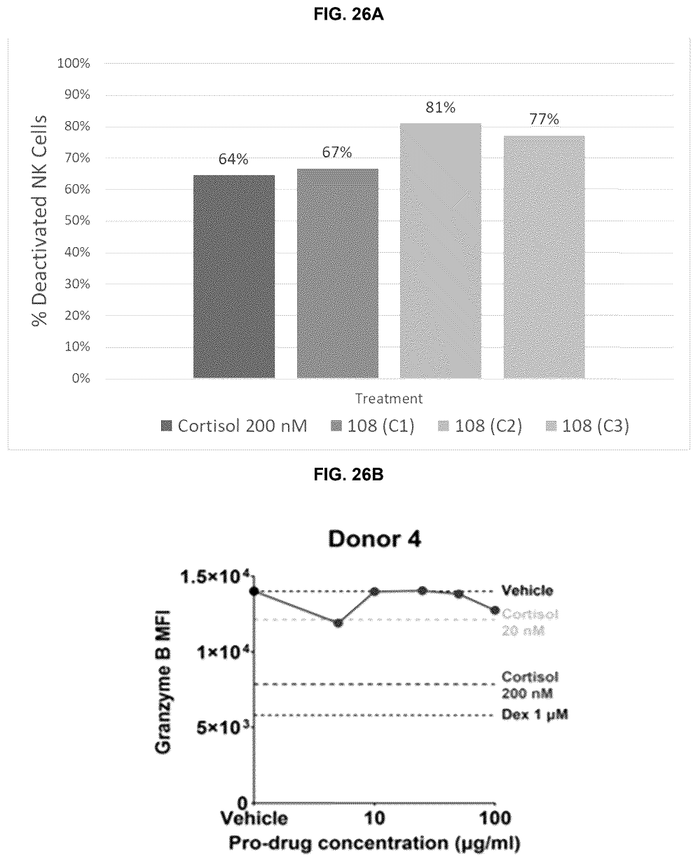

Referring to FIG. 25A, this shows the effect of epinephrine on NK cell activation.

Referring to FIG. 25B, this shows the effect of epinephrine on NK cell activation.

Referring to FIG. 25C, this shows the effect of epinephrine on NK cell activation.

Referring to FIG. 25D, this shows the effect of epinephrine on NK cell activation.

Referring to FIG. 25E, this shows the effect of epinephrine on NK cell activation.

Referring to FIG. 25F, this shows the effect of epinephrine on NK cell activation.

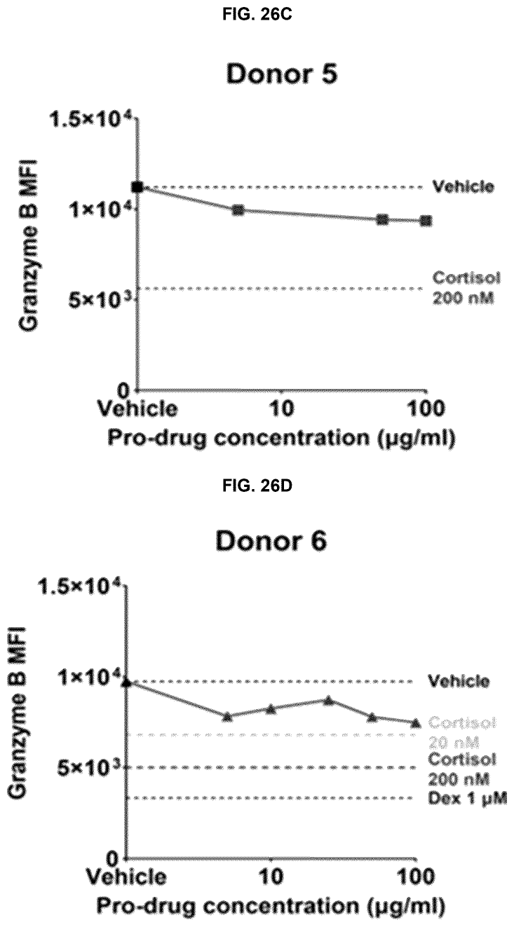

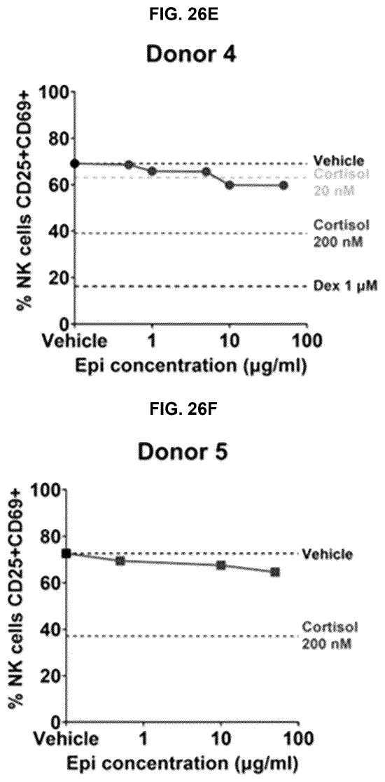

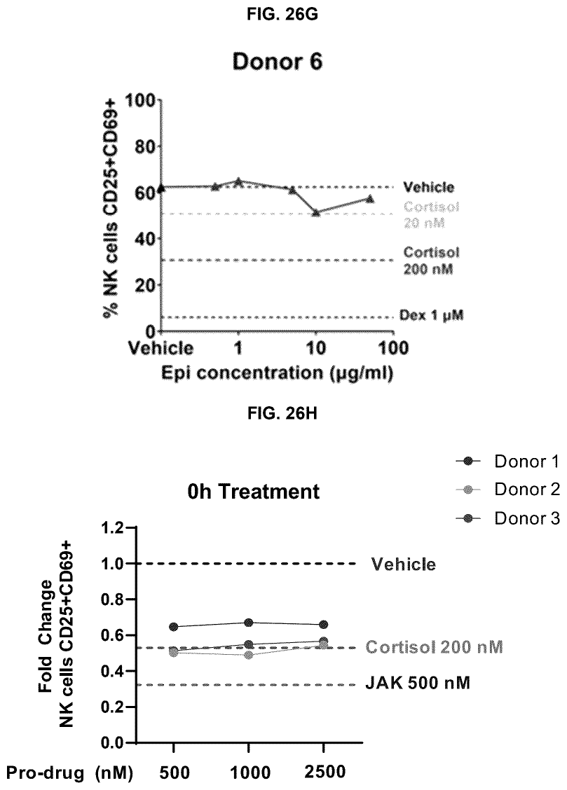

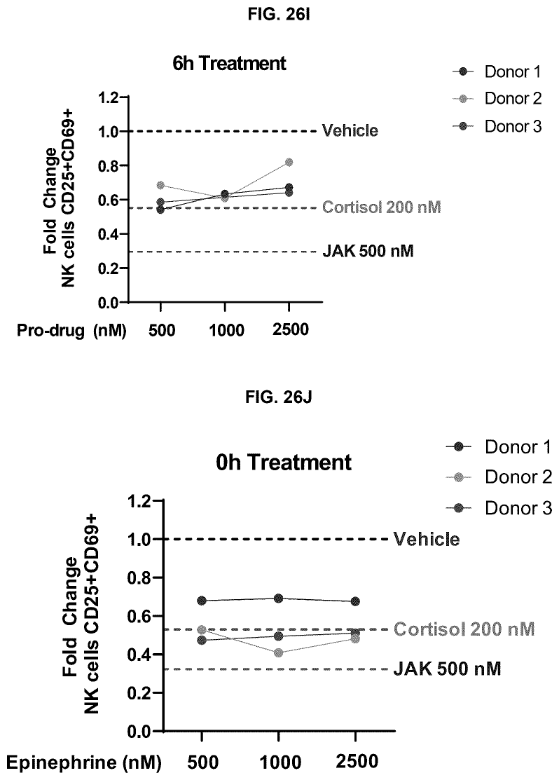

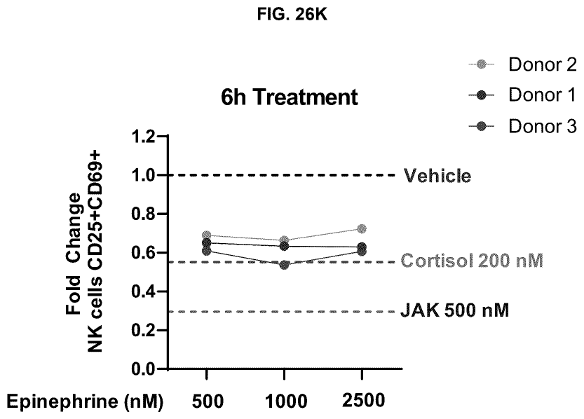

Referring to FIG. 26A, this shows the effect of epinephrine and dipivefrin combined for effective suppression of activation of NK cells.

Referring to FIG. 26B, this shows the effect of dipivefrin and epinephrine on Granzyme B MFI.

Referring to FIG. 26C, this shows the effect of dipivefrin and epinephrine on Granzyme B MFI.

Referring to FIG. 26D, this shows the effect of dipivefrin and epinephrine on Granzyme B MFI.

Referring to FIG. 26E, this shows the effect of dipivefrin and epinephrine on Granzyme B MFI.

Referring to FIG. 26F, this shows the effect of dipivefrin and epinephrine on Granzyme B MFI.

Referring to FIG. 26G, this shows the effect of dipivefrin and epinephrine on Granzyme B MFI.

Referring to FIG. 26H, this shows the effect of epinephrine and control compounds on proportion of CD25+CD69+NK cells when administered at 0 (Fold change).

Referring to FIG. 26I, this shows the effect of epinephrine and control compounds on proportion of CD25+CD69+NK cells when administered at 6 hours (Fold change).

Referring to FIG. 26J, this shows the effect of epinephrine and control compounds on proportion of CD25+CD69+NK cells when administered at 24 hours (Fold change).

Referring to FIG. 26K, this shows the effect of epinephrine and control compounds on proportion of CD25+CD69+NK cells when administered at 30 hours (Fold change)

### DETAILED DESCRIPTION

A topical composition for delivering epinephrine can include a prodrug of epinephrine. The prodrug of epinephrine can improve topical, transdermal or dermal absorption of the pharmaceutical active due to prodrug structure. In certain circumstances, the prodrug of epinephrine can improve residence time of the active due to prodrug conversion kinetics. Epinephrine mechanism of action can change based on context and duration of targeted use. Topical treatment of skin tissue with a topical composition described herein can address conditions including alopecia, contact hypersensitivity, aging skin, pemphigus, psoriasis, pruritis, atopic dermatitis, wounds, melanoma, vitiligo, acne, or urticaria and other skin disorders. The treatment or mechanism of action can have direct, indirect or a combination of the direct and indirect systemic affect on a particular disease state.

For example, in acute cases, epinephrine can impact hemostasis, which can lead to vasoconstriction. In this circumstance, the topical treatment can impact the skin tissue at or near the surface, facilitating treatment of wounds or venous ulcers. In another example, topical treatment can impact skin tissue at mast cells, serving as a mast cell stabilizer, which can lead to histamine reversal. This approach can facilitate treatment of aging skin or contact hypersensitivity. For example, mast cells are multifunctional regulators, and epinephrine can modulate the activation of mast cells. The increase in mast cells in aged skin is localized to the papillary dermis where these cells are in closer proximity to macrophages but have reduced interaction with the microvasculature and other immune populations. In aged skin, mast cells also exhibit lower amounts of degranulation and form closer interactions with macrophages and vasoactive intestinal peptide-positive nerve fibers while lessening their association with the dermal vasculature. Pilkington, S. M., Aged Human Skin Accumulates Mast Cells with Altered Functionality that Localize to Macrophages and Vasoactive Intestinal Peptide-Positive Nerve Fibres, British J. of Dermatology (2019) 180, pp. 849-858.

In more chronic situations, the topical treatment can serve as a hemodynamic stabilizer, for example, by maintaining mean arterial pressure (MAP), which can in turn treat hypotension. In other chronic situations, the topical treatment can be an immunosuppressant, for example, a lymphocyte inhibitor. This treatment can target vitiligo, psoriasis, pemphigus or alopecia areata and other disorders.

The composition can impact the behaviour of cell lines within the skin and skin structures that express, either induced or constitutively, adrenergic receptors, inclusive of keratinocytes, dendritic cells, T cells (inclusive of Treg cells), B cells, macrophages, melanocytes, NK cells, keratinocytes, Langerhans cells, or Merkel cells.

It has been shown that α₁-AR stimulation increases IL-1B production in human monocytes responding to LPS. A1-AR modulations of TLR4 signaling may prove useful therapeutic strategy for management of human diseases with known chronic inflammatory etiologies. Grisanti, L., α₁-Adrenergic Receptors Positively Regulate Toll-Like Receptor Cytokine Production from Human Monocytes and Macrophages, The Journal of Pharmacology and Experimental Therapeutics, (2011) Vol. 338, No. 2, 648-657, which is incorporated by reference in its entirety.

Immune cells come in direct contact with the dendrites of the neurons in the sympathetic nervous system. Sympathetic nerves secrete norepinephrine in response to pathogenic organisms. While signaling through pattern recognition receptors (PRRs) promotes inflammatory cytokine secretion from antigen-presenting cells, neurons themselves express various Toll-like receptors (TLRs), enabling them to respond directly to certain pathogen-associated molecular patterns. Sharma and Farrar, Adrenergic regulation of immune cell function and inflammation, Seminars in Immunopathology (2020) 42: 709-717, which is incorporated by reference in its entirety.

The penetration of the topical composition into the skin tissue can influence the condition to be treated. For example, if penetration is limited, for example, to the epidermis, the treatment can impact healing of a wound. If penetration is deeper into the skin tissue, for example, to the dermis, the treatment can impact psoriasis, alopecia, aging skin, contact hypersensitivity, immunocompromised disorders or atopic dermatitis. Treatment of murine epidermal cell preparations with epinephrine or norepinephrine has been shown to inhibit antigen presentation in vitro. Pretreatment of epidermal cells with epinephrine or norepinephrine suppressed the ability of these cells to present adrenergic agonists. Seiffert, K., Catecholamines Inhibit the Antigen-Presenting Capability of Epidermal Langerhans Cells, J. Immunol. 2002, 168:6128-6235, which is incorporated by reference in its entirety.

The composition can be tailored through formulation of components, including mixtures of prodrugs of epinephrine, such as esters of epinephrine described below, to have a particular half life and penetration profile to impact key structures in skin tissue. For example, the composition can be formulated to target mast cells. Degranulating mast cells release a host of inflammatory agents, including histamine and various cytokines that impact multiple cutaneous diseases such as mast cell disorders, or dermatitis. The topical compositions described herein can impact these effects through stabilization of mast cells or impacting involved cytokines by epinephrine. Epinephrine delivered using the topical composition can inhibit NK cell cytotoxicity and cytokine production. NK cells are a third group of lymphocyte in addition to T and B cells. In humans they are defined as CD3− CD56+ lymphocytes. The major functions of NK cells are cytotoxicity and cytokine production. Cytotoxicity strongly increases when NK cells are stimulated by cytokines like IL-2, IL-15, IL-18 and others. NK cell functions are governed by a balance between activating messages transmitted by there ARs and inhibitory signals transmitted by their inhibitory receptors. Autologous human lymphocytes with NK cell receptors, when injected into nonlesional skin grafts from psoriatic patients on mice with severe combined immunodeficiency, caused the classic pathology of psoriasis, such as epidermal thickness, proliferation and expression of HLA-DR, intercellular adhesion molecule 1, CD1d and K-16. Von Bubnoff, Natural Killer Cells in Atopic and Autoimmune Diseases of the Skin, J. Allergy Clin. Immunol., 2010, Vol. 125, No. 1, 60-68, which is incorporated by reference in its entirety. The effectiveness of the composition can be dependent on duration of the exposure, concentration of the exposure as well as dose and influence by other cytokines and factors. For example, following administration of epinephrine, an increase of NK cells was observed starting as early as 5 minutes after injection, but contrast to NK cells, a decreased number of CD3+ and CD4_lymphocytes did not change. Oberbeck, “Catecholamines: Physiological Immunomodulators During Health and Illness,” Current Medicinal Chemistry, 2006, (13) 1979-1989, which is incorporated by reference in its entirety. Diseases in which NK cells are activated would therefore be a target for topical delivery of epinephrine. Also, because epinephrine acts as an immunosuppressant and melanogenic agent, TNFα and IFNγ can be elevated, impacting diseases like vitiligo.

Furthermore, adrenergic activation can similarly result in tolerogenesis via activation of other categories of lymphocytes such as Treg cells with co-administration at specific doses and concentrations resulting in allergy and/or autoimmune disease mitigation and at other doses acting as a vaccine adjuvant and enhancing host antigen recognition. Certain IgE-reactive autoantigens were identified as human analogs of environmental allergens. The homologous autoantigens showed structural similarity with the corresponding exogenous allergens, and IgE-cross reactivity was detected in many cases. Furthermore, certain of these cross-reactive autoantigens induced cell activation and immediate and late phase skin reactions in sensitized individuals, however mostly to a much lower degree than the cross-reactive exogenous allergens. It has also been shown that bullous pemphigoid IgE reproduces the early phase of lesion development in human skin. Valenta, R. Linking allergy to autoimmune disease, Trends in Immunology, 2008, Vol. 30, No. 3, 109-115, which is incorporated by reference in its entirety. In patients with food allergy, serum IL-6 levels were shown to discriminate between IgE-mediated and non-IgE-mediated and non-IgE mediated subgroups. A decrease in levels of IL-5 and IL-13 secreted by PBMCs has been repeatedly observed after oral or sublingual immunotherapy with food allergens, whereas the data on IL-4, IL1-, TGF-ß and IFNγ are inconsistent. Radonjic-Hoesli, Are Blood Cytokinds Reliable Biomarkers of Allergic Disease Diagnosis and Treatment Responses? J. Allergy Clin. Immunol., August 2022, 251-256, which is incorporated by reference in its entirety.

Thus, the topical compositions for topical delivery of prodrugs (individually or as a mixture) can have the advantage of a lack of systemic exposure, controllable residence time within the dermal layer, adjustable dose and dosing regimen for efficacy (per indication), and compositions with optimized stability, as well as unexpected contributions from prodrugs of epinephrine in general.

A pharmaceutical composition can be designed to deliver a pharmaceutically active component in a deliberate and tailored way. However, solubility and permeability of the pharmaceutically active component in vivo, in particular, in the mouth of a subject, can vary tremendously. A particular class of permeation enhancer can improve the uptake and bioavailability of the pharmaceutically active component in vivo. In particular, when delivered to a skin surface, the permeation enhancer can improve the permeability of the pharmaceutically active component to cells of a tissue, and, optionally, into the blood stream of the subject. The permeation enhancer can improve absorption rate and amount of the pharmaceutically active component by more than 5%, more than 10%, more than 20%, more than 30%, more than 40%, more than 50%, more than 60%, more than 70%, more than 80%, more than 90%, more than 100%, more than 150%, about 200% or more, or less than 200%, less than 150%, less than 100%, less than 90%, less than 80%, less than 70%, less than 60%, less than 50%, less than 40%, less than 30%, less than 20%, less than 10%, or less than 5%, or a combination of these ranges, depending on the other components in the composition.

A topical pharmaceutical composition can deliver a pharmaceutically active component or a prodrug of a pharmaceutically active component with enhanced permeation and solubility and improved stability. An anhydrous gel design allows for water-soluble delivery platform. The solvents for the polymer base may include an alcohol (e.g., ethyl alcohol), propylene glycol, and a skin permeability enhancer. A single-phase platform avoids concerns inherent in maintaining distinct oily/aqueous phases.

Topical permeation of a component of a composition can be altered by employing a hydrophilic-lipophilic balance (HLB). This can include selecting the size and strength of the hydrophilic and lipophilic moieties of a surfactant molecule. Tables A and B illustrate examples of permeation enhancers for topical applications. Table A is a limited sampling of permeation enhancers separated these into categories based on chemical type. Table B outlines selected physicochemical properties for permeation enhancers/solvents currently being evaluated in the topical gel platforms described herein.

TABLE A
Examples of Chemical Permeation Enhancers
Used in Topical Applications

| Chemical Permeation | |
|---|---|
| Enhancer Category | Chemical Permeation Enhancer |
| Alcohol | Ethanol |
| | Benzyl alcohol |
| | Isopropyl alcohol |
| | n-Butanol |
| | Oleyl alcohol |
| | Lauryl alcohol |
| Amide | Dimethyl formamide |
| Colloid | Colloidal silica |
| Ester | Isopropyl myristate |
| | Propylene carbonate |
| Ether | Dimethyl isosorbide |
| | Triacetin |
| Fatty acid | Oleic acid |
| Fatty esters | Dioctyl adipate |
| | Dioctyl phthalate |
| | Dioctyl sebacate |
| | Di-n-hexyl phthalate |
| Higher alkanes | Mineral oil |
| Lactams | 2-Pyrrolidone |
| | Azone (1-Dodecylazacyloheptan-2-one) |
| Non-ionic | Polyoxyethylene 20 |
| surfactant | Polyoxyethylene 40 |
| | Polyoxyethylene 80 |
| | Decyl glucoside |
| | Sorbitan monolaurate (Span ® 20) |
| Phospholipid | Dimyristoylphosphatidylcholine (DMPC) |
| | Dicetyl phosphate (DCP) |
| Polyol | Propylene Glycol |
| | Glycerol |
| | Ethylene glycol |
| | Polyethylene glycol |
| | Diethylene glycol monoethyl ether (DEGEE, |
| | Transcutol P) |
| Sulfoxides | Dimethyl sulfoxide (DMSO) |
| | Decyl methyl sulfoxide (DCMS) |
| Terpine | Limonene |
| Triglyceride | Medium-chain triglycerides |

TABLE B
Selected Physicochemical Properties for Permeation
Enhancers Used in Topical Gel Platforms

| | Chemical Permeation Enhancer | | |
|---|---|---|---|
| | Diethylene glycol | | |
| | monoethyl ether | Propylene | |
| Attribute | (DEGEE) | glycol | Ethanol |
| Molecular weight (g/mol) | 134.175 | 76.1 | 46.07 |
| Density (g/cm³ at 20° C.) | 0.989 | 1.038 | 0.789 |
| Log P (octanol:water) | −0.43 | −0.92 | −0.18 |
| Vapor pressure (Pa) | 16 | 9 | 5,950 |
| Surface tension (mN/m | 31.3 | 40.1 | 21.8 |
| at 25° C.) | | | |
| Hydrophilic lipophilic | 4 | 5-6 | 8 |
| balance (HLB) | | | |

Prodrug design is an important part of drug discovery and can offer many advantages over parent drugs such as increased solubility, enhanced stability, improved bioavailability, reduced side effects, enhanced stereochemistry, reduced steric hindrance and better selectivity. The selection and design of the prodrug can be affected by the site of drug delivery, the tissue type, enzymatic conversion, steric hindrance, and other molecular considerations and interactions.

Delivery of drugs or pharmaceuticals transdermally or transmucosally can require that the prodrug, drug, active or pharmaceutical alone or in combination permeate or otherwise cross at least one biological membrane or tissue partially or completely in an effective and efficient manner.

In general, a method of treating a medical condition in a human subject can include administering a composition including a prodrug and a permeation enhancer from a matrix and the permeation enhancer promoting permeation of the prodrug through or into skin tissue to achieve an effective plasma concentration of a pharmaceutically active form of the prodrug in the human subject in less than one hour.

In certain embodiments, the method of treating a medical condition can further including administering a pharmaceutically active ingredient with the prodrug.

In certain embodiments, the composition including a prodrug includes more than one prodrug with each prodrug being a derivative of a pharmaceutically active ingredient. In some of these embodiments, one of the prodrugs is dipivefrin.

In certain embodiments, the first prodrug is a first ester of epinephrine and the second prodrug is a second ester of epinephrine, the first ester of epinephrine and the second ester of epinephrine being different.

In certain embodiments, the prodrug is a compound of formula (I), wherein

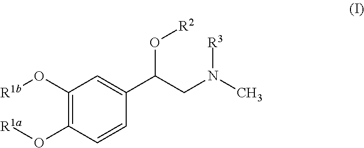

each of R^{1a}, R^{1b}, R² and R³, independently, can be H, C1-C16 acyl, alkyl aminocarbonyl, alkyloxycarbonyl, phenacyl, sulfate or phosphate, or R^{1a} and R^{1b} together, R^{1a} and R² together, R^{1a} and R³ together, R^{1b} and R² together, R^{1b} and R³ together, or R² and R³ together form a cyclic structure including a dicarbonyl, disulfate or diphosphate moiety, provided that one of R^{1a}, R^{1b}, R² and R³ is not H, or a pharmaceutically acceptable salt thereof.

In certain embodiments, R² and R³ are H and each R^{1a} and R^{1b}, independently, can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl. In some embodiments, both of R^{1a} and R^{1b} can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl. In some embodiments, one of R^{1a} and R^{1b} can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl. In some embodiments, one or both of R^{1a} and R^{1b} can be ethanoyl, n-propanoyl, or n-butanoyl.

Exemplary prodrugs are provided in the table below.

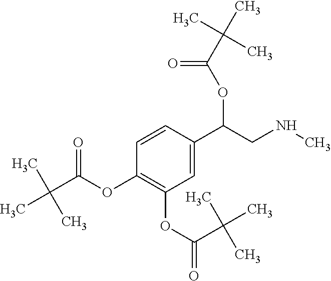 [SMILES: CNCC(OC(=O)C(C)(C)C)c1ccc(OC(=O)C(C)(C)C)c(OC(=O)C(C)(C)C)c1]

AQEP-01

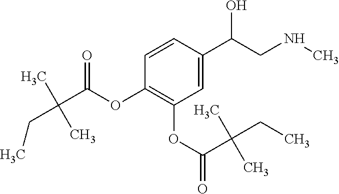 [SMILES: CCC(C)(C)C(=O)Oc1ccc(C(O)CNC)cc1OC(=O)C(C)(C)CC]

AQEP-02

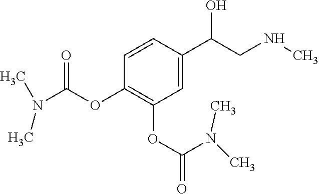

AQEP-03

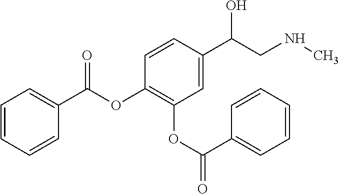 [SMILES: CNCC(O)c1ccc(OC(=O)c2ccccc2)c(OC(=O)c2ccccc2)c1]

AQEP-04

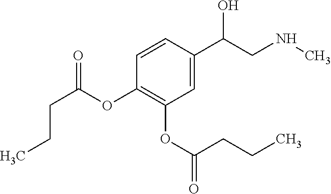 [SMILES: CCCC(=O)Oc1ccc(C(O)CNC)cc1OC(=O)CCC]

AQEP-05

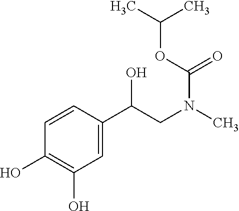 [SMILES: CC(C)OC(=O)N(C)CC(O)c1ccc(O)c(O)c1]

AQEP-06

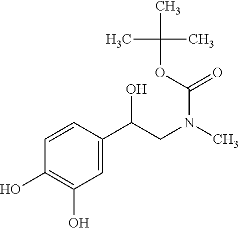 [SMILES: CN(CC(O)c1ccc(O)c(O)c1)C(=O)OC(C)(C)C]

AQEP-07

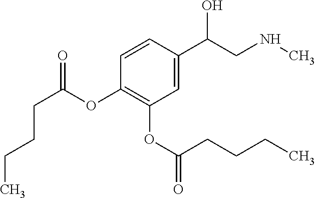 [SMILES: CCCCC(=O)Oc1ccc(C(O)CNC)cc1OC(=O)CCCC]

AQEP-08

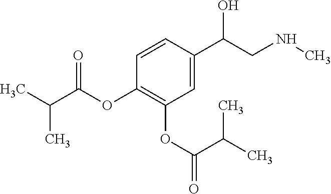 [SMILES: CNCC(O)c1ccc(OC(=O)C(C)C)c(OC(=O)C(C)C)c1]

AQEP-09

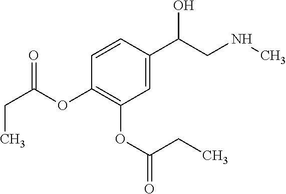 [SMILES: CCC(=O)Oc1ccc(C(O)CNC)cc1OC(=O)CC]

AQEP-10

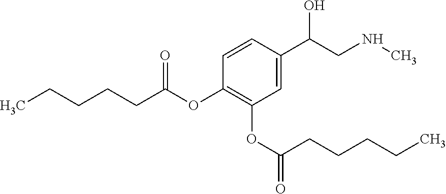 [SMILES: CCCCCC(=O)Oc1ccc(C(O)CNC)cc1OC(=O)CCCCC]

AQEP-11

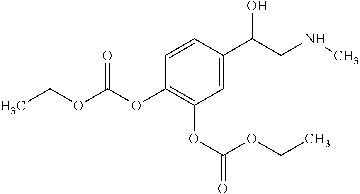 [SMILES: CCOC(=O)Oc1ccc(C(O)CNC)cc1OC(=O)OCC]

AQEP-12

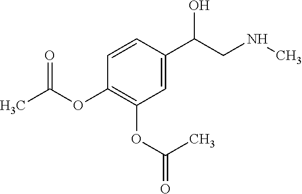 [SMILES: CNCC(O)c1ccc(OC(C)=O)c(OC(C)=O)c1]

AQEP-13

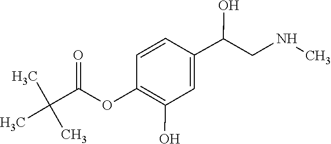 [SMILES: CNCC(O)c1ccc(OC(=O)C(C)(C)C)c(O)c1]

4-Pivaloylepinephrine

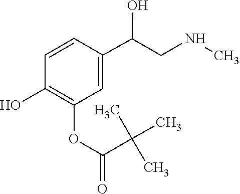 [SMILES: CNCC(O)c1ccc(O)c(OC(=O)C(C)(C)C)c1]

3-Pivaloylepinephrine
AQEP-14

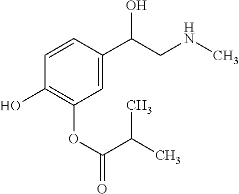 [SMILES: CNCC(O)c1ccc(O)c(OC(=O)C(C)C)c1]

3-isobutyryl epinephrine

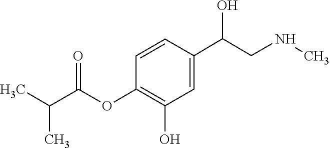 [SMILES: CNCC(O)c1ccc(OC(=O)C(C)C)c(O)c1]

4-isobutyryl epinephrine
AQEP-15

Administering epinephrine as a prodrug such as dipivefrin, or prodrugs AQEP-03, AQEP-04, AQEP-05, AQEP-06, AQEP-07, AQEP-08, AQEP-09, AQEP-10, AQEP-11, AQEP-12, AQEP-13, AQEP-14 or AQEP-15 confer certain advantages. For one, dipivefrin and prodrugs AQEP-03, AQEP-04, AQEP-05, AQEP-06, AQEP-07, AQEP-08, AQEP-09, AQEP-10, AQEP-11, AQEP-12, AQEP-13, AQEP-14 and AQEP-15 are lipophilic and therefore has a higher permeation through a mucosa. Dipivefrin and prodrugs AQEP-03, AQEP-04, AQEP-05, AQEP-06, AQEP-07, AQEP-08, AQEP-09, AQEP-10, AQEP-11, AQEP-12, AQEP-13, AQEP-14 and AQEP-15 each have a longer plasma half-life due to higher protein binding. Dipivefrin is capable of sustained blood levels, and has a reduced interaction with α-receptors, therefore minimizing or eliminating unwanted or harmful vasoconstriction. Prodrugs, for example, AQEP-09, can exhibit higher binding affinity for α- and β-receptors, with binding and activation profiles that are more similar to epinephrine than dipivefrin. Other prodrugs, and combinations of prodrugs, can exhibit binding affinities for α- and β-receptors that favor one or more receptor, similar to or different from epinephrine.

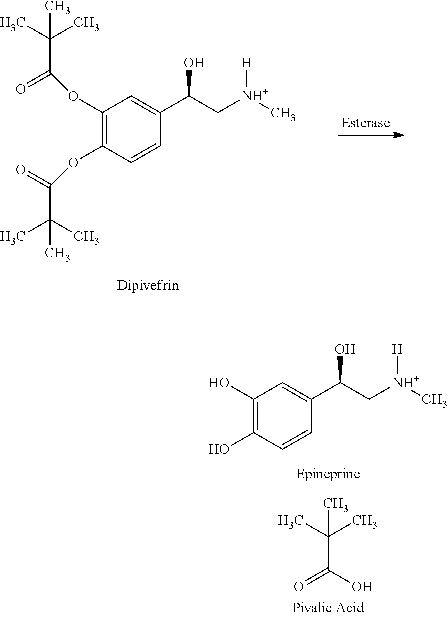

Dipivefrin or prodrugs AQEP-03, AQEP-04, AQEP-05, AQEP-06, AQEP-07, AQEP-08, AQEP-09, AQEP-10, AQEP-11, AQEP-12, AQEP-13, AQEP-14 or AQEP-15, alone or in combination, can be delivered in composition in a similar manner as with epinephrine delivered by other methods.

The compound of formula I can be a pharmaceutically acceptable salt. The pharmaceutically acceptable salt can be an acid addition salt or a base addition salt. Acid addition salts can be prepared by reacting the purified compound in its free-based form with a suitable organic or inorganic acid and isolating the salt thus formed. Examples of pharmaceutically acceptable acid addition salts include, without limitations, salts of an amino group formed with inorganic acids such as hydrochloric acid, hydrobromic acid, phosphoric ac-id, sulfuric acid and perchloric acid, or with organic acids such as acetic acid, oxalic acid, maleic acid, tartaric acid, citric acid, succinic acid or malonic acid. Base addition salts can be prepared by reacting the purified compound in its acid form with a suitable organic or inorganic base and isolating the salt thus formed. Such salts include, without limitations, alkali metal (e.g., sodium, lithium, and potassium), alkaline earth metal (e.g., magnesium and calcium), ammonium, alkylammonium, substituted alkylammonium and N⁺(C₁₋₄alkyl)₄ salts. The alkyl can be a hydroxyalkyl. Other pharmaceutically acceptable salts of the compound can include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, and valerate salts.

To deliver epinephrine, a class of prodrug compounds was tested having modifications made to the R^{1a}, R^{1b}, R² and R³ groups of epinephrine as shown below. The R^{1a} and R^{1b} groups can include esters, amides, carbonates and carbamates, orthoesters or acetals. The groups can include for example, alkyl esters, chloroalkyl esters, amides, alkyl amides, chloroalkyl amides. The R² groups can include benzylic alcohol modification. The R³ group can include amine modification or oxazolidines. An ideal prodrug would have one or more of the following attributes, is biologically acceptable, penetrates a tissue, is stable and converts in the body, tissue or blood. In some cases, the prodrug may not need any permeation enhancers at all but rather permeate sufficiently by itself. The conversion of the prodrug to active is not predictable based on chain length of the R^{1a}, R^{1b}, R² and R³ groups. In particular, a tertiary group at the second atom of the R^{1a}, R^{1b}, R² or R³ group. The permeation of the prodrug is also unpredictable based on the R^{1a}, R^{1b}, R² and R³ groups.

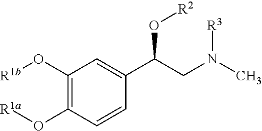

In general, a method of treating a medical condition can include administering a prodrug from a matrix, the prodrug being converted at controlled rate, for example, at a rate of 20 pg/ml to about 40 ng/ml of active compound. The prodrug can be converted at a rate where the active compound cannot be detected in plasma. For example, the prodrug conversion to active compound can be slow and sustained. In certain circumstances, the prodrug conversion to active can be less than 240 minutes, less than 180 minutes, less than 120 minutes, less than 60 minutes, or less than 30 minutes. In other circumstances, the prodrug conversion can be a slow conversion, for example, providing active compound exposure for 4 hours, 6 hours, 8 hours, 12 hours, 18 hours, 24 hours, or longer. For example, the composition can supply active compound for daily, 24 hour dosing.

In certain circumstances, the composition can be administered using microneedles or nanofibers. This type of administration can regulate delivery rates of the prodrug. In other circumstances, this type of administration can be used to direct the prodrug to specific structures in the skin tissue or other tissues.

The prodrug can be converted to 200 pg/ml to about 1200 pg/ml of active compound in less than 120 minutes. In certain embodiments, prodrug is converted to 200 pg/ml to about 1200 pg/ml of active compound in less than 100 minutes. The prodrug can also be converted to 200 pg/ml to about 600 pg/ml of active compound in less than 60 minutes. In certain embodiments, the prodrug is converted to 200 pg/ml to about 600 pg/ml of active compound in less than 45 minutes. In certain embodiments, the prodrug is converted to 200 pg/ml to about 600 pg/ml of active compound in less than 30 minutes.

In certain embodiments, the prodrug converts to create a sustained concentration of 200 pg/ml to about 600 pg/ml of active compound.

In certain embodiments, less than 100% of the prodrug is converted. In other embodiments, 100% of the prodrug is converted.

In general, a method of treating a medical condition comprising administering a prodrug, the prodrug being converted to produce a concentration of active from 20 pg/ml to about 40 ng/ml of active compound in less than 240 minutes and in which 100% of prodrug is converted. In certain circumstances, a method of treating a medical condition comprising administering a prodrug from a matrix, the prodrug being converted to produce a concentration of active from 20 pg/ml to about 40 ng/ml of active compound in less than 240 minutes and in which less than 100% of prodrug is converted. The prodrug can be administered from a matrix.

In certain embodiments, the prodrug can produce therapeutic levels over 100 pg/ml of epinephrine for a duration of at least 1 hour. In certain embodiments, the prodrug can produce therapeutic levels over 100 pg/ml of epinephrine for a duration of at least 2 hours. In certain embodiments, the prodrug produces therapeutic levels over 100 pg/ml of epinephrine for a duration of at least 3 hours. In certain embodiments, the prodrug produces therapeutic levels over 100/ml pg of epinephrine for a duration of at least 4 hours.

A prodrug can be metabolized, for example by hydrolysis. Metabolism can occur through enzymatic conversion, for example through hydrolytic enzymes, which convert a prodrug into an active compound. A prodrug can be converted at various times and in various ways in the body. A prodrug can be designed based on a targeted approach for in any suitable manner based on where and when conversion is desired. In some instances, prodrug conversion can occur systemically (e.g. in circulation). In some situations, prodrug conversion occurs intracellularly (e.g., antiviral nucleoside analogs, lipid-lowering statins). In some situations, prodrug conversion can occur extracellularly, for examples in digestive fluids or other extracellular body fluids).

In certain embodiments, at least half of the administered prodrug is converted in less than 240 minutes. In certain embodiments, at least half of the administered prodrug is converted in less than 120 minutes. In other embodiments, at least half of the administered prodrug is converted in less than 60 minutes. In other embodiments, at least half of the administered prodrug is converted in less than 30 minutes. In other embodiments, at least half of the administered prodrug is converted in less than 15 minutes. In other embodiments, at least half of the administered prodrug is converted in less than 10 minutes. In other embodiments, at least half of the administered prodrug is converted in less than 5 minutes. In other embodiments, at least half of the administered prodrug is converted in less than 1 minute.

In certain embodiments, a prodrug can be designed to convert to produce a concentration of active compound of between 20 pg/ml to about 40 ng/ml in a period of less than 120 minutes. The prodrug can be designed to convert to produce a concentration of active compound of between 20 pg/ml to about 40 ng/ml in a period of less than 60 minutes. A prodrug can be designed to convert to produce a concentration of active compound of between 20 pg/ml to about 40 ng/ml in a period of less than 30 minutes. The prodrug can be designed to convert to produce a concentration of active compound of between 20 pg/ml to about 40 ng/ml in a period of less than 15 minutes. The prodrug can be designed to convert to produce a concentration of active compound of between 20 pg/ml to about 40 ng/ml in a period of less than 10 minutes. The prodrug can be designed to convert to produce a concentration of active compound of between 20 pg/ml to about 40 ng/ml in a period of less than 5 minutes. The prodrug can be designed to convert to produce a concentration of active compound of between 20 pg/ml to about 40 ng/ml in a period of less than 1 minute.

In certain embodiments, a pharmaceutically active component can include epinephrine. In other embodiments, a prodrug can include dipivefrin. In certain embodiments, a topical pharmaceutical composition can include a skin permeability enhancer. The skin permeability enhancer can be a solvent and solubilizer. The skin permeability enhancer can be an ether, such as a monoethyl ether. In certain embodiments, the skin permeability enhancer can be diethylene glycol monoethyl ether, 2-(2-Ethoxyethoxy)ethanol, CARBITOL™, Diethylene glycol ethyl ether, Ethyldiglycol, or TRANSCUTOL®. In certain embodiments, a topical pharmaceutical composition can include an ester.

The solubility of a novel topical pharmaceutical composition can be modelled and tested using Hansen solubility parameters.

$${( Ra )}^{2} = 4 {( {δ}_{d 2} - {δ}_{d 1} )}^{2} + {( {δ}_{p 2} - {δ}_{p 1} )}^{2} + {( {δ}_{h 2} - {δ}_{h 1} )}^{2}$$

$$RED = Ra / {R}_{0}$$

### Relative Energy Difference (RED)

RED<1 The molecules are alike and will dissolve
RED=1 The system will partially dissolve
RED>1 The system will not dissolve

In certain embodiments, a pharmaceutical composition has a suitable nontoxic, nonionic alkyl glycoside having a hydrophobic alkyl group joined by a linkage to a hydrophilic saccharide in combination with a tissue delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; and (k) a stabilizing delivery vehicle, carrier, mucoadhesive, support or complex-forming species with which the compound is effectively combined, associated, contained, encapsulated or bound resulting in stabilization of the compound for enhanced delivery, wherein the formulation of the compound with the delivery-enhancing agents provides for increased bioavailability of the compound in blood plasma of a subject. Penetration enhancers have been described in J. Nicolazzo, et al., J of Controlled Disease, 105 (2005) 1-15, which is incorporated by reference in its entirety.

A chemical penetration enhancer, or absorption promoter, is a substance added to a pharmaceutical formulation in order to increase the membrane permeation or absorption rate of the coadministered drug, without damaging the membrane and/or causing toxicity or compromising the membrane for permeation followed by membrane restoration or damage reversal. There have been many studies investigating the effect of chemical penetration enhancers on the delivery of compounds across the skin, nasal mucosa, and intestine. In recent years, more attention has been given to the effect of these agents on the permeability of skin tissue. Since permeability across skin tissue can be considered to be a passive diffusion process, the steady state flux (Jss) should increase with increasing donor chamber concentration (CD) according to Fick's first law of diffusion.

Fatty acids have been shown to enhance the permeation of a number of drugs through the skin, and this has been shown by differential scanning calorimetry and Fourier transform infrared spectroscopy to be related to an increase in the fluidity of intercellular lipids.

Additionally, pretreatment with ethanol has been shown to enhance the permeability of tritiated water and albumin across ventral tongue mucosa, and to enhance caffeine permeability across porcine buccal mucosa. There are also several reports of the enhancing effect of Azone® on the permeability of compounds through oral mucosa. Further, chitosan, a biocompatible and biodegradable polymer, has been shown to enhance drug delivery through various tissues, including the intestine and nasal mucosa.

Oral transmucosal drug delivery (OTDD) is the administration of pharmaceutically active agents through the oral mucosa to achieve systemic effects. Permeation pathways and predictive models for OTDD are described, e.g. in M. Sattar, Oral transmucosal drug delivery—Current status and future prospects, Int'l. Journal of Pharmaceutics, 47(2014) 498-506, which is incorporated by reference in its entirety. OTDD continues to attract the attention of academic and industrial scientists. Despite limited characterization of the permeation pathways in the oral cavity compared with skin and nasal routes of delivery, recent advances in our understanding of the extent to which ionized molecules permeate the buccal epithelium, as well as the emergence of new analytical techniques to study the oral cavity, and the progressing development of in silico models predictive permeation, prospects are encouraging.

In order to deliver broader classes of drugs across a tissue surface, reversible methods of reducing the barrier potential of this tissue should be employed. This requisite has fostered the study of penetration enhancers that will safely alter the permeability restrictions of the a tissue surface. It has been shown that a tissue surface penetration can be improved by using various classes of transmucosal and transdermal penetration enhancers such as bile salts, surfactants, fatty acids and their derivatives, chelators, cyclodextrins and chitosan. Among these chemicals used for the drug permeation enhancement, bile salts are the most common.

In vitro studies on enhancing effect of bile salts on the permeation of compounds is discussed in Sevda Senel, Drug permeation enhancement via buccal route: possibilities and limitations, Journal of Controlled Release 72 (2001) 133-144, which is incorporated by reference in its entirety. That article also discusses recent studies on the effects of epithelial permeability of dihydroxy bile salts, sodium glycodeoxycholate (SGDC) and sodium taurodeoxycholate (TDC) and tri-hydroxy bile salts, sodium glycocholate (GC) and sodium taurocholate (TC) at 100 mM concentration including permeability changes correlated with the histological effects. Fluorescein isothiocyanate (FITC), morphine sulfate were each used as the model compound.

Chitosan has also been shown to promote absorption of small polar molecules and peptide/protein drugs through nasal mucosa in animal models and human volunteers. Other studies have shown an enhancing effect on penetration of compounds across the intestinal mucosa and cultured Caco-2 cells.

The permeation enhancer can be a phytoextract. A phytoextract can be an essential oil or composition including essential oils extracted by distillation of the plant material. In certain circumstances, the phytoextract can include synthetic analogues of the compounds extracted from the plant material (i.e., compounds made by organic synthesis). The phytoextract can include a phenylpropanoid, for example, phenyl alanine, eugenol, eugenol acetate, a cinnamic acid, a cinnamic acid ester, a cinnamic aldehyde, a hydrocinnamic acid, chavicol, or safrole, or a combination thereof. The phytoextract can be an essential oil extract of a clove plant, for example, from the leaf, stem or flower bud of a clove plant. The clove plant can be Syzygium aromaticum. The phytoextract can include 20-95% eugenol, including 40-95% eugenol, including 60-95% eugenol, and for example, 80-95% eugenol. The extract can also include 5% to 15% eugenol acetate. The extract can also include caryophyllene. The extract can also include up to 2.1% α-humulen. Other volatile compounds included in lower concentrations in clove essential oil can be β-pinene, limonene, farnesol, benzaldehyde, 2-heptanone and ethyl hexanoate. Other permeation enhancers may be added to the composition to improve absorption of the drug. Suitable permeation enhancers include natural or synthetic bile salts such as sodium fusidate; glycocholate or deoxycholate and their salts; fatty acids and derivatives such as sodium laurate, oleic acid, oleyl alcohol, monoolein, and palmitoylcarnitine; chelators such as disodium EDTA, sodium citrate and sodium laurylsulfate, azone, sodium cholate, sodium 5-methoxysalicylate, sorbitan laurate, glyceryl monolaurate, octoxynonyl-9, laureth-9, polysorbates, sterols, or glycerides, such as caprylocaproyl polyoxylglycerides, e.g., Labrasol. The permeation enhancer can include phytoextract derivatives and/or monolignols. The permeation enhancer can also be a fungal extract.

Some natural products of plant origin have been known to have a vasodilatory effect. There are several mechanisms or modes by which plant-based products can evoke vasodilation. For review, see McNeill J. R. and Jurgens, T. M., Can. J. Physiol. Pharmacol. 84:803-821 (2006), which is incorporated by reference in its entirety. Specifically, vasorelaxant effects of eugenol have been reported in a number of animal studies. See, e.g., Lahlou, S., et al., J. Cardiovasc. Pharmacol. 43:250-57 (2004), Damiani, C. E. N., et al., Vascular Pharmacol. 40:59-66 (2003), Nishijima, H., et al., Japanese J. Pharmacol. 79:327-334 (1998), and Hume W. R., J. Dent Res. 62(9):1013-15 (1983), each of which is incorporated by reference in its entirety. Calcium channel blockade was suggested to be responsible for vascular relaxation induced by a plant essential oil, or its main constituent, eugenol. See, Interaminense L. R. L. et al., Fundamental & Clin. Pharmacol. 21: 497-506 (2007), which is incorporated by reference in its entirety.

Fatty acids can be used as inactive ingredients in drug preparations or drug vehicles. Fatty acids can also be used as formulation ingredients due to their certain functional effects and their biocompatible nature. Fatty acid, both free and as part of complex lipids, are major metabolic fuel (storage and transport energy), essential components of all membranes and gene regulators. For review, see Rustan A. C. and Drevon, C. A., Fatty Acids: Structures and Properties, Encyclopedia of Life Sciences (2005), which is incorporated by reference in its entirety. There are two families of essential fatty acids that are metabolized in the human body: ω-3 and ω-6 polyunsaturated fatty acids (PUFAs). If the first double bond is found between the third and the fourth carbon atom from the ω carbon, they are called ω-3 fatty acids. If the first double bond is between the sixth and seventh carbon atom, they are called ω-6 fatty acids. PUFAs are further metabolized in the body by the addition of carbon atoms and by desaturation (extraction of hydrogen). Linoleic acid, which is a ω-6 fatty acid, is metabolized to γ-linolenic acid, dihomo-γ-linolinic acid, arachidonic acid, adrenic acid, tetracosatetraenoic acid, tetracosapentaenoic acid and docosapentaenoic acid. α-linolenic acid, which is a ω-3 fatty acid is metabolized to octadecatetraenoic acid, eicosatetraenoic acid, eicosapentaenoic acid (EPA), docosapentaenoic acid, tetracosapentaenoic acid, tetracosahexaenoic acid and docosahexaenoic acid (DHA).

It has been reported that fatty acids, such as palmitic acid, oleic acid, linoleic acid and eicosapentaenoic acid, induced relaxation and hyperpolarization of porcine coronary artery smooth muscle cells via a mechanism involving activation of the Na⁺K⁺-APTase pump and the fatty acids with increasing degrees of cis-unsaturation had higher potencies. See, Pomposiello, S. I. et al., Hypertension 31:615-20 (1998), which is incorporated by reference in its entirety. Interestingly, the pulmonary vascular response to arachidonic acid, a metabolite of linoleic acid, can be either vasoconstrictive or vasodilative, depending on the dose, animal species, the mode of arachidonic acid administration, and the tones of the pulmonary circulation. For example, arachidonic acid has been reported to cause cyclooxygenase-dependent and -independent pulmonary vasodilation. See, Feddersen, C. O. et al., J. Appl. Physiol. 68(5):1799-808 (1990); and see, Spannhake, E. W., et al., J. Appl. Physiol. 44:397-495 (1978) and Wicks, T. C. et al., Circ. Res. 38:167-71 (1976), each of which is incorporated by reference in its entirety.

Many studies have reported effects of EPA and DHA on vascular reactivity after being administered as ingestible forms. Some studies found that EPA-DHA or EPA alone suppressed the vasoconstrictive effect of norepinephrine or increased vasodilatory responses to acetylcholine in the forearm microcirculation. See, Chin, J. P. F, et al., Hypertension 21:22-8 (1993), and Tagawa, H. et al., J Cardiovasc Pharmacol 33:633-40 (1999), each of which is incorporated by reference in its entirety. Another study found that both EPA and DHA increased systemic arterial compliance and tended to reduce pulse pressure and total vascular resistance. See, Nestel, P. et al., Am J. Clin. Nutr. 76:326-30 (2002), which is incorporated by reference in its entirety. Meanwhile, a study found that DHA, but not EPA, enhanced vasodilator mechanisms and attenuates constrictor responses in forearm microcirculation in hyperlipidemic overweight men. See, Mori, T. A., et al., Circulation 102:1264-69 (2000), which is incorporated by reference herein. Another study found vasodilator effects of DHA on the rhythmic contractions of isolated human coronary arteries in vitro. See Wu, K.-T. et al., Chinese J. Physiol. 50(4):164-70 (2007), which is incorporated by reference in its entirety.

The adrenergic receptors (or adrenoceptors) are a class of G protein-coupled receptors that are a target of catecholamines, especially norepinephrine (noradrenaline) and epinephrine (adrenaline). Epinephrine (adrenaline) interacts with both α- and β-adrenoceptors, causing vasoconstriction and vasodilation, respectively. Although a receptors are less sensitive to epinephrine, when activated, they override the vasodilation mediated by β-adrenoceptors because there are more peripheral al receptors than β-adrenoceptors. The result is that high levels of circulating epinephrine cause vasoconstriction. At lower levels of circulating epinephrine, β-adrenoceptor stimulation dominates, producing vasodilation followed by decrease of peripheral vascular resistance. The α1-adrenoreceptor is known for smooth muscle contraction, mydriasis, vasoconstriction in the skin, mucosa and abdominal viscera and sphincter contraction of the gastrointestinal (GI) tract and urinary bladder. The α1-adrenergic receptors are member of the G_{q} protein-coupled receptor superfamily. Upon activation, a heterotrimeric G protein, G_{q}, activates phospholipase C (PLC). The mechanism of action involves interaction with calcium channels and changing the calcium content in a cell. For review, see Smith R. S. et al., Journal of Neurophysiology 102(2): 1103-14 (2009), which is incorporated by reference in its entirety. Many cells possess these receptors.

In general, catecholamines are synthesized from the amino acid L-tyrosine according to the following sequence: tyrosine→dopa (dihydroxyphenylalanine)→dopamine→norepinephrine (noradrenaline)→epinephrine (adrenaline). Prodrugs of epinephrine can include precursor compounds or derivatives thereof, for example, of the compounds in this synthetic sequence. For example, tyrosine, dopa(dihydroxyphenylalanine), dopamine, or norepinephrine, or esters thereof, can be C1-C22, or C3-C22 ester derivatives.

The initial step in catecholamine synthesis is the conversion of tyrosine into L-2,4-dihydroxyphenylalanine (DOPA) which then undergoes decarboxylation into dopamine. Dopamine then undergoes hydroxylation into noradrenaline which is then converted into adrenaline in the cytoplasm of the chromaffin cells. William F. Young, Chapter 16—Endocrine Hypertension, Williams Textbook of Endocrinology (12th Edition), W.B. Saunders, 2011, Pages 545-577, which is incorporated by reference in its entirety.

Tyrosine hydroxylase (TH) uses molecular oxygen, tyrosine, and biopterin as substrates to convert the amino acid L-tyrosine into 3,4-dihydroxyphenylalanine (L-DOPA). TH is the rate-limiting enzyme for catecholamine biosynthesis. DOPA decarboxylase (DDC) is pyridoxine-dependent enzyme converts L-DOPA into dopamine (DA). This step occurs so quickly that it's hard to measure L-DOPA in the brain without first inhibiting DDC. DA can be secreted into the bloodstream or undergo further hydroxylation to become norepinephrine (noradrenaline). Norepinephrine can then be further hydroxylated and a methyl group added to become epinephrine (adrenaline).

L-Dopa is well known for its role in the treatment of parkinsonism, but its biological importance lies in the fact that it is a precursor of dopamine, a neurotransmitter widely distributed in the central nervous system, including the basal ganglia of the brain (groups of nuclei within the cerebral hemispheres that collectively control muscle tone, inhibit movement, and control tremour). A deficiency of dopamine in these ganglia leads to parkinsonism, and this deficiency is at least partially alleviated by the administration of L-dopa.

Under ordinary circumstances, more epinephrine than norepinephrine is released from the adrenal medulla. In contrast, more norepinephrine is released from the sympathetic nervous system elsewhere in the body. In physiological terms, a major action of the hormones of the adrenal medulla and the sympathetic nervous system is to initiate a rapid, generalized fight-or-flight response. This response, which may be triggered by a fall in blood pressure or by pain, physical injury, abrupt emotional upset, or hypoglycemia, is characterized by an increased heart rate (tachycardia), anxiety, increased perspiration, tremour, and increased blood glucose concentrations (due to glycogenolysis, or breakdown of liver glycogen). These actions of catecholamines occur in concert with other neural or hormonal responses to stress, such as increases in adrenocorticotropic hormone (ACTH) and cortisol secretion. Britannica, The Editors of Encyclopaedia. “catecholamine”. Encyclopedia Britannica, 12 Jun. 2024, www.britannica.com/science/catecholamine. Accessed 25 Jun. 2024 which is incorporated by reference in its entirety.

Catecholamines are synthesized in the brain, in the adrenal medulla, and by some sympathetic nerve fibres. The particular catecholamine that is synthesized by a nerve cell, or neuron, depends on which enzymes are present in that cell. For example, a neuron that has only the first two enzymes (tyrosine hydroxylase and dopa decarboxylase) used in the sequence will stop at the production of dopamine and is called a dopaminergic neuron (i.e., upon stimulation, it releases dopamine into the synapse). In the adrenal medulla the enzyme that catalyzes the transformation of norepinephrine to epinephrine is formed only in the presence of high local concentrations of glucocorticoids from the adjacent adrenal cortex; chromaffin cells in tissues outside the adrenal medulla are incapable of synthesizing epinephrine.

The tissue responses to different catecholamines depend on the fact that there are two major types of adrenergic receptors (adrenoceptors) on the surface of target organs and tissues. The receptors are known as alpha-adrenergic and beta-adrenergic receptors, or alpha receptors and beta receptors, respectively. In general, activation of alpha-adrenergic receptors results in the constriction of blood vessels, contraction of uterine muscles, relaxation of intestinal muscles, and dilation of the pupils. Activation of beta-adrenergic receptors increases heart rate and stimulates cardiac contraction (thereby increasing cardiac output), dilates the bronchi (thereby increasing air flow into and out of the lungs), dilates the blood vessels, and relaxes the uterus. Drugs that block the activation of beta receptors (beta blockers), such as propranolol, are often given to patients with tachycardia, high blood pressure, or chest pain (angina pectoris). These drugs are contraindicated in patients with asthma because they worsen bronchial constriction.

The physiopharmacology of adenoreceptors ore adrenergic receptors (AR) are classified in three major types: alpha 1 (α1), alpha 2 (α2), and beta (ß)-adrenergic receptors, and each further divided into three subtypes. α1-adrenergic receptors can be a main receptor for fatty acids. For example, saw palmetto extract (SPE), widely used for the treatment of benign prostatic hyperplasia (BPH), has been reported to bind α1-adrenergic, muscarinic and 1,4-dihydropyridine (1,4-DHP) calcium channel antagonist receptors. See, Abe M., et al., Biol. Pharm. Bull. 32(4) 646-650 (2009), and Suzuki M. et al., Acta Pharmacologica Sinica 30:271-81 (2009), each of which is incorporated by reference herein. SPE includes a variety of fatty acids including lauric acid, oleic acid, myristic acid, palmitic acid and linoleic acid. Lauric acid and oleic acid can bind noncompetitively to α1-adrenergic, muscarinic and 1,4-DHP calcium channel antagonist receptors.

In human tissue distribution, the α1A subtypes are distributed in the cerebral cortex, cerebellum, heart, liver, predominant subtype in prostate and urethra and lymphocytes. The physiological functions of this subtype include contraction of urethral smooth muscle, contraction of skeletal muscle resistance arteries and contraction of human subcutaneous arteries. The main transduction mechanisms involve G_{q}/G₁₁ (phospholipase C stimulation and calcium channel). The α1B subtypes are distributed in the spleen and kidney, somatic arteries and veins, endothelial cells, lymphocytes, and osteoblasts. The physiological functions of this subtype include contraction of arteries and veins and osteoblast proliferation. Scanzano and Cosentino, Adrenergic Regulation of Innate Immunity: A Review, Frontiers in Pharmacology, 2015, 6:171, 1-18, which is incorporated by reference in its entirety.

In human tissue distribution, the α1D subtypes are distributed in the cerebral cortex, aorta, blood vessels of prostate, human bladder, and lymphocytes. The physiological functions of this subtype include contraction of arteries and ureteral contraction.

In human tissue distribution, the α2A subtypes are distributed in the brain, spleen, kidney, aorta, lung, skeletal muscle, heart and liver. The physiological functions of this subtype include presynaptic inhibition of noradrenaline release, hypotension, sedation, analgesia and hypothermia.

In human tissue distribution, the α2B subtypes are distributed in the kidney, liver, brain, lung, heart, skeletal muscle, aorta and spleen. The physiological functions of this subtype include vasoconstriction.

In human tissue distribution, the α2C subtypes are distributed in the brain, kidney, spleen, aorta, heart, liver, lung and skeletal muscle. The physiological functions of this subtype include presynaptic inhibition of noradrenaline release.

In human tissue distribution, the ß1 subtypes are distributed in the brain, lung, spleen, heart, kidney, liver and muscle. The physiological functions of this subtype include increasing cardiac output (heart rate, contractility, automaticity, conduction), renin release from juxtaglomerular cells, and lipolysis in adipose tissue.

In human tissue distribution, the ß2 subtypes are distributed in the brain, lung, lymphocytes, skin, liver and heart. The physiological functions of this subtype include smooth muscle relaxation, striated muscle tremor, glycogenolysis, increased mass and contraction speed, increase of cardiac output, increase of aqueous humor production in the eye, dilation of the arteries, glycogenolysis and gluconeogenesis in liver, insulin secretion, and bronchodilation.

In human tissue distribution, the ß3 subtypes are distributed in the adipose tissue, gall bladder, small intestine, stomach, prostate, left atrium, bladder, brown adipose tissue and endothelium of coronary microarteries. The physiological functions of this subtype include lipolysis, thermogenesis, relaxation of myometrium and colonic smooth muscle cells, vasodilation of coronary arteries, and negative cardiac entropic effect.

In certain embodiments, a permeation enhancer can be an adrenergic receptor interacter. An adrenergic receptor interacter refers to a compound or substance that modifies and/or otherwise alters the action of an adrenergic receptor. For example, an adrenergic receptor interacter can prevent stimulation of the receptor by increasing, or decreasing their ability to bind. Such interacters can be provided in either short-acting or long-acting forms. Certain short-acting interacters can work quickly, but their effects last only a few hours. Certain long-acting interacters can take longer to work, but their effects can last longer. The interacter can be selected and/or designed based on, e.g., on one or more of the desired delivery and dose, active pharmaceutical ingredient, permeation modifier, permeation enhancer, matrix, and the condition being treated. An adrenergic receptor interacter can be an adrenergic receptor blocker. The adrenergic receptor interacter can be a terpene (e.g. volatile unsaturated hydrocarbons found in the essential oils of plants, derived from units of isoprenes) or a C3-C22 alcohol or acid, preferably a C7-C18 alcohol or acid. In certain embodiments, the adrenergic receptor interacter can include farnesol, linoleic acid, arachidonic acid, docosahexanoic acid, eicosapentanoic acid, and/or docosapentaenoic acid. The acid can be a carboxylic acid, phosphoric acid, sulfuric acid, hydroxamic acid, or derivatives thereof. The derivative can be an ester or amide. For example, the adrenergic receptor interacter can be a fatty acid or fatty alcohol.

The C3-C22 alcohol or acid can be an alcohol or acid having a straight C3-C22 hydrocarbon chain, for example a C3-C22 hydrocarbon chain optionally containing at least one double bond, at least one triple bond, or at least one double bond and one triple bond; said hydrocarbon chain being optionally substituted with C₁₋₄ alkyl, C₂₋₄ alkenyl, C₂₋₄ alkynyl, C₁₋₄ alkoxy, hydroxyl, halo, amino, nitro, cyano, C₃₋₅ cycloalkyl, 3-5 membered heterocycloalkyl, monocyclic aryl, 5-6 membered heteroaryl, C₁₋₄ alkylcarbonyloxy, C₁₋₄ alkyloxycarbonyl, C₁₋₄ alkylcarbonyl, or formyl; and further being optionally interrupted by —O—, —N(R^{a})—, —N(R^{a})—C(O)—O—, —O—C(O)—N(R^{a})—, —N(R^{a})—C(O)—N(R^{b})—, or —O—C(O)—O—. Each of R^{a} and R^{b}, independently, is hydrogen, alkyl, alkenyl, alkynyl, alkoxy, hydroxylalkyl, hydroxyl, or haloalkyl.

Fatty acids with a higher degree of unsaturation are effective candidates to enhance the permeation of drugs. Unsaturated fatty acids showed higher enhancement than saturated fatty acids, and the enhancement increased with the number of double bonds. See, A. Mittal, et al. Status of Fatty Acids as Skin Penetration Enhancers—A Review, Current Drug Delivery, 2009, 6, pp. 274-279, which is incorporated by reference herein. Position of double bond also affects the enhancing activity of fatty acids. Differences in the physicochemical properties of fatty acid which originate from differences in the double bond position most likely determine the efficacy of these compounds as skin penetration enhancers. Skin distribution increases as the position of the double bond is shifted towards the hydrophilic end. It has also been reported that fatty acid which has a double bond at an even number position more rapidly effects the perturbation of the structure of both the stratum corneum and the dermis than a fatty acid which has double bond at an odd number position. Cis-unsaturation in the chain can tend to increase activity.

An adrenergic receptor interacter can be a terpene. Hypotensive activity of terpenes in essential oils has been reported. See, Menezes I. A. et al., Z. Naturforsch. 65c:652-66 (2010), which is incorporated by reference herein. In certain embodiments, the permeation enhancer can be a sesquiterpene. Sesquiterpenes are a class of terpenes that consist of three isoprene units and have the empirical formula C₁₅H₂₄. Like monoterpenes, sesquiterpenes may be acyclic or contain rings, including many unique combinations. Biochemical modifications such as oxidation or rearrangement produce the related sesquiterpenoids. In certain embodiments, the adrenergic receptor interactor can be an active drug moiety, or a derivative or prodrug thereof, for example, an ester.

An adrenergic receptor interacter can be an unsaturated fatty acid such as linoleic acid. In certain embodiments, the permeation enhancer can be farnesol. Farnesol is a 15-carbon organic compound which is an acyclic sesquiterpene alcohol, which is a natural dephosphorylated form of farnesyl pyrophosphate. Under standard conditions, it is a colorless liquid. It is hydrophobic, and thus insoluble in water, but miscible with oils. Farnesol can be extracted from oils of plants such as citronella, neroli, cyclamen, and tuberose. It is an intermediate step in the biological synthesis of cholesterol from mevalonic acid in vertebrates. It has a delicate floral or weak citrus-lime odor and is used in perfumes and flavors. It has been reported that farnesol selectively kills acute myeloid leukemia blasts and leukemic cell lines in preference to primary hemopoietic cells. See, Rioja A. et al., FEBS Lett 467 (2-3): 291-5 (2000), which is incorporated by reference herein. Vasoactive properties of farnesyl analogues have been reported. See, Roullet, J.-B., et al., J. Clin. Invest., 1996, 97:2384-2390, which is incorporated by reference herein. Both Farnesol and N-acetyl-S-trans, trans-farnesyl-L-cysteine (AFC), a synthetic mimic of the carboxyl terminus of farnesylated proteins inhibited vasoconstriction in rat aortic rings.

In certain embodiments, an interacter can be an aporphine alkaloid. For example, an interacter can be a dicentrine.

In general, an interacter can also be a vasodilator or a therapeutic vasodilator. Vasodilators are drugs that open or widen blood vessels. They are typically used to treat hypertension, heart failure and angina, but can be used to treat other conditions as well, including glaucoma for example. Some vasodilators that act primarily on resistance vessels (arterial dilators) are used for hypertension, and heart failure, and angina; however, reflex cardiac stimulation makes some arterial dilators unsuitable for angina. Venous dilators are very effective for angina, and sometimes used for heart failure, but are not used as primary therapy for hypertension. Vasodilator drugs can be mixed (or balanced) vasodilators in that they dilate both arteries and veins and therefore can have wide application in hypertension, heart failure and angina. Some vasodilators, because of their mechanism of action, also have other important actions that can in some cases enhance their therapeutic utility or provide some additional therapeutic benefit. For example, some calcium channel blockers not only dilate blood vessels, but also depress cardiac mechanical and electrical function, which can enhance their antihypertensive actions and confer additional therapeutic benefit such as blocking arrhythmias.

Vasodilator drugs can be classified based on their site of action (arterial versus venous) or by mechanism of action. Some drugs primarily dilate resistance vessels (arterial dilators; e.g., hydralazine), while others primarily affect venous capacitance vessels (venous dilators; e.g., nitroglycerine). Many vasodilator drugs have mixed arterial and venous dilator properties (mixed dilators; e.g., alpha-adrenoceptor antagonists, angiotensin converting enzyme inhibitors), such as phentolamine.

It is more common, however, to classify vasodilator drugs based on their primary mechanism of action. The figure to the right depicts important mechanistic classes of vasodilator drugs. These classes of drugs, as well as other classes that produce vasodilation, include: alpha-adrenoceptor antagonists (alpha-blockers); Angiotensin converting enzyme (ACE) inhibitors; Angiotensin receptor blockers (ARBs); beta₂-adrenoceptor agonists (β₂-agonists); calcium-channel blockers (CCBs); centrally acting sympatholytics; direct acting vasodilators; endothelin receptor antagonists; ganglionic blockers; nitrodilators; phosphodiesterase inhibitors; potassium-channel openers; renin inhibitors.

In general, the active or inactive components or ingredients can be substances or compounds that create an increased blood flow or flushing of the tissue to enable a modification or difference (increase or decrease) in uptake of the API(s), and/or have a positive or negative heat of solution which are used as aids to modify (increase or decrease) uptake.

### Sequence of Permeation Enhancer(s) and Active Pharmaceutical Ingredient(s)

The arrangement, order, or sequence of penetration enhancer(s) and active pharmaceutical ingredient(s)(API(s)) delivered to the desired surface can vary in order to deliver a desired pharmacokinetic profile. For example, one can apply the permeation enhancer(s) first by a composition, by swab, spray, gel, rinse or by a first layer of a composition then apply the API(s) by single composition, swab, spray, gel, rinse, or by a second layer of a composition. The sequence can be reversed or modified, for example, by applying the API(s) first by composition, by swab, or by a first layer of a composition, and then applying the permeation enhancer(s) by a composition, by swab, spray, gel, rinse or by a second layer of a composition, swab, spray, gel or rinse. In another embodiment, one may apply a permeation enhancer(s) by a composition, and a drug by a different composition. For example, the permeation enhancer(s) composition positioned under a composition containing the API(s), or the composition containing the API(s) positioned under a composition containing the permeation enhancer(s), depending on the desired pharmacokinetic profile.

For example, the penetration enhancer(s) can be used as a pretreatment alone or in combination with at least one API to precondition the tissue for further absorption of the API(s). The treatment can be followed by another treatment with neat penetration enhancer(s) to follow the at least one API application. The pretreatment can be applied as a separate treatment (film, gel, solution, foam, shampoo, soap, cream, ointment, emulsion or swab etc.) or as a layer within a multilayered composition construction of one or more layers. Similarly, the pretreatment may be contained within a distinct domain of a single composition, designed to dissolve and release to the tissue prior to release of the secondary domains with or without penetration enhancer(s) or API(s). The active ingredient may then be delivered from a second treatment, alone or in combination with additional penetration enhancer(s). There may also be a tertiary treatment or domain that delivers additional penetration enhancer(s) and/or at least one API(s) or prodrug(s), either at a different ratio relative to each other or relative to the overall loading of the other treatments. This allows a custom pharmacokinetic profile to be obtained. In this way, the product may have single or multiple domains, with penetration enhancer(s) and API(s) that can vary in tissue application order, composition, concentration, or overall loading that leads to the desired absorption amounts and/or rates that achieve the intended pharmacokinetic profile and/or pharmacodynamic effect.

The pharmaceutical composition can be a chewable or gelatin based dosage form, spray, gum, gel, cream, ointment, emulsion, soap, shampoo, foam, tablet, liquid or film. The composition can include textures, for example, at the surface, such as microneedles or micro-protrusions. Recently, the use of micron-scale needles in increasing skin permeability has been shown to significantly increase transdermal delivery, including and especially for macromolecules. Most drug delivery studies have emphasized solid microneedles, which have been shown to increase skin permeability to a broad range of molecules and nanoparticles in vitro. In vivo studies have demonstrated delivery of oligonucleotides, reduction of blood glucose level by insulin, and induction of immune responses from protein and DNA vaccines. For such studies, needle arrays have been used to pierce holes into skin to increase transport by diffusion or iontophoresis or as drug carriers that release drug into the skin from a microneedle surface coating. Hollow microneedles have also been developed and shown to microinject insulin to diabetic rats. To address practical applications of microneedles, the ratio of microneedle fracture force to skin insertion force (i.e. margin of safety) was found to be optimal for needles with small tip radius and large wall thickness. Microneedles inserted into the skin of human subjects were reported as painless. Together, these results suggest that microneedles represent a promising technology to deliver therapeutic compounds into the skin for a range of possible applications. Using the tools of the microelectronics industry, microneedles have been fabricated with a range of sizes, shapes and materials. Microneedles can be, for example, polymeric, microscopic needles that deliver encapsulated drugs in a minimally invasive manner, but other suitable materials can be used.

Topical routes of administration can include transdermal delivery and can also involve administration to a body surface, such as the skin, or mucous membranes. Many forms of topical administration involve applying a therapeutic agent directly to the skin; inhalable mediations, eye drops, nasal sprays, and ear drops also are considered topical administration forms. The dosage forms listed above can be tailored to directly provide systemic delivery, for example, as transdermal, nasal, skin, or inhaled compositions.

Formulations for topical application can take the compositional form of a liquid, a semisolid dosage form (e.g., a paste, a cream, a lotion, a powder, emulsion, shampoo, foam, an ointment or a gel) or a patch.

Topical films and topical patches can be provided in multiple forms including single and multi-layer drug-in-adhesive forms, matrix forms, and reservoir forms, address several of the shortcomings of semisolid formulations, for example, reducing the need for repeated application, providing accurate, and controlled release of active agent, and reducing the likelihood of unintentional removal or transfer of drug or active agent via contact with objects or other persons, but have a finite size and shape. Because topical patches have a finite size and shape, the application area is determined by the dimensions of the patch rather than the dimensions of the affected site. Accordingly, it may be necessary to use a number of patches in order to cover a large affected site. Furthermore, topical patches typically lack sufficient flexibility to be effectively administered to joints or other areas of skin subject to significant stretching movements. Topical patches can also lead to user discomfort, particular in warmer climates, and can be aesthetically unpleasing, which can also lead to poor user compliance.

Several therapeutic formulations in composition form have been described. Those that involve thin compositions on substrates of finite size and shape similar to patches inherit the same disadvantages as for patches, e.g., having the application area determined by the composition dimensions rather than the dimensions of the affected site.

The described delivery system, which provides a drug delivery system for controlled delivery of an active agent comprising a pharmaceutical composition that dries to a film form and a means for applying the film, overcomes these shortcomings. The composition in film form has the following advantageous properties: it is long-lasting, i.e., it remains in place over the administration site for the desired time, can be removed by peeling without leaving a substantial residue, is effective to achieve minimum effective concentration (MEC) of the active agent in the layers of the skin while the level of the active in systemic circulation is below therapeutic levels, is nonstaining regardless of the staining properties of the active, and can be applied to an affected site of any size.

Applicants have found that microneedles could be used to enhance the delivery of drugs through the dermal tissue, particularly with the claimed compositions. The microneedles create micron sized pores in the tissue surface which can enhance the delivery of drugs across the tissue. Solid, hollow or dissolving microneedles can be fabricated out of suitable materials including, but not limited to, metal, polymer, glass and ceramics. The microfabrication process can include photolithography, silicon etching, laser cutting, metal electroplating, metal electro polishing and molding. Microneedles could be solid which is used to pretreat the tissue and are removed before applying the composition. The drug loaded polymer composition described in this application can be used as the matrix material of the microneedles itself. These compositions can have microneedles or micro protrusions fabricated on their surface which will dissolve after forming microchannels in the tissue through which drugs can permeate.

The term “film” can include films and sheets, in any shape, including rectangular, square, or other desired shape. A film can be any desired thickness and size. In preferred embodiments, a film can have a thickness and size such that it can be administered to a user, for example, placed into the oral cavity of the user. A film can have a relatively thin thickness of from about 0.0025 mm to about 0.250 mm, or a film can have a somewhat thicker thickness of from about 0.250 mm to about 1.0 mm. For some films, the thickness may be even larger, i.e., greater than about 1.0 mm or thinner, i.e., less than about 0.0025 mm. A film can be a single layer or a film can be multi-layered, including laminated or multiple cast films. A permeation enhancer and pharmaceutically active component can be combined in a single layer, each contained in separate layers, or can each be otherwise contained in discrete regions of the same dosage form. In certain embodiments, the pharmaceutically active component contained in the polymeric matrix can be dispersed in the matrix. In certain embodiments, the permeation enhancer being contained in the polymeric matrix can be dispersed in the matrix.

Dissolving compositions can fall into three main classes: fast dissolving, moderate dissolving and slow dissolving. Dissolving compositions can also include a combination of any of the above categories. Fast dissolving compositions can dissolve in about 1 second to about 30 seconds, including more than 1 second, more than 5 seconds, more than 10 seconds, more than 20 seconds, and less than 30 seconds. Moderate dissolving compositions can dissolve in about 1 to about 30 minutes in the mouth including more than 1 minute, more than 5 minutes, more than 10 minutes, more than 20 minutes or less than 30 minutes, and slow dissolving compositions can dissolve in more than 30 minutes in the mouth. As a general trend, fast dissolving compositions can include (or consist of) low molecular weight hydrophilic polymers (e.g., polymers having a molecular weight between about 1,000 to 9,000 daltons, or polymers having a molecular weight up to 200,000 daltons). In contrast, slow dissolving compositions generally include high molecular weight polymers (e.g., having a molecular weight in millions). Moderate dissolving compositions can tend to fall in between the fast and slow dissolving compositions.

It can be preferable to use compositions that are moderate dissolving compositions. Moderate dissolving compositions can dissolve rather quickly, but also have a good level of adhesion. Moderate dissolving compositions can also be flexible, quickly wettable, and are typically non-irritating to the user. Such moderate dissolving compositions can provide a quick enough dissolution rate, most desirably between about 1 minute and about 20 minutes, while providing an acceptable adhesion level such that the composition is not easily removable once it is placed in the oral cavity of the user. This can ensure delivery of a pharmaceutically active component to a user.

A pharmaceutical composition can include one or more pharmaceutically active components. The pharmaceutically active component can be a single pharmaceutical component or a combination of pharmaceutical components. The pharmaceutically active component can be an anti-inflammatory analgesic agent, a steroidal anti-inflammatory agent, an antihistamine, a local anesthetic, a bactericide, a disinfectant, a vasoconstrictor, a hemostatic, a chemotherapeutic drug, an antibiotic, a keratolytic, a cauterizing agent, an antiviral drug, an antirheumatic, an antihypertensive, a bronchodilator, an anticholinergic, an anti-anxiety drug, an antiemetic compound, a hormone, a peptide, a protein or a vaccine. The pharmaceutically active component can be the compound, pharmaceutically acceptable salt of a drug, a prodrug, a derivative, a drug complex or analog of a drug. The term “prodrug” refers to a biologically inactive compound that can be metabolized in the body to produce a biologically active drug. For example, the pharmaceutically active component can be an ester of epinephrine, for example, dipivefrin. See, e.g., J. Anderson, et al., Site of ocular hydrolysis of a prodrug, dipivefrin, and a comparison of its ocular metabolism with that of the parent compounds, epinephrine, Invest., Ophthalmol. Vis. Sci. July 1980. The time it takes to convert 50% of the prodrug by an enzyme or multiple enzymes to yield neat epinephrine systemically in humans is referred to as the half-life. In certain embodiments, the half-life is less than 1 minute.

For example, when the prodrug is an ester of epinephrine is delivered to the systemic circulation, the half-life of hydrolysis of the ester to form epinephrine in-vivo in a human subject can be less than one minute. For example, a review of pharmacokinetic data from a study in which 12 mg of an ester of epinephrine is delivered by a sublingual composition as described herein, epinephrine plasma levels clearly indicate significant systemic absorption of epinephrine, however, the requisite parent prodrug concentration was undetectable. The absence of detectable prodrug in the plasma indicates a conversion half life of the prodrug that is not able to be calculated by traditional pharmacokinetic techniques. The detection limit of the prodrug was 2 pg/ml. By inferring the time between blood sampling timepoints (5 minutes) around the observed Cₘₐₓ, it can be reasonably concluded that the half life must be less than 1 minute and is very likely to be less than 30 seconds based on the data, in order to adequately clear the requisite prodrug level before the next timepoint.

The half-life of hydrolysis of a prodrug or an ester of epinephrine in-vivo in a human subject should not be confused with the release or dissolution of epinephrine hydrochloride from a film into 500 mL of simulated saliva as described in Alayoubi et al. Pharm. Dev. Technol. 2017, 22(8), 1012-1016. Note also that the Alayoubi report did not investigate the ability of a composition to deliver epinephrine in vivo and did not involve a prodrug or the hydrolysis of any prodrug systemically.

In some embodiments, more than one pharmaceutically active component may be included in the film, solution, paste, a cream, a lotion, a powder, emulsion, an ointment, shampoo, spray, or a gel or a patch. The pharmaceutically active components can be ace-inhibitors, anti-anginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, amphetamines, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, uterine relaxants, anti-obesity drugs, erythropoietic drugs, anti-asthmatics, cough suppressants, mucolytics, DNA and genetic modifying drugs, diagnostic agents, imaging agents, dyes, or tracers, and combinations thereof.

Suitable indications can include acne, pemphigus, alopecia areata, psoriasis, atopic dermatitis, Raynaud's phenomenon, epidermolysis bullosa, rosacea, scleroderma, hidradenitis suppurativa (acne inversa), vitiligo, ichthyosis, or pachyonychia congenital.

For example, the pharmaceutically active component can be buprenorphine, naloxone, acetaminophen, riluzole, clobazam, Rizatriptan, propofol, methyl salicylate, monoglycol salicylate, aspirin, mefenamic acid, flufenamic acid, indomethacin, diclofenac, alclofenac, diclofenac sodium, ibuprofen, ketoprofen, naproxen, pranoprofen, fenoprofen, sulindac, fenclofenac, clidanac, flurbiprofen, fentiazac, bufexamac, piroxicam, phenylbutazone, oxyphenbutazone, clofezone, pentazocine, mepirizole, tiaramide hydrochloride, hydrocortisone, predonisolone, dexarnethasone, triamcinolone acetonide, fluocinolone acetonide, hydrocortisone acetate, predonisolone acetate, methylpredonisolone, dexamethasone acetate, betamethasone, betamethasone valerate, flumetasone, fluorometholone, beclomethasone diproprionate, fluocinonide, edaravone, lurasidone, esomeprazole, lumateperone, naldmedine, doxylamine, pyridoxine, diphenhydramine hydrochloride, diphenhydramine salicylate, diphenhydramine, chlorpheniramine hydrochloride, chlorpheniramine maleate isothipendyl hydrochloride, tripelennamine hydrochloride, promethazine hydrochloride, methdilazine hydrochloride dibucaine hydrochloride, dibucaine, lidocaine hydrochloride, lidocaine, benzocaine, p-buthylaminobenzoic acid 2-(die-ethylamino) ethyl ester hydrochloride, procaine hydrochloride, tetracaine, tetracaine hydrochloride, chloroprocaine hydrochloride, oxyprocaine hydrochloride, mepivacaine, cocaine hydrochloride, piperocaine hydrochloride, dyclonine, dyclonine hydrochloride, thimerosal, phenol, thymol, benzalkonium chloride, benzethonium chloride, chlorhexidine, povidone iodide, cetylpyridinium chloride, eugenol, trimethylammonium bromide, naphazoline nitrate, tetrahydrozoline hydrochloride, oxymetazoline hydrochloride, phenylephrine hydrochloride, tramazoline hydrochloride, thrombin, phytonadione, protamine sulfate, aminocaproic acid, tranexamic acid, carbazochrome, carbaxochrome sodium sulfanate, rutin, hesperidin, sulfamine, sulfathiazole, sulfadiazine, homosulfamine, sulfisoxazole, sulfisomidine, sulfamethizole, nitrofurazone, penicillin, meticillin, oxacillin, cefalotin, cefalordin, erythromcycin, lincomycin, tetracycline, chlortetracycline, oxytetracycline, metacycline, chloramphenicol, kanamycin, streptomycin, gentamicin, bacitracin, cycloserine, salicylic acid, podophyllum resin, podolifox, cantharidin, chloroacetic acids, silver nitrate, protease inhibitors, thymadine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir, heparin, insulin, LHRH, TRH, interferons, oligonuclides, calcitonin, octreotide, omeprazone, fluoxetine, ethinylestradiol, amiodipine, paroxetine, enalapril, lisinopril, leuprolide, prevastatin, lovastatin, norethindrone, risperidone, olanzapine, albuterol, hydrochlorothiazide, pseudoephridrine, warfarin, terazosin, cisapride, ipratropium, busprione, methylphenidate, levothyroxine, zolpidem, levonorgestrel, glyburide, benazepril, medroxyprogesterone, clonazepam, ondansetron, losartan, quinapril, nitroglycerin, midazolam versed, cetirizine, doxazosin, glipizide, vaccine hepatitis B, salmeterol, sumatriptan, triamcinolone acetonide, goserelin, beclomethasone, granisteron, desogestrel, alprazolam, estradiol, nicotine, interferon beta 1A, cromolyn, fosinopril, digoxin, fluticasone, bisoprolol, calcitril, captorpril, butorphanol, clonidine, premarin, testosterone, sumatriptan, clotrimazole, bisacodyl, dextromethorphan, nitroglycerine, nafarelin, dinoprostone, nicotine, bisacodyl, goserelin, and granisetron. In certain embodiments, the pharmaceutically active component can be epinephrine, a prodrug of epinephrine, or a benzodiazepine such as diazepam or lorazepam or alprazolam.

A prodrug can be a derivative of one of the active components described herein. For example, the prodrug can be a C1-C16 acyl, alkyl aminocarbonyl, alkyloxycarbonyl, phenacyl, sulfate or phosphate derivative of the active component. For example, the prodrug can be an ester. The ester can be an ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl ester.

### Epinephrine/Dipivefrin Examples

In one example, a composition including epinephrine or its salts or esters (such as dipivefrin) can have a biodelivery profile similar to that of epinephrine administered by injection, for example, using an EpiPen. Epinephrine or its prodrug can be present in an amount of from about 0.01 mg to about 100 mg per dosage, for example, at a 0.1 mg, 5 mg, 10 mg, 20 mg, 30 mg, 40 mg, 50 mg, 60 mg, 70 mg, 80 mg, 90 mg or 100 mg dosage, including greater than 0.1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof. In another example, a composition including diazepam can have a biodelivery profile similar to that of a diazepam tablet or gel, or better.

Dipivefrin can be present in an amount of from about 0.5 mg to about 100 mg per dosage, for example, at a 0.5 mg, 1 mg, 5 mg, 10 mg, 20 mg, 30 mg, 40 mg, 50 mg, 60 mg, 70 mg, 80 mg, 90 mg or 100 mg dosage including greater than 1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof.

In another example, a composition (e.g., including epinephrine) can have a suitable nontoxic, nonionic alkyl glycoside having a hydrophobic alkyl group joined by a linkage to a hydrophilic saccharide in combination with a delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long chain amphipathic molecule; (vii) a hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof, (xiv) an N-acetylamino acid or salt thereof, (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; or (k) a stabilizing delivery vehicle, carrier, mucoadhesive, support or complex-forming species with which the compound is effectively combined, associated, contained, encapsulated or bound resulting in stabilization of the compound for enhanced delivery, wherein the formulation of the compound with the transmucosal delivery-enhancing agents provides for increased bioavailability of the compound in a blood plasma of a subject. The formulation can include approximately the same active pharmaceutical ingredient (API): enhancer ratio as in the other examples for epinephrine.

Administering epinephrine as a prodrug such as dipivefrin confers certain advantages. For one, dipivefrin is lipophilic and therefore has a higher permeation through a tissue. It also has a longer plasma half life due to higher protein binding. Vasoconstrictors trigger a cascade of interacting intracellular signals that concur in initiating and maintaining contractions. Each step of these signalling pathways is a possible logical site for potential therapeutic interventions to reduce or prevent vasoconstriction. In other circumstances, vasodilation can be targeted. It is capable of sustained blood levels, and does not interact with α-receptors, therefore minimizing or eliminating unwanted or harmful vasoconstriction.

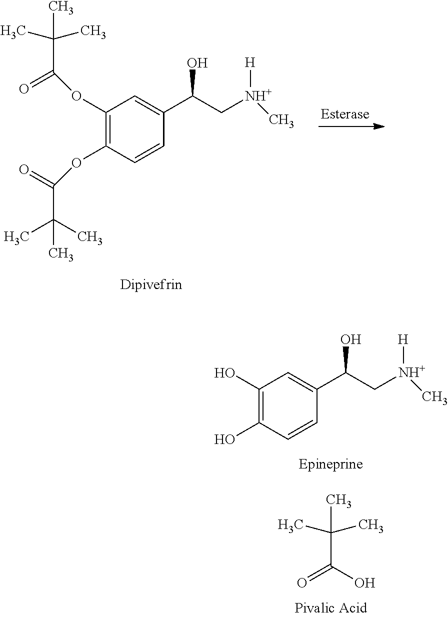

Dipifeverin can be provided as composition in a similar manner as with epinephrine.

A composition and/or its components can be water-soluble, water swellable or water-insoluble. The term “water-soluble” can refer to substances that are at least partially dissolvable in an aqueous solvent, including but not limited to water. The term “water-soluble” may not necessarily mean that the substance is 100% dissolvable in the aqueous solvent. The term “water-insoluble” refers to substances that are not dissolvable in an aqueous solvent, including but not limited to water. A solvent can include water, or alternatively can include other solvents (preferably, polar solvents) by themselves or in combination with water.

The composition can include a polymeric matrix. Any desired polymeric matrix may be used, provided that it is orally dissolvable or erodable. The dosage should have enough bioadhesion to not be easily removed and it should form a gel like structure when administered. They can be moderate-dissolving in the oral cavity and particularly suitable for delivery of pharmaceutically active components, although both fast release, delayed release, controlled release and sustained release compositions are also among the various embodiments contemplated.

A topical gel can have a peak residual adhesiveness force, which measures how sufficiently strong the gel can stay in place during the initial application. This peak residual adhesiveness force can be presented as a function of time and formulation composition. It is measured in N. In some embodiments, the peak residual adhesiveness force can be 0-0.40 N. In other embodiments, the peak residual adhesiveness force can be 0-0.40 N. In other embodiments, the peak residual adhesiveness force can be 0-0.30 N. In other embodiments, the peak residual adhesiveness force can be 0-0.20 N. In other embodiments, the peak residual adhesiveness force can be 0-0.10 N. In other embodiments, the peak residual adhesiveness force can be 0.1-0.15 N. In other embodiments, the peak residual adhesiveness force can be 0.1-0.2 N. In other embodiments, the peak residual adhesiveness force can be 0.1-0.3 N. In other embodiments, the peak residual adhesiveness force can be 0.1-0.4 N. In other embodiments, the peak residual adhesiveness force can be 0.15-0.2 N. In other embodiments, the peak residual adhesiveness force can be 0.15-0.3 N. In other embodiments, the peak residual adhesiveness force can be 0.15-0.4 N. In other embodiments, the peak residual adhesiveness force can be 0.2-0.25 N. In other embodiments, the peak residual adhesiveness force can be 0.2-0.3 N. In other embodiments, the peak residual adhesiveness force can be 0.2-0.4 N. In other embodiments, the peak residual adhesiveness force can be 0.25-0.3 N. In other embodiments, the peak residual adhesiveness force can be 0.25-0.35 N. In other embodiments, the peak residual adhesiveness force can be 0.25-0.4 N. In other embodiments, the peak residual adhesiveness force can be 0.3-0.35 N. In other embodiments, the peak residual adhesiveness force can be 0.3-0.4 N. In other embodiments, the peak residual adhesiveness force can be 0.35-0.4 N.

### Branched Polymers

The pharmaceutical composition can include dendritic polymers which can include highly branched macromolecules with various structural architectures. The dendritic polymers can include dendrimers, dendronised polymers (dendrigrafted polymers), linear dendritic hybrids, multi-arm star polymers, or hyperbranched polymers.

Hyperbranched polymers are highly branched polymers with imperfections in their structure. However they can be synthesized in a single step reaction which can be an advantage over other dendritic structures and are therefore suitable for bulk volume applications. The properties of these polymers apart from their globular structure are the abundant functional groups, intramolecular cavities, low viscosity and high solubility. Dendritic polymers have been used in several drug delivery applications. See, e.g., Dendrimers as Drug Carriers: Applications in Different Routes of Drug Administration. J Pharm Sci, VOL. 97, 2008, 123-143, which is incorporated by reference herein.

The dendritic polymers can have internal cavities which can encapsulate drugs. The steric hindrance caused by the highly dense polymer chains might prevent the crystallization of the drugs. Thus, branched polymers can provide additional advantages in formulating crystallizable drugs in a polymer matrix.

Examples of suitable dendritic polymers include poly(ether) based dendrons, dendrimers and hyperbranched polymers, poly(ester) based dendrons, dendrimers and hyperbranched polymers, poly(thioether) based dendrons, dendrimers and hyperbranched polymers, poly(amino acid) based dendrons dendrimers and hyperbranched polymers, poly(arylalkylene ether) based dendrons, dendrimers and hyperbranched polymers, poly(alkyleneimine) based dendrons, dendrimers and hyperbranched polymers, poly(amidoamine) based dendrons, dendrimers or hyperbranched polymers.

Other examples of hyperbranched polymers include poly(amines)s, polycarbonates, poly(ether ketone)s, polyurethanes, polycarbosilanes, polysiloxanes, poly(ester amine)s, poly(sulfone amine)s, poly(urea urethane)s and polyether polyols such as polyglycerols.

A composition can be produced by a combination of at least one polymer and a solvent, optionally including other components. The solvent may be water, a polar organic solvent including, but not limited to, ethanol, isopropanol, acetone, or any combination thereof. In some embodiments, the solvent may be a non-polar organic solvent, such as methylene chloride. The composition may be prepared by utilizing a selected casting or deposition method and a controlled drying process. For example, the composition may be prepared through a controlled drying processes, which include application of heat and/or radiation energy to the wet composition matrix to form a visco-elastic structure, thereby controlling the uniformity of content of the composition. The controlled drying processes can include air alone, heat alone or heat and air together contacting the top of the composition or bottom of the composition or the substrate supporting the cast or deposited or extruded composition or contacting more than one surface at the same time or at different times during the drying process. Alternatively, the compositions may be extruded.

A polymer included in the compositions may be water-soluble, water-swellable, water-insoluble, or a combination of one or more either water-soluble, water-swellable or water-insoluble polymers. The polymer may include cellulose, cellulose derivatives or gums. Specific examples of useful water-soluble polymers include, but are not limited to, polyethylene oxide, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water-insoluble polymers include, but are not limited to, ethyl cellulose, hydroxypropyl ethyl cellulose, cellulose acetate phthalate, hydroxypropyl methyl cellulose phthalate and combinations thereof. For higher dosages, it may be desirable to incorporate a polymer that provides a high level of viscosity as compared to lower dosages.

As used herein the phrase “water-soluble polymer” and variants thereof refer to a polymer that is at least partially soluble in water, and desirably fully or predominantly soluble in water, or absorbs water. Polymers that absorb water are often referred to as being water-swellable polymers. The materials useful with the present invention may be water-soluble or water-swellable at room temperature and other temperatures, such as temperatures exceeding room temperature. Moreover, the materials may be water-soluble or water-swellable at pressures less than atmospheric pressure. In some embodiments, compositions formed from such water-soluble polymers may be sufficiently water-soluble to be dissolvable upon contact with bodily fluids.

Other polymers useful for incorporation into the compositions include biodegradable polymers, copolymers, block polymers or combinations thereof. It is understood that the term “biodegradable” is intended to include materials that chemically degrade, as opposed to materials that physically break apart (i.e., bioerodable materials). The polymers incorporated in the compositions can also include a combination of biodegradable or bioerodable materials. Among the known useful polymers or polymer classes which meet the above criteria are: poly(glycolic acid) (PGA), poly(lactic acid) (PLA), polydioxanes, polyoxalates, poly(alpha-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy)propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of alpha-amino acids, copolymers of alpha-amino acids and caproic acid, copolymers of alpha-benzyl glutamate and polyethylene glycol, copolymers of succinate and poly(glycols), polyphosphazene, polyhydroxy-alkanoates or mixtures thereof. The polymer matrix can include one, two, three, four or more components.

Although a variety of different polymers may be used, it is desired to select polymers that provide mucoadhesive properties to the composition, as well as a desired dissolution and/or disintegration rate. In particular, the time period for which it is desired to maintain the composition in contact with the tissue depends on the type of pharmaceutically active component contained in the composition. Some pharmaceutically active components may only require a few minutes for delivery through the tissue, whereas other pharmaceutically active components may require up to several hours or even longer. Accordingly, in some embodiments, one or more water-soluble polymers, as described above, may be used to form the composition. In other embodiments, however, it may be desirable to use combinations of water-soluble polymers and polymers that are water-swellable, water-insoluble and/or biodegradable, as provided above. The inclusion of one or more polymers that are water-swellable, water-insoluble and/or biodegradable may provide compositions with slower dissolution or disintegration rates than compositions formed from water-soluble polymers alone. As such, the composition may adhere to the tissue for longer periods of time, such as up to several hours, which may be desirable for delivery of certain pharmaceutically active components.

Desirably, an individual dose of the composition can be a fingertip unit. Each fingertip unit can be about 0.5 grams of material. The number of fingertip units (FTUs) of application can vary depending on the part of the body to which the composition is being applied. For example, the application can be 1, 2, 3, 4, 5, 6, 7, 8, 9 or 10 FTUs. The application can be QD, BID or TID. The treatment area can be the scalp, face, neck, hand, arm, elbow, foot, leg, buttocks, knee, anterior trunk, posterior trunk, genitalia, abdomen, front of chest, or combinations thereof.

The composition can have good adhesion on the tissue of the user. Further, the prodrug should disperse and dissolve at a moderate rate, most desirably dispersing within about 1 minute and dissolving within about 3 minutes. In some embodiments, the prodrug may be capable of dispersing and dissolving at a rate of between about 1 to about 30 minutes, for example, about 1 to about 20 minutes, or more than 1 minute, more than 5 minutes, more than 7 minutes, more than 10 minutes, more than 12 minutes, more than 15 minutes, more than 20 minutes, more than 30 minutes, about 30 minutes, or less than 30 minutes, less than 20 minutes, less than 15 minutes, less than 12 minutes, less than 10 minutes, less than 7 minutes, less than 5 minutes, or less than 1 minute.

A topical gel can have a viscosity that renders it suitable for skin retention and drug penetration. The viscosity depends upon the spindle viscometer spindle speed applied during measurement. Viscosity is generally measured as centipoise (cPs), and spindle speed is given in rotations per minute (RPM). A topical gel can have a viscosity in the range of 5,000-350,000 cPs. The viscosity is typically a function of spindle speed and storage duration.

In some embodiments, the viscosity of a topical gel can be 5,000-350,000 cPs. In some embodiments, the viscosity can be 10,000-150,000 cPs at 10 RPM. In some embodiments, the viscosity of a topical gel can be 20,000-250,000 cPs at 5 RPM. In some embodiments, the viscosity of a topical gel can be 10,000-100,000 cPs. In some embodiments, the viscosity of a topical gel can be 10,000-70,000 cPs. In some embodiments, the viscosity of a topical gel can be 10,000-60,000 cPs. In some embodiments, the viscosity of a topical gel can be 10,000-50,000 cPs. In some embodiments, the viscosity of a topical gel can be 10,000-40,000 cPs. In some embodiments, the viscosity of a topical gel can be 10,000-30,000 cPs. In some embodiments, the viscosity of a topical gel can be 10,000-20,000 cPs.

In some embodiments, the viscosity of a topical gel can be 20,000-200,000 cPs. In some embodiments, the viscosity of a topical gel can be 20,000-100,000 cPs. In some embodiments, the viscosity of a topical gel can be 20,000-90,000 cPs. In some embodiments, the viscosity of a topical gel can be 20,000-80,000 cPs. In some embodiments, the viscosity of a topical gel can be 20,000-70,000 cPs. In some embodiments, the viscosity of a topical gel can be 20,000-60,000 cPs. In some embodiments, the viscosity of a topical gel can be 20,000-50,000 cPs. In some embodiments, the viscosity of a topical gel can be 20,000-40,000 cPs. In some embodiments, the viscosity of a topical gel can be 20,000-30,000 cPs.

An exemplary formulation herein was measured using a Brookfield viscometer with spindle #07 attachment.

| Brookfield viscometer | Viscosity range |
|---|---|
| spindle speed | (cPs) |
| 5 RPM | 20,000-250,000 cPs |
| 10 RPM | 10,000-150,000 cPs |

If the topical composition is applied as a spray, the viscosity ranges could be between 1-5,000 cPs, 1-4,000 cPs, 1-3,000 cPs, 1-2,000 cPs, 1-1,000 cPs, 1-500 cPs, 1-400 cPs, 1-300 cPs, 1-200 cPs or 1-100 cPs.

For instance, in some embodiments, the composition may include polyethylene oxide alone or in combination with a second polymer component. The second polymer may be another water-soluble polymer, a water-swellable polymer, a water-insoluble polymer, a biodegradable polymer or any combination thereof. Suitable water-soluble polymers include, without limitation, any of those provided above. In some embodiments, the water-soluble polymer may include hydrophilic cellulosic polymers, such as hydroxypropylmethyl cellulose, hydroxyethyl cellulose, hydroxyethylmethyl cellulose, hydroxypropyl cellulose, methylcellulose, hydroxypropyl cellulose and/or hydroxypropylmethyl cellulose. In some embodiments, one or more water-swellable, water-insoluble and/or biodegradable polymers also may be included in the polyethylene oxide-based composition. Any of the water-swellable, water-insoluble or biodegradable polymers provided above may be employed. The second polymer component may be employed in amounts of about 0% to about 80% by weight in the polymer component, more specifically about 30% to about 70% by weight, and even more specifically about 40% to about 60% by weight, including greater than 5%, greater than 10%, greater than 15%, greater than 20%, greater than 30%, greater than 40%, greater than 50%, greater than 60%, and greater than 70%, about 70%, less than 70%, less than 60%, less than 50%, less than 40%, less than 30%, less than 20%, less than 10% or less than 5% by weight.

Additives may be included in the compositions. Examples of classes of additives include preservatives, antimicrobials, excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers, anti-tacking agents, anti-static agents and mixtures thereof. These additives may be added with the pharmaceutically active component(s). As used herein, the term “stabilizer” means an excipient capable of preventing aggregation or other physical degradation, as well as chemical degradation, of the active pharmaceutical ingredient, another excipient, or the combination thereof.

Stabilizers may also be classified as antioxidants, sequestrants, pH modifiers, emulsifiers and/or surfactants, and UV stabilizers.

Antioxidants (i.e., pharmaceutically compatible compound(s) or composition(s) that decelerates, inhibits, interrupts and/or stops oxidation processes) include, in particular, the following substances: tocopherols and the esters thereof, sesamol of sesame oil, coniferyl benzoate of benzoin resin, nordihydroguaietic resin and nordihydroguaiaretic acid (NDGA), gallates (among others, methyl, ethyl, propyl, amyl, butyl, lauryl gallates), butylated hydroxyanisole (BHA/BHT, also butyl-p-cresol); ascorbic acid and salts and esters thereof (for example, acorbyl palmitate), erythorbinic acid (isoascorbinic acid) and salts and esters thereof, monothioglycerol, sodium formaldehyde sulfoxylate, sodium metabisulfite, sodium bisulfite, sodium sulfite, potassium metabisulfite, butylated hydroxyanisole, butylated hydroxytoluene (BHT), propionic acid. Typical antioxidants are tocopherol such as, for example, α-tocopherol and the esters thereof, butylated hydroxytoluene and butylated hydroxyanisole. The terms “tocopherol” also includes esters of tocopherol. A known tocopherol is α-tocopherol. The term “α-tocopherol” includes esters of α-tocopherol (for example, α-tocopherol acetate).

Sequestrants (i.e., any compounds which can engage in host-guest complex formation with another compound, such as the active ingredient or another excipient; also referred to as a sequestering agent) include calcium chloride, calcium disodium ethylene diamine tetra-acetate, glucono delta-lactone, sodium gluconate, potassium gluconate, sodium tripolyphosphate, sodium hexametaphosphate, and combinations thereof. Sequestrants also include cyclic oligosaccharides, such as cyclodextrins, cyclomannins (5 or more α-D-mannopyranose units linked at the 1,4 positions by a linkages), cyclogalactins (5 or more β-D-galactopyranose units linked at the 1,4 positions by R linkages), cycloaltrins (5 or more α-D-altropyranose units linked at the 1,4 positions by a linkages), and combinations thereof.

pH modifiers include acids (e.g., tartaric acid, citric acid, lactic acid, fumaric acid, phosphoric acid, ascorbic acid, acetic acid, succininc acid, adipic acid and maleic acid), acidic amino acids (e.g., glutamic acid, aspartic acid, etc.), inorganic salts (alkali metal salt, alkaline earth metal salt, ammonium salt, etc.) of such acidic substances, a salt of such acidic substance with an organic base (e.g., basic amino acid such as lysine, arginine and the like, meglumine and the like), and a solvate (e.g., hydrate) thereof. Other examples of pH modifiers include silicified microcrystalline cellulose, magnesium aluminometasilicate, calcium salts of phosphoric acid (e.g., calcium hydrogen phosphate anhydrous or hydrate, calcium, sodium or potassium carbonate or hydrogencarbonate and calcium lactate or mixtures thereof), sodium and/or calcium salts of carboxymethyl cellulose, cross-linked carboxymethylcellulose (e.g., croscarmellose sodium and/or calcium), polacrilin potassium, sodium and or/calcium alginate, docusate sodium, magnesium calcium, aluminium or zinc stearate, magnesium palmitate and magnesium oleate, sodium stearyl fumarate, and combinations thereof.

Examples of emulsifiers and/or surfactants include poloxamers or pluronics, polyethylene glycols, polyethylene glycol monostearate, polysorbates, sodium lauryl sulfate, polyethoxylated and hydrogenated castor oil, alkyl polyoside, a grafted water soluble protein on a hydrophobic backbone, lecithin, glyceryl monostearate, glyceryl monostearate/polyoxyethylene stearate, ketostearyl alcohol/sodium lauryl sulfate, carbomer, phospholipids, (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, and acylglutamates, quaternary ammonium salts (e.g., di-(C₁₀-C₂₄)-alkyl-dimethylammonium chloride or bromide), (C₁₀-C₂₄)-alkyl-dimethylethylammonium chloride or bromide, (C₁₀-C₂₄)-alkyl-trimethylammonium chloride or bromide (e.g., cetyltrimethylammonium chloride or bromide), (C₁₀-C₂₄)-alkyl-dimethylbenzylammonium chloride or bromide (e.g., (C₁₂-C₁₈)-alkyl-dimethylbenzylammonium chloride), N—(C₁₀-C₁₈)-alkyl-pyridinium chloride or bromide (e.g., N—(C₁₂-C₁₆)-alkyl-pyridinium chloride or bromide), N—(C₁₀-C₁₈)-alkyl-isoquinolinium chloride, bromide or monoalkyl sulfate, N—(C₁₂-C₁₈)-alkyl-polyoylaminoformylmethylpyridinium chloride, N—(C₁₂-C₁₈)-alkyl-N-methylmorpholinium chloride, bromide or monoalkyl sulfate, N—(C₁₂-C₁₈)-alkyl-N-ethylmorpholinium chloride, bromide or monoalkyl sulfate, (C₁₆-C₁₈)-alkyl-pentaoxethylammonium chloride, diisobutylphenoxyethoxyethyldimethylbenzylammonium chloride, salts of N,N-di-ethylaminoethylstearylamide and -oleylamide with hydrochloric acid, acetic acid, lactic acid, citric acid, phosphoric acid, N-acylaminoethyl-N,N-diethyl-N-methylammonium chloride, bromide or monoalkyl sulfate, and N-acylaminoethyl-N,N-diethyl-N-benzylammonium chloride, bromide or monoalkyl sulfate (in the foregoing, “acyl” standing for, e.g., stearyl or oleyl), and combinations thereof.

Examples of UV stabilizers include UV absorbers (e.g., benzophenones), UV quenchers (i.e., any compound that dissipates UV energy as heat, rather than allowing the energy to have a degradation effect), scavengers (i.e., any compound that eliminates free radicals resulting from exposure to UV radiation), and combinations thereof.

In other embodiments, stabilizers include ascorbyl palmitate, ascorbic acid, alpha tocopherol, butylated hydroxytoluene, buthylated hydroxyanisole, cysteine HCl, citric acid, ethylenediamine tetra acetic acid (EDTA), methionine, sodium citrate, sodium ascorbate, sodium thiosulfate, sodium metabi sulfite, sodium bisulfite, propyl gallate, glutathione, thioglycerol, singlet oxygen quenchers, hydroxyl radical scavengers, hydroperoxide removing agents, reducing agents, metal chelators, detergents, chaotropes, and combinations thereof. “Singlet oxygen quenchers” include, but are not limited to, alkyl imidazoles (e.g., histidine, L-camosine, histamine, imidazole 4-acetic acid), indoles (e.g., tryptophan and derivatives thereof, such as N-acetyl-5-methoxytryptamine, N-acetylserotonin, 6-methoxy-1,2,3,4-tetrahydro-beta-carboline), sulfur-containing amino acids (e.g., methionine, ethionine, djenkolic acid, lanthionine, N-formyl methionine, felinine, S-allyl cysteine, S-aminoethyl-L-cysteine), phenolic compounds (e.g., tyrosine and derivatives thereof), aromatic acids (e.g., ascorbate, salicylic acid, and derivatives thereof), azide (e.g., sodium azide), tocopherol and related vitamin E derivatives, and carotene and related vitamin A derivatives. “Hydroxyl radical scavengers” include, but are not limited to azide, dimethyl sulfoxide, histidine, mannitol, sucrose, glucose, salicylate, and L-cysteine. “Hydroperoxide removing agents” include, but are not limited to catalase, pyruvate, glutathione, and glutathione peroxidases. “Reducing agents” include, but are not limited to, cysteine and mercaptoethylene. “Metal chelators” include, but are not limited to, EDTA, EGTA, o-phenanthroline, and citrate. “Detergents” include, but are not limited to, SDS and sodium lauroyl sarcosyl. “Chaotropes” include, but are not limited to guanidinium hydrochloride, isothiocyanate, urea, and formamide. As discussed herein, stabilizers can be present in 0.0001%-50% by weight, including greater than 0.0001%, greater than 0.001%, greater than 0.01%, greater than 0.1%, greater than 1%, greater than 5%, greater than 10%, greater than 20%, greater than 30%, greater than 40%, greater than 50%, less than 50%, less than 40%, less than 30%, less than 20%, less than 10%, less than 1%, less than 0.1%, less than 0.01%, less than 0.001%, or less than 0.0001% by weight.

Useful additives can include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVA/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; or other similar polymers.

The additional components can range up to about 80%, desirably about 0.005% to 50% and more desirably within the range of 1% to 20% based on the weight of all composition components, including greater than 1%, greater than 5%, greater than 10%, greater than 20%, greater than 30%, greater than 40%, greater than 50%, greater than 60%, greater than 70%, about 80%, greater than 80%, less than 80%, less than 70%, less than 60%, less than 50%, less than 40%, less than 30%, less than 20%, less than 10%, less than 5%, about 3%, or less than 1%. Other additives can include anti-tacking, flow agents and opacifiers, such as the oxides of magnesium aluminum, silicon, titanium, etc. desirably in a concentration range of about 0.005% to about 5% by weight and desirably about 0.02% to about 2% based on the weight of all composition components, including greater than 0.02%, greater than 0.2%, greater than 0.5%, greater than 1%, greater than 1.5%, greater than 2%, greater than 4%, about 5%, greater than 5%, less than 4%, less than 2%, less than 1%, less than 0.5%, less than 0.2%, or less than 0.02%.

In certain embodiments, the composition can include plasticizers, which can include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sugar alcohols sorbitol, sodium diethylsulfosuccinate, triethyl citrate, tributyl citrate, phytoextracts, fatty acid esters, fatty acids, oils and the like, added in concentrations ranging from about 0.1% to about 40%, and desirably ranging from about 0.5% to about 20% based on the weight of the composition including greater than 0.5%, greater than 1%, greater than 1.5%, greater than 2%, greater than 4%, greater than 5%, greater than 10%, greater than 15%, about 20%, greater than 20%, less than 20%, less than 15%, less than 10%, less than 5%, less than 4%, less than 2%, less than 1%, and less than 0.5%. There may further be added compounds to improve the texture properties of the composition material such as animal or vegetable fats, desirably in their hydrogenated form. The composition can also include compounds to improve the textural properties of the product. Other ingredients can include binders which contribute to the ease of formation and general quality of the compositions. Non-limiting examples of binders include starches, natural gums, pregelatinized starches, gelatin, polyvinylpyrrolidone, methylcellulose, sodium carboxymethylcellulose, ethylcellulose, polyacrylamides, polyvinyloxoazolidone, or polyvinylalcohols.

Further potential additives include solubility enhancing agents, such as substances that form inclusion compounds with active components. Such agents may be useful in improving the properties of very insoluble and/or unstable actives. In general, these substances are doughnut-shaped molecules with hydrophobic internal cavities and hydrophilic exteriors. Insoluble and/or instable pharmaceutically active components may fit within the hydrophobic cavity, thereby producing an inclusion complex, which is soluble in water. Accordingly, the formation of the inclusion complex permits very insoluble and/or unstable pharmaceutically active components to be dissolved in water. A particularly desirable example of such agents are cyclodextrins, which are cyclic carbohydrates derived from starch. Other similar substances, however, are considered well within the scope of the present invention.

Suitable coloring agents include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide. Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, including greater than 0.001%, greater than 0.01%, greater than 0.1%, greater than 0.5%, greater than 1%, greater than 2%, greater than 5%, about 10%, greater than 10%, less than 10%, less than 5%, less than 2%, less than 1%, less than 0.5%, less than 0.1%, less than 0.01%, or less than 0.001%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, lime and grapefruit and fruit essences including apple, pear, peach, grape, strawberry, raspberry, cherry, plum, pineapple, apricot or other fruit flavors. Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof, saccharin and its various salts such as the sodium salt; dipeptide based sweeteners such as aspartame, neotame, advantame; dihydrochalcone compounds, glycyrrhizin; Stevia rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

Anti-foaming and/or de-foaming components may also be used with the compositions. These components aid in the removal of air, such as entrapped air, from the compositions. Such entrapped air may lead to non-uniform application of the composition. Simethicone is one particularly useful anti-foaming and/or de-foaming agent. The present invention, however, is not so limited and other suitable anti-foam and/or de-foaming agents may be used. Simethicone and related agents may be employed for densification purposes. More specifically, such agents may facilitate the removal of voids, air, moisture, and similar undesired components, thereby providing denser and thus more uniform application of the composition. Agents or components which perform this function can be referred to as densification or densifying agents. As described above, entrapped air or undesired components may lead to non-uniform application of the composition.

Any other optional components described in commonly assigned U.S. Pat. Nos. 7,425,292 and 8,765,167, referred to above, also may be included in the compositions described herein.

The compositions further desirably contains a buffer so as to control the pH of the composition. Any desired level of buffer may be incorporated into the composition so as to provide the desired pH level encountered as the pharmaceutically active component is released from the composition. The buffer is preferably provided in an amount sufficient to control the release from the composition and/or the absorption into the body of the pharmaceutically active component. In some embodiments, the buffer may include sodium citrate, citric acid, bitartrate salt and combinations thereof.

The pharmaceutical compositions described herein may be formed via any desired process. Suitable processes are set forth in U.S. Pat. Nos. 8,652,378, 7,425,292 and 7,357,891, which are incorporated by reference herein. In one embodiment, the composition is formed by first preparing a wet composition, the wet composition including a polymeric carrier matrix and a therapeutically effective amount of a pharmaceutically active component.

The pharmaceutical composition can adhere to a tissue surface. The present invention finds particular use in the localized treatment of body tissues, diseases, or wounds which may have moist surfaces and which are susceptible to bodily fluids, such as the mouth, the vagina, organs, or other types of tissue surfaces. The composition carries a pharmaceutical, and upon application and adherence to the tissue surface, offers a layer of protection and delivers the pharmaceutical to the treatment site, the surrounding tissues, and other bodily fluids. The composition provides an appropriate residence time for effective drug delivery at the treatment site, given the control of erosion in aqueous solution or bodily fluids such as saliva, and the slow, natural erosion of the composition concomitant or subsequent to the delivery.

The residence time of the composition depends on the erosion rate of the water erodable polymers used in the formulation and their respective concentrations. The erosion rate may be adjusted, for example, by mixing together components with different solubility characteristics or chemically different polymers, such as hydroxyethyl cellulose and hydroxypropyl cellulose; by using different molecular weight grades of the same polymer, such as mixing low and medium molecular weight hydroxyethyl cellulose; by using excipients or plasticizers of various lipophilic values or water solubility characteristics (including essentially insoluble components); by using water soluble organic and inorganic salts; by using crosslinking agents such as glyoxal with polymers such as hydroxyethyl cellulose for partial crosslinking; or by post-treatment irradiation or curing, which may alter the physical state of the composition, including its crystallinity or phase transition, once obtained. These strategies might be employed alone or in combination in order to modify the erosion kinetics of the composition. Upon application, the pharmaceutical composition adheres to the tissue surface and is held in place. Water absorption softens the composition, thereby diminishing the foreign body sensation. As the composition rests on the tissue surface, delivery of the drug occurs. Residence times may be adjusted over a wide range depending upon the desired timing of the delivery of the chosen pharmaceutical and the desired lifespan of the carrier. Generally, however, the residence time is modulated between about a few seconds to about a few days. Preferably, the residence time for most pharmaceuticals is adjusted from about 5 seconds to about 24 hours. More preferably, the residence time is adjusted from about 5 seconds to about 30 minutes. In addition to providing drug delivery, once the composition adheres to the tissue surface, it also provides protection to the treatment site, acting as an erodable bandage. Lipophilic agents can be designed to slow down erodability to decrease disintegration and dissolution.

It is also possible to adjust the kinetics of erodability of the composition by adding excipients which are sensitive to enzymes such as amylase, very soluble in water such as water soluble organic and inorganic salts. Suitable excipients may include the sodium and potassium salts of chloride, carbonate, bicarbonate, citrate, trifluoroacetate, benzoate, phosphate, fluoride, sulfate, or tartrate. The amount added can vary depending upon how much the erosion kinetics is to be altered as well as the amount and nature of the other components in the composition.

Emulsifiers typically used in the water-based emulsions described above are, preferably, either obtained in situ if selected from the linoleic, palmitic, myristoleic, lauric, stearic, cetoleic or oleic acids and sodium or potassium hydroxide, or selected from the laurate, palmitate, stearate, or oleate esters of sorbitol and sorbitol anhydrides, polyoxyethylene derivatives including monooleate, monostearate, monopalmitate, monolaurate, fatty alcohols, alkyl phenols, allyl ethers, alkyl aryl ethers, sorbitan monostearate, sorbitan monooleate and/or sorbitan monopalmitate. The HLB factors described above can apply to selection of composition components.

The amount of pharmaceutically active component to be used depends on the desired treatment strength and the composition of the layers, although preferably, the pharmaceutical component comprises from about 0.001% to about 99%, more preferably from about 0.003 to about 75%, and most preferably from about 0.005% to about 50% by weight of the composition, including, more than 0.005%, more than 0.05%, more than 0.5%, more than 1%, more than 5%, more than 10%, more than 15%, more than 20%, more than 30%, about 50%, more than 50%, less than 50%, less than 30%, less than 20%, less than 15%, less than 10%, less than 5%, less than 1%, less than 0.5%, less than 0.05%, or less than 0.005%. The amounts of other components may vary depending on the drug or other components but typically these components comprise no more than 50%, preferably no more than 30%, and most preferably no more than 15% by total weight of the composition.

The thickness of the composition may vary, depending on the thickness of each of the layers and the number of layers. As stated above, both the thickness and amount of layers may be adjusted in order to vary the erosion kinetics. Preferably, if the composition has only two layers, the thickness ranges from 0.005 mm to 2 mm, preferably from 0.01 to 1 mm, and more preferably from 0.1 to 0.5 mm, including greater than 0.1 mm, greater than 0.2 mm, about 0.5 mm, greater than 0.5 mm, less than 0.5 mm, less than 0.2 mm, or less than 0.1 mm. The thickness of each layer may vary from 10 to 90% of the overall thickness of the layered composition, and preferably varies from 30 to 60%, including greater than 10%, greater than 20%, greater than 30%, greater than 40%, greater than 50%, greater than 70%, greater than 90%, about 90%, less than 90%, less than 70%, less than 50%, less than 40%, less than 30%, less than 20%, or less than 10%. Thus, the preferred thickness of each layer may vary from 0.01 mm to 0.9 mm, or from 0.03 to 0.5 mm.

The composition can include one or more of a numbing agent, an anesthetic agent, a moisturizing agent, an antifungal agent, an antibacterial agent, a cooling agent, a warming agent, or a vitamin. A numbing agent or anesthetic agent can include lidocaine, tetracaine, bupivacaine, prilocaine, mepivacaine, procaine, chloroprocaine, ropivacaine, dibucaine, etidocaine, or benzocaine. A moisturizing agent can include stearate, olive oil, water, glycerine, ammonia, amino acids, glucosamine, creatinine, citrate and ionic solutions such as sodium, potassium, chloride, phosphate, calcium and magnesium, il-water emulsions of varying composition and may include several esters and oils such as octyl dodecanol, hexyl decanol, oleyl alcohol, decyl oleate, isopropyl stearate, isopropyl palmitate, isopropyl myristate, hexyl laureate, and dioctyl cyclohexane, mineral oil, or hyaluronic acid. An antifungal agent can include azoles (e.g., Fluconazole, Isavuconazole, Itraconazole, Ketoconazole, Miconazole, Clortrimazole, Voriconazole, Posaconazole, Ravuconazole, etc.), polyenes (e.g., natamycin, lucensomycin, nystatin, amphotericin B, etc.), echinocandins (e.g., Cancidas), pradimicins (e.g., beanomicins, nikkomycins, sordarins, allylamines, etc.), Triclosan, Piroctone, fenpropimorph, terbinafine, or derivatives and analogs thereof. The antibacterial agent can include macrolides or ketolides such as erythromycin, azithromycin, clarithromycin, and telithromycin; beta-lactams including penicillin, cephalosporin, and carbapenems such as carbapenem, imipenem, and meropenem; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, cefiriaxone, cefoperazone, ceftazidime, cefixime, cefpodoxime, ceftibuten, cefdinir, cefpirome, cefepime, and astreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin and pazufloxacin; antibacterial sulfonamides and antibacterial sulphanilamides, including para-aminobenzoic acid, sulfadiazine, sulfisoxazole, sulfamethoxazole and sulfathalidine; aminoglycosides such as streptomycin, neomvcin, kanamycin, paromycin, gentamicin, tobramycin, amikacin, netilmicin, spectinomycin, sisomicin, dibekalin and isepamicin; tetracyclines such as tetracycline, chlortetracycline, demeclocycline, minocycline, oxytetracycline, methacycline, doxycycline; rifamycins such as rifampicin (also called rifampin), rifapentine, rifabutin, bezoxazinorifamycin and rifaximin; lincosamides such as lincomycin and clindamycin; glycopeptides such as vancomycin and teicoplanin; streptogramins such as quinupristin and daflopristin; oxazolidinones such as linezolid; polymyxin, colistin and colymycin; trimethoprim, bacitracin, or phosphonomycin.

As one skilled in the art will appreciate, when systemic delivery, e.g., transmucosal or transdermal delivery is desired, the treatment site may include any area in which the composition is capable of delivery and/or maintaining a desired level of pharmaceutical in the blood, lymph, or other bodily fluid. Typically, such treatment sites include the oral, esophageal, aural, ocular, anal, nasal, and vaginal mucosal tissue, as well as, the skin. If the skin is to be employed as the treatment site, then usually larger areas of the skin wherein movement will not disrupt the adhesion of the composition, such as the upper arm or thigh, are preferred.

In certain circumstances, topical delivery can induce a physiologic effect without generating a systemically detectable amount of active in plasma or blood.

The claimed compositions including epinephrine and its prodrug can be used as a topical treatment for conditions including alopecia, contact hypersensitivity, aging skin, pemphigus, psoriasis, pruritis, atopic dermatitis, wounds, melanoma, vitiligo, acne, or urticaria.

The pharmaceutical composition can also be used as a wound dressing. A composition to supply epinephrine to wounds can establish homeostasis, for example, stop bleeding. A prodrug of epinephrine can provide stability and extended duration of action, which can be controlled or tuned by selection of components of the composition, including the particular prodrug. For example, permeability and hydrolysis of the prodrug can extend availability of epinephirine at the site of interest (e.g., a wound site). The composition as a wound dressing can include an anti-infective agent, such as an antibiotic, a cationic polymer, salts (e.g., AlSO₄). The topical composition can be easily washed away and does not impact medical care, if needed. By offering a physical, compatible, oxygen and moisture permeable, flexible barrier which can be washed away, the composition can not only protect a wound but also deliver a pharmaceutical in order to promote healing, aseptic, scarification, to ease the pain or to improve globally the condition of the sufferer. Some of the: examples given below are well suited for an application to the skin or a wound. As one skilled in the art will appreciate, the formulation might require incorporating a specific hydrophilic/hygroscopic excipient which would help in maintaining good adhesion on dry skin over an extended period of time. Another advantage of the present invention when utilized in this manner is that if one does not wish that the composition be noticeable on the skin, then no dyes or colored substances need be used. If, on the other hand, one desires that the composition be noticeable, a dye or colored substance may be employed.

While the pharmaceutical composition can adhere to tissues, such as surfaces of the skin or wounds. The pharmaceutical composition can adhere to the skin if prior to application the skin is wet with an aqueous-based fluid such as water, saliva, wound drainage or perspiration. The composition can adhere to the skin until it erodes due to contact with water by, for example, rinsing, showering, bathing or washing. The composition may also be readily removed by peeling without significant damage to tissue. Skin is considered as an important route of administration of drugs for both local and systemic effects. The effectiveness of topical therapy depends on the physicochemical properties of the drug and adherence of the patient to the treatment regimen as well as the system's ability to adhere to skin during the therapy so as to promote drug penetration through the skin barrier. Conventional formulations for topical and dermatological administration of drugs have certain limitations like poor adherence to skin, poor permeability and compromised patient compliance. For the treatment of diseases of body tissues and wounds, the drug has to be maintained at the site of treatment for an effective period of time. Topical composition forming systems are such developing drug delivery systems meant for topical application to the skin, which adhere to the body, forming a thin transparent composition and provide delivery of the active ingredients to the body tissue. These are intended for skin application as emollient or protective and for local action or transdermal penetration of medicament for systemic action. The transparency is an appreciable feature of this polymeric system which greatly influences the patient acceptance. In the current discussion, the composition are described as a promising choice for topical and transdermal drug delivery. Further the various types of film forming systems (sprays/solutions, gels and emulsions) along with their evaluation parameters can also be applied. See, e.g., Kathe, et al., Film Forming Systems for Topical and Transdermal Delivery, Asian J Pharm Sci. 2017 November; 12(6):487-497. doi: 10.1016/j.ajps.2017.07.004. Epub 2017 Jul. 5, which is incorporated by reference in its entirety.

Referring to FIG. 1A, after application of the formulation to the skin, the composition of the film forming system can change significantly due to the loss of the volatile components of the vehicle which results in formation of residual film on the skin surface. In this process the concentration of drug can increase, reaching saturation level and with the possibility of reaching supersaturation level on the skin surface. Supersaturation can result in the enhanced drug flux through the skin by increasing the thermodynamic activity of the formulation without affecting the skin's barrier, thereby reducing the side effects or irritation.

Referring to FIG. 1B, this depicts the drug permeation pattern of various topical compositions such as transdermal patches, film forming systems, or semisolids. In case of transdermal patches the drug can be stored in a reservoir from which the drug release occurs slowly and the drug is absorbed into the capillaries from where it is transported to systemic circulation or it is formulated as a topical patch so as to penetrate the skin to reach the target tissue for localized action. An active pharmaceutical ingredient can also be incorporated into semisolids show their activity on the skin surface or penetrate into skin layers to reach the site of action but systemic delivery of drugs is limited due to various factors. Film forming systems can also function as both semisolids and patches and can provide topical as well as transdermal delivery as desired.

In certain embodiments, gel formulations can be used. Optimal gel formulations have low spreadability and adhesive forces while retaining an appropriate level of gel hardness. Gel hardness, spreadability, and adhesiveness are parameters that are critical for gel formulations.

In terms of selecting a gel preservative or stabilizer, an optimal compound can function as a preservative under mildly acidic to acidic conditions. Another advantageous parameter is an ability to demonstrate acceptable potential antimicrobial activity against only Gram+/−bacteria, molds, and yeasts. While the antimicrobial activity may be limited by the expected pH range of the topical gel platform.

### Topical Formulations

The barrier function of the skin resides in the stratum corneum (SC), which is considered to be the major barrier for drug penetration, as it is impermeable to almost all compounds and molecules with a molecular weight greater than 600 Daltons. Diffusion along the concentration gradient is the principal mechanism by which the permeation of a drug across human skin takes place. There are two general pathways for drugs to permeate the SC: the transepidermal route and the transappendageal route. Barnes, T. M.; et al. Vehicles for Drug Delivery and Cosmetic Moisturizers: Review and Comparison. Pharmaceutics 2021, 13, 2012. https://doi.org/10.3390/pharmaceutics13122012. The transepidermal route contains two micropathways; the transcellular route and the intercellular route.

The more common pathway for drugs to permeate the skin is the intercellular route. The intercellular route involves the drug diffusing around the corneocytes and through the continuous lipid matrix. The interdigitating nature of the corneocytes yields a tortuous pathway for intercellular drug permeation, which is in contrast to the relatively direct pathway of the transcellular route. It has been estimated that water has 50 times further to travel by the intercellular route than it does through the direct thickness of the SC. Small hydrophilic drug molecules generally favor the transcellular route over the intercellular route, and vice versa for lipophilic molecules.

The transappendageal, or shunt route, involves the flow of drug molecules through the sweat glands and hair follicles via the associated sebaceous glands. These skin appendages provide a continuous channel directly across the SC barrier. However, it is generally accepted that because the surface area occupied by sweat glands and hair follicles is small, typically only 0.1% of the skin's total surface area, their contribution to epidermal permeation is also usually small. Although sweat ducts provide a hydrophilic pathway across the skin due to the secretion of an aqueous salt solution, permeation may be limited as sweat moves in the reverse direction to that of the drug. In addition, sebaceous glands are filled with a lipid-rich sebum, which may present a barrier to hydrophilic drugs. Even so, the transappendageal route can be vital for ions and large polar molecules which do not freely cross the SC.

Gels tend to be thick and liquefy on contact with warm skin, providing a cooling sensation. They dry to form a thin film which does not stain or leave behind a greasy texture. These features make gels cosmetically favorable, however, they are poorly occlusive and generally do not provide hydration. Gels are both easy to apply and wash off.

Sprays are easily applied in a thin layer with little waste and good absorption and are also useful for difficult to reach areas. They may produce a cooling sensation upon application, however, they may also be associated with stinging and burning upon application. In addition, there is no risk of contamination of the unused portion of the spray, making them an excellent choice of vehicle for the delivery of an active drug that need to be kept sterile, but also applied regularly.

### Excipients for Topical Gel Formulations

Pharmaceutical excipients can encompass any compound or substance outside of the active ingredient that fulfills a vital role in a formulation. Their purpose to enhance specific characteristics, whether associated with the performance of the formulation or aspects related to patient comfort, safety, and acceptability. E.g., Arribada, et al, Excipients in drug delivery systems: A comprehensive review of approved inactive ingredients for human ophthalmic formulations, European Journal of Pharmaceutics and Biopharmaceutics, Volume 208,

2025, 114637, ISSN 0939-6411, doi.org/10.1016/j.ejpb.2025.11463. In formulating a topical composition, the composition should ideally include a synergistic blend of moisturizing agents, including humectants, emollients, and occludents, which will help to improve efficacy. A challenge is that the formulation must also be cosmetically acceptable and be one that the patient will use. As a general rule, the heavier the moisturizer the better the effect, but there is a need to balance the heaviness of a moisturizer with what the patient is willing to use. Compliance is in accordance with patient preferences and desired results, hence, will likely be poor if the patient is unsatisfied with the moisturizer. Ideally, clinicians should recommend therapeutic moisturizers that are non-comedogenic, non-irritating, and compatible with current therapeutic regimens.

Excipients can include solubility enhancers, viscosity enhancers, penetration enhancers or permeation enhancers, buffering agents (buffers, acidifying, and alkalizing agents), preservatives (antimicrobial and antioxidant agents). In gel formulations, such excipients can be used to deliver an optimal formulation that balances viscosity, loading and spreadability to enhance performance of the formulation and patient compliance.

### Viscosity Enhancers or Gelling Agents

Thickeners of gelling agents are important excipients that influence topical vehicle viscosity, skin retention, and drug penetration. Barnes, T. M.; et al., Vehicles for Drug Delivery and Cosmetic Moisturizers: Review and Comparison. Pharmaceutics 2021, 13, 2012. doi.org/10.3390/pharmaceutics13122012. They work by imparting their natural thickness to the vehicle. Naturally-derived thickeners (e.g., hydroxyethyl cellulose, guar gum, xanthan gum, gelatin) are polymers that absorb water, causing them to swell up and increase the viscosity of the vehicle. Mineral thickeners (e.g., magnesium aluminium silicate, silica, bentonite) are also natural, and like naturally derived thickeners they absorb water and oils to increase viscosity, but produce a different result to the final emulsion. The final group are synthetic thickeners (e.g., cetyl palmitate, ammonium acryloyldimethyltaurate). They are often used in lotions and creams. The most common synthetic thickener is carbomer, an acrylic acid polymer that is water-swellable and can be used to form clear gels Polymers commonly used for this purpose include cellulose and its derivatives (methylcellulose (MC), carboxy methylcellulose (CMC), hydroxypropyl methylcellulose (HPMC, or hypromellose), hydroxyethyl cellulose (HEC)), polyvinyl alcohol (PVA), carbomers, pluronic acid (poloxamer), polyvinyl pyrrolidone (PVP), hyaluronic acid (HA), xanthan gum and other polymeric gels, with these examples listed in Table 1—viscosity enhancers. These compounds, which are classified as bioadhesive hydrogels or in situ forming gels, comprising both polar and non-polar groups, undergo swelling upon water contact. Id. In addition, in situ forming systems seem to be a great strategy to improve not only ocular bioavailability, but also patient compliance, since they overcome issues such as ocular irritation provoked by ointments or lid sticking by gels. Id.

### Silicones

Silicones act as non-greasy occlusive to aid in moisture retention. They can also function as emollients, filling in spaces between desquamating corneocytes, to create a smooth skin surface that patients desire. Dimethicone and cyclomethicone are the two most common silicones used in topical vehicle formulations.

### Humectants

Humectants are hygroscopic substances that behave in a similar fashion to the natural moisturizing factor (NMF) in the skin. Humectants readily penetrate the SC and act like biological sponges by attracting and holding water in the skin, either by drawing it up from the dermis into the epidermis, or from the environment when the atmospheric humidity is >80%. They can also cause water to evaporate into the environment, and thus need to be used with occlusive agents to decrease or prevent TEWL, and help enhance epidermal barrier function and hydration. Some humectants also possess emollient properties. Many humectants are the same molecules that form the NMF, such as lactic acid, pyrrolidone carboxylic acid (PCA) and amino acids. Humectants such as glycerol, triacetin, and polyols have traditionally been included into aqueous-based formulations, such as gels to improve the moisturizing and occlusive effect gels lack in comparison to creams and ointments.

### Emollients or Spreadability Agent

Emollients simulate the intracellular bilayers of the SC. They improve the ‘feel’ of the skin by filling the spaces in between corneocytes and also provide what has been termed ‘skin slip’ or lubricity, imparting a sense of softness and plasticity. This improves the overall appearance and texture of the skin. Some common emollients include essential fatty acids (e.g., linoleic acid, stearic acid, oleic acid, fatty alcohols), which are found in various natural oils (e.g., wool fat, palm oil, coconut oil). These essential fatty acids can be oxidized to eicosanoids, which are important signaling molecules involved in inflammatory pathways and the immune system. It is therefore thought that fatty acids may also influence skin physiology.

### Stiffening Agents

Stiffening agents are the main structure-forming excipients in topical semisolid formulations, such as ointments and cream. A number of natural and synthetic lipids and hydrocarbons work as stiffening agents including white soft paraffin/petrolatum, liquid paraffin, lanolin, beeswax, carnauba wax, cetyl alcohol, and isohexadecane. Topical formulations with a high lipid content, as found in ointments and creams, form a protective occlusive barrier on the skin, protect from harmful substances, and help to keep the skin hydrated. Stiffening agents also act as emollients to smooth, soften, and lubricate the skin.

### Penetration Enhancers

Penetration-enhancing substances have the property of modifying the permeability of the epithelium and can generally be categorized as chelating agents. These agents operate through diverse mechanisms, ultimately resulting in the diminished barrier function of the epithelium. Once the promoter molecule is incorporated into the cell membrane, it disturbs the phospholipid structure of the corneal epithelium, resulting in a change in its permeability. Surfactant enhancers in low concentrations are believed to boost the permeability of drugs and peptides, either by aiding their transit through cell membranes or the transcellular pathway. When surfactant molecules are incorporated into the lipid bilayer and saturate it, they form polar defects on the cell membranes with consequent removal of the phospholipids, leading to membrane solubilization. Azones and dimethyl sulfoxide (DMSO) are also known to disrupt the lipid domains and improve the partitioning of drugs into the SC. In contrast, long chain fatty acids (e.g., oleic acid, linoleic acid) insert between the hydrophobic lipid tails to increase the fluidity of the lamellar bilayers. For example, amitriptyline, one of the topical pain medications formulated in combination with other drugs, was found to permeate 4 to 5-fold more in the presence of fatty acids such as oleic acid. Surfactants and detergents also act as penetration enhancers by solubilizing the SC lipids.

### Buffering Agents

A buffer system is composed of different compounds responsible for maintaining the acid-base balance of a solution or formulation, by resisting a change in pH when acids or bases are added. Citric acid, acetic, boric, and hydrochloric acids, sodium carbonate and borate, and citrate-based preparations, are examples of approved buffers.

### Preservatives

Pharmaceutical products, without preservation, are easily susceptible to be contaminated with mold, fungi, and bacteria, leading to spoilage and greater risk of infection to the patient. A preservative can have broad antimicrobial activity, chemical and thermal stability, compatibility with the container and the other compounds of the formulation are some of the desired characteristics of preservatives. Preservatives are usually included in topical vehicles containing water, such as aqueous gels and creams, to prevent contamination and growth of microorganisms. In non-aqueous systems, such as ointments, it is uncommon to include antimicrobial preservatives since microorganisms, while they may survive, rarely proliferate under such conditions. A preservative should be active against a wide spectrum of microorganisms and its selection should be based on several factors such as compatibility with the formulation, toxicity, irritancy potential, and the site at which the vehicle is to be applied. The concentration of preservative should also be taken into consideration since other excipients within the vehicle may have some antimicrobial activity. Examples of some commonly used preservatives include alcohols (e.g., benzyl alcohol, ethanol, phenoxyethanol), hydroxybenzoates (all salts), phenols (e.g., chlorocresol), and quaternary ammonium compounds (e.g., benzalkonium chloride, cetrimide)

### Antioxidants

Many drugs in aqueous solution are susceptible to oxidative degradation, which may be prevented by the addition of an antioxidant. The use of antioxidants can sometimes be avoided by reducing the amount of oxygen dissolved in a solution or present in the container, especially for single-use or sterile products. The inclusion of certain excipients in the topical vehicles such as fixed oils, fats, and diethyl ether-based compounds, such as Transcutol P, which may contain low level peroxides, can also accelerate drug oxidation and should be avoided for drugs prone to oxidation. Antioxidants are also occasionally included to inhibit rancidity in topical vehicles containing unsaturated oils and fats, which are common in emulsion-based formulations. Examples of commonly used antioxidants in topical formulations include alkyl gallates, butylated hydroxyanisole, butylated hydroxytoluene, ascorbyl palmitate, sodium ascorbyl phosphate, and tocopherols, where most exhibit synergistic effects when used in combination or in the presence of metal chelators such as edetic acid.

### Solvents

For aqueous-based topical formulations, such as aqueous gels and oil-in-water emulsions, water is often the main drug solvent, although various water-miscible solvents, such as polyols (e.g., polyethylene glycol and propylene glycol) and alcohols (e.g., ethanol, isopropyl alcohol, benzyl alcohol), can be included to improve drug solubility. Solvents enhance drug absorption through several mechanisms. At the site of topical application, volatile solvents, such as water, alcohol, and propellants (used in foams and sprays) evaporate, leading to enhanced drug absorption due to increased concentration. In the case of topical vehicles that are already saturated with drugs, incorporation of solvents with relatively high boiling points may help to keep the drug from precipitating over a long period of time at the site of application, facilitating the absorption process. The solvent diethylene glycol monoethyl ether (DEGEE) is currently used in over 500 cosmetic products and has enabled the formulation of a topical 5% dapsone gel for the treatment of acne. Osborne D W. Diethylene glycol monoethyl ether: an emerging solvent in topical dermatology products. J Cosmet. Dermatol. 2011 December; 10(4):324-9. doi: 10.1111/j.1473-2165.2011.00590.x. PMID: 22151944.

The following examples are provided to illustrate pharmaceutical compositions, as well as, methods of making and using, pharmaceutical compositions and devices described herein.

### EXAMPLES

### Example 1

### Modeling Dipivefrin HCl Solubility Using Hansen Solubility Parameters—Dataset Generation

A calculation of result energetic qualities for a solute API was performed. A calculation of the effective solubility space for thermodynamically favorable interactions.

| | Dipivefrin HCl, | Dipivefrin HCl, |
|---|---|---|
| Parameter | 0.5% w/w | 5.0% w/w |
| Dispersive force, δD (MPa^{1/2}) | 17.2 | 16.6 |
| Polar interaction, δP (MPa^{1/2}) | 14.4 | 12.8 |
| Hydrogen bonding, δH (MPa^{1/2}) | 15.6 | 18.5 |
| Interaction radius (R₀) | 12.6 | 13.6 |
| Data fit quality | 0.929 | 0.929 |

### Example 2 Topical Gel

An anhydrous gel was tested to allow for a water-soluble delivery platform. The solvents for the polymer base can ethyl alcohol, propylene glycol, and diethylene glycol monoethyl ether. It was determined that a single-phase platform can avoids concerns inherent in maintaining distinct oily/aqueous phases. Exemplary solvents exhibited a relative energy difference (RED) less than 1.

| | Relative Energy Difference |
|---|---|
| Solvating Excipient | (RED; <1 soluble, >1 insoluble) |
| Propylene glycol | 0.273 |
| diethylene glycol monoethyl ether | 0.623 |

### Example 3 Tissue Thickness and Permeation

Referring to FIG. 2A (through 6 hour timepoint) and FIG. 2B (through 24 hour timepoint), dermatome sectioning of the porcine ear was conducted to sample membranes of varying thickness that both capture the complete epidermis and a varying thickness of the dermis. Dermatome sectioning completed at 200 μm, 300 μm, and 400 μm to provide membranes of a varying total thickness. Ondansetron formulation used was 1% ondansetron, 1% menthol, 15% IPA, 9.3% NMP, and 73.7% citrate buffer (pH 4.0). While a thickness of 200 μm demonstrated higher total amounts of ondansetron permeated, this was probably due the inherent porosity in the membrane itself (e.g., follicular drug transport) and the lack of a significant underlying dermal layer (see table below). A tissue thickness of 400 μm demonstrated a linear relationship between the total amount of ondansetron permeated and duration.

| Tissue | Amount Ondansetron | Amount Ondansetron | |
|---|---|---|---|
| Thickness | Permeated, 6 hours | Permeated, 24 hours | Donor Cell |
| (μm) | (μg) | (μg) | Conditions |
| 200 | 21.2 (±2.5) | 542.4 (±74.3) | Empty |
| 400 | 5.7 (±3.9) | 43.8 (±19.4) | Filled |

### Example 4—Impact of Tissue Type on Permeation

As porcine tissue varies with regard to stratum corneum and epidermal thickness as a function of sampling location, multiple sampling locations have been selected for comparison. Dermatome sectioning of multiple porcine tissue samples at a constant thickness. Dermatome sectioning completed at 400 μm. Tissue types evaluated included porcine ear, shoulder, rump, and belly locations

Referring to FIG. 3A (through 400 minutes) and FIG. 3B (through 1500 minutes), the average amount of ondansetron permeated (in ug) is measured as a function of time. The permeation is measured through varying intact porcine ear tissue types at constant thickness (400 um total thickness). The Ondansetron formulation used was 1% ondansetron, 1% menthol, 15% IPA, 9.3% NMP, and 73.7% citrate buffer (pH 4.0). While porcine shoulder and belly skin sections demonstrated more rapid early permeation of ondansetron, this was probably due the inherent porosity in the membrane itself (e.g., follicular drug transport) in that t=0 minutes demonstrated the presence of ondansetron within the receiver media. Given the comparably low standard deviation and the lack of ondansetron detected in the receiver media at t=0 minutes compared to other tissue types, this tissue type has been selected for continued ex-vivo permeation method development.

### Example 5—Evaluation of Dipivefrin Topical Gel Formulation

A topical gel was prepared with varying dipivefrin concentrations (0.5 and 5.0% w/w). Both in vitro and ex vivo permeation was measured for the following formulation.

| | Topical Gel | Topical Gel |
|---|---|---|
| | Composition | Composition |
| Component | (% w/w) | (% w/w) |
| Dipivefrin HCl | 0.50 | 5.00 |
| Ethyl alcohol | 33.00 | 33.00 |
| Hydroypropyl cellulose | 2.00 | 2.00 |
| Propylene glycol | 32.25 | 30.00 |
| Diethyleneglycol monoethyl | 32.25 | 30.00 |
| ether | | |

Referring to FIGS. 4A and 4B, the in vitro permeation and flux was measured for 5.0% dipivefrin (w/w). Rapid release from the topical gel matrix was observed.

Referring to FIG. 4A, the amount permeated (in ug) was measured as a function of time (in minutes). Referring to FIG. 4B, the average flux of dipivefrin permeated through CelluSep was measured. The flux (ug/cm²*min) was measured as a function of time (minutes).

Referring to FIGS. 4C and 4D, ex vivo permeation was measured for varying dipivefrin concentrations (0.5 and 5.0% w/w) in porcine ear tissue (400 μm). Significant permeation through porcine ear tissue in combination with rapid flux was observed for topical gel containing 0.5% or 5.0% (w/w) dipivefrin HCl. Referring to FIG. 4C, this shows amount permeated (ug) as a function of time (min) over 1500 minutes. Referring to FIG. 4D, this shows flux (ug/cm2*min) as a function of time (min) over 1500 minutes.

### Example 6

Stability was evaluated for dipivefrin topical formulations in a gel assay. An exemplary formulation is provided below.

Referring to FIG. 5A, this graph shows the stability trend for a dipivefrin topical formulation assay at 25° C. over 3 months. Referring to FIG. 5B, this graph shows the stability trend for a dipivefrin topical formulation assay at 40° C. over 3 months.

Multiple dipivefrin gel formulations were evaluated. Formulations 1-3: API loading ranging from 2.5-10% w/w. Formulation 4: Reduced ethyl alcohol (50%) of original loading. Formulation 5: Reduced diethylene glycol monoethyl ether (50%) of original loading. Formulation 6: Reduced propylene glycol (50%) of original loading.

| | | | | | Dip | Dip | Dip | | | | Total |
|---|---|---|---|---|---|---|---|---|---|---|---|
| | | | | | RRT | RRT | RRT | Total Known | Total unknown | Total | (#) |
| | | | Epinephrine | PD-14 | 1.19 | 1.95 | 2.00 | Impurities | Impurities | Impurities | Impurities |
| | | | Units | | | | | | | | |
| | Stability | | % | % | % | % | % | | | | |
| Sample | Time Point | Stab | RRT | RRT | RRT | RRT | RRT | | | | |
| Batch | (M) | Condition | N/A | N/A | 1.19 | 1.95 | 2.00 | % LC | % LC | % LC | (#) |
| Dipivefrin | 0 | Initial | ND | <0.05 | 0.06 | ND | 0.07 | 0.00 | 0.13 | 0.13 | 2 |
| Gel | 1 | 40° C./75% RH | ND | 0.14 | <0.05 | <0.05 | 0.12 | 0.14 | 0.12 | 0.26 | 2 |
| 1-1-2 | 2 | | ND | 0.16 | <0.05 | <0.05 | 0.13 | 0.16 | 0.13 | 0.29 | 2 |
| | 3 | | ND | 0.18 | <0.05 | <0.05 | 0.12 | 0.18 | 0.12 | 0.30 | 2 |
| | 3 | 25° C./60% RH | ND | 0.16 | <0.05 | ND | 0.12 | 0.16 | 0.12 | 0.28 | 2 |
| Dipivefrin | 0 | Initial | ND | <0.05 | 0.10 | ND | <0.05 | 0.00 | 0.10 | 0.10 | 1 |
| Gel | 1 | 40° C./75% RH | ND | 0.15 | 0.08 | <0.05 | 0.07 | 0.15 | 0.15 | 0.30 | 3 |
| 2-1-2 | 2 | | ND | 0.17 | 0.07 | <0.05 | 0.08 | 0.17 | 0.15 | 0.32 | 3 |
| | 3 | | ND | 0.18 | 0.06 | ND | 0.09 | 0.18 | 0.15 | 0.33 | 3 |
| | 3 | 25° C./60% RH | ND | 0.14 | 0.08 | ND | 0.06 | 0.14 | 0.14 | 0.28 | 3 |
| Dipivefrin | 0 | Initial | ND | <0.05 | ND | ND | 0.10 | 0.00 | 0.10 | 0.10 | 1 |
| Gel | 1 | 40° C./75% RH | ND | 0.16 | ND | 0.06 | 0.14 | 0.16 | 0.20 | 0.36 | 3 |
| 3-1-2 | 2 | | ND | 0.16 | ND | 0.07 | 0.14 | 0.16 | 0.21 | 0.37 | 3 |
| | 3 | | ND | 0.17 | ND | 0.07 | 0.14 | 0.17 | 0.21 | 0.38 | 3 |
| | 3 | 25° C./60% RH | ND | 0.16 | ND | ND | 0.14 | 0.16 | 0.14 | 0.30 | 2 |
| Dipivefrin | 0 | Initial | ND | <0.05 | 0.07 | ND | 0.06 | 0.00 | 0.13 | 0.13 | 2 |
| Gel | | | | | | | | | | | |
| 4-1-2 | | | | | | | | | | | |
| | 1 | 40° C./75% RH | ND | 0.15 | <0.05 | <0.05 | 0.10 | 0.15 | 0.10 | 0.25 | 2 |
| | 2 | | ND | 0.12 | <0.05 | <0.05 | 0.11 | 0.12 | 0.11 | 0.23 | 2 |
| | 3 | | ND | 0.16 | <0.05 | <0.05 | 0.12 | 10.16 | 0.12 | 0.28 | 2 |
| | 3 | 25° C./60% RH | ND | 0.16 | <0.05 | <0.05 | 0.11 | 10.16 | 0.11 | 0.27 | 2 |
| Dipivefrin | 0 | Initial | ND | <0.05 | 0.07 | ND | 0.07 | 10.00 | 0.14 | 0.14 | 2 |
| Gel | | | | | | | | | | | |
| 5-1-2 | | | | | | | | | | | |
| | 1 | 40° C./75% RH | ND | 0.14 | <0.05 | ND | 0.12 | 0.14 | 0.12 | 0.26 | 2 |
| | 2 | | ND | 0.16 | <0.05 | <0.05 | 0.11 | 0.16 | 0.11 | 0.27 | 2 |
| | 3 | | ND | 0.18 | ND | <0.05 | 0.13 | 0.18 | 0.13 | 0.31 | 2 |
| | 3 | 25° C./60% RH | ND | 0.14 | <0.05 | ND | 0.11 | 0.14 | 0.11 | 0.25 | 2 |
| Dipivefrin | 0 | Initial | ND | <0.05 | 0.08 | ND | 0.08 | 0.00 | 0.16 | 0.16 | 2 |
| Gel | | | | | | | | | | | |
| 6-1-2 | | | | | | | | | | | |
| | 1 | 40° C./75% RH | ND | 0.14 | <0.05 | <0.05 | 0.12 | 0.14 | 0.12 | 0.26 | 2 |
| | 2 | | ND | 0.16 | <0.05 | ND | 0.13 | 0.16 | 0.13 | 0.29 | 2 |
| | 3 | | ND | 0.16 | ND | <0.05 | 0.14 | 0.16 | 0.14 | 0.30 | 2 |
| | 3 | 25° C./60% RH | ND | 0.16 | <0.05 | ND | 0.12 | 0.16 | 0.12 | 0.28 | 2 |

Abreviations:
ND—Not Detected
Method: ARDTM-135
Note:
Epinephrine and PD-15 determined with ARDTM-136 Units

### Example 7

Stability was evaluated for dipivefrin topical formulations by measuring degradant generation over 3 months.

Referring to FIG. 6A, this graph shows PD-14 content (00 LC) as function of time (months). This shows PD-14 content at 25° C. over 3 months.

Referring to FIG. 6B, this graph shows PD-14 content (0% LC) as function of time (months). This shows PD-14 content at 40° C. over 3 months.

Referring to FIG. 6C, shows RRT 2.00 content (00 LC) as function of time (months) at 25° C. over 3 months.

Referring to FIG. 6D, this graph shows RRT 2.00 content (00 LC) as a function of time, at 40° C. over 3 months.

### Example 8

The claimed topical formulations were tested for permeation.

Referring to FIG. 7A, a dipivefrin topical formulation was tested for permeation through porcine ear tissue. The graph shows epinephrine prodrug amount permeated (ug) as a function of time (minutes). The active pharmaceutical ingredient (API) concentration was varied from 2.5%, 5% and 10%.

Referring to FIG. 7B, a dipivefrin topical formulation was tested for permeation through porcine ear tissue for 5% API, as well as with ethyl alcohol reduction, diethylene glycol monoethyl ether reduction and propylene glycol reduction. The graph shows epinephrine prodrug amount permeated (ug) as a function of time (minutes).

Multiple topical gel formulations were evaluated:
Formulations 1-3: API loading ranging from 2.5-10% w/w.
Formulation 4: Reduced ethyl alcohol (50%) of original loading
Formulation 5: Reduced diethylene glycol monoethyl ether (50%) of original loading
Formulation 6: Reduced propylene glycol (50%) of original loading

Dose proportional increases were observed in permeation between 2.5% and 10% API loading.

| | Formulation ID and Composition (% w/w) | | | | | |
|---|---|---|---|---|---|---|
| Ingredient | 1 | 2 | 3 | 4 | 5 | 6 |
| Dipivefrin HCl | 5.00 | 10.00 | 2.50 | 5.00 | 5.00 | 5.00 |
| hydroxypropyl cellulose | 2.00 | 2.00 | 2.00 | 2.00 | 2.00 | 2.00 |
| Propylene glycol | 30.00 | 27.50 | 31.25 | 38.25 | 37.14 | 15.00 |
| diethylene glycol monoethyl ether | 30.00 | 27.50 | 31.25 | 38.25 | 15.00 | 37.14 |
| Ethyl alcohol | 33.00 | 33.00 | 33.00 | 16.50 | 40.86 | 40.86 |
| Total | 100.0 | 100.0 | 100.0 | 100.0 | 100.0 | 100.0 |

### Example 9

The stability of the topical formulation was tested in two assays. Formulation 1 was evaluated with API loading of 5% w/w. Assay trends are directed downward under accelerated conditions (40° C./75% RH)

FIG. 8A shows epinephrine prodrug content (% LC) as a function of time, trending at 250/60% RH.

FIG. 8B shows epinephrine prodrug content (% LC) as a function of time, trending at 400/75% RH.

| | Composition |
|---|---|
| Ingredient | (% w/w) |
| AQEP-09 HCl | 5.00 |
| hydroxypropyl cellulose | 2.00 |
| Propylene glycol | 30.00 |
| diethylene glycol monoethyl ether | 30.00 |
| Ethyl alcohol | 33.00 |
| Total | 100.0 |

### Example 10

Stability trends for a claimed topical formulation (AQEP-09) was evaluated by measuring degradant generation. Formulation 1 was tested with API loading of 5% (% w/w). Degradant formation trends are directed upward under accelerated conditions (40° C./75% RH) for epinephrine, PD-15, and RRT 1.80.

Referring to FIG. 9A, this shows degradant concentration (% LC) as a function of time (months), trending at 25° C./60% RH.

Referring to FIG. 9B, this shows degradant concentration (% LC) as a function of time (months), trending at 40° C./75% RH.

| | | | Imp (> or =0.05) | | | Total Impurities | | | |
|---|---|---|---|---|---|---|---|---|---|
| | | | | | PD9 | | | | Total |
| | | | | | RRT | Total Known | Total unknown | Total | (#) |
| | | | Epinephrine | PD-15 | 1.80 | Impurities | Impurities | Impurities | Impurities |
| | | | Units | | | | | | |
| | | | % | % | % | % LC | % LC | % LC | (#) |
| | | | Limit | | | | | | |
| | | | — | — | — | — | — | — | |
| Sample Batch | Stab Time | Stab | RRT | RRT | RRT | | | | |
| | Point (M) | Condition | N/A | N/A | 1.80 | | | | |
| AQEP-09 Gel | 0 | Initial | ND | 0.12 | 0.07 | 0.12 | 0.07 | 0.19 | 2 |
| 1-1-2 | 1 | 40° C./75% RH | 0.21 | 0.46 | 0.12 | 0.67 | 0.12 | 0.79 | 3 |
| | 2 | | 0.23 | 0.51 | 0.14 | 0.74 | 0.14 | 0.88 | 3 |
| | 3 | | 0.23 | 0.66 | 0.14 | 0.88 | 0.14 | 1.02 | 3 |
| | 3 | 25° C./60% RH | 0.23 | 0.51 | 0.18 | 0.73 | 0.18 | 0.91 | 3 |

Abreviations:
ND—Not Detected
Method: ARDTM-135
Note:
Epinephrine and PD-15 determined with ARDTM-136

### Example 11

The claimed topical formulation was evaluated for permeation as shown in FIG. 10. The graph shows the amount of epinephrine prodrug permeated (ug) as a function of time (minutes). Exemplary formulations were evaluated including Formulations 1-3, with API loading from 2.5-10% w/w. The results indicate that the permeation results observed are similar for both epinephrine prodrugs.

| | Composition (% w/w) | | |
|---|---|---|---|
| | Formula- | Formula- | Formula- |
| Ingredient | tion 1 | tion 2 | tion 3 |
| AQEP-09 HCl or Dipivefrin HCl | 5.00 | 10.00 | 2.50 |
| hydroxypropyl cellulose | 2.00 | 2.00 | 2.00 |
| Propylene glycol | 30.00 | 27.50 | 31.25 |
| diethylene glycol monoethyl ether | 30.00 | 27.50 | 31.25 |
| Ethyl alcohol | 33.00 | 33.00 | 33.00 |
| Total | 100.0 | 100.0 | 100.0 |

### Example 12

The claimed topical compositions were tested for permeation using both porcine ear and buccal tissue.

Referring to FIG. 11A, this graph shows topical dipivefrin gel permeation as a function of dipivefrin concentration and tissue type over time (24 hours). Differences in API permeation were observed between varying tissue types, with dose dependent permeation also being observed. Dose proportional increases in permeation were observed between 2.5% and 10% API loading. There was no difference in permeation observed across excipient reductions.

Referring to FIG. 11B, this graph shows topical dipivefrin gel permeation as a function of dipivefrin concentration and tissue type over time (6 hours). Within the first 6 hours, differences in API permeation were observed for varying tissue types (e.g., lag observed for buccal tissue). Dose dependent permeation was observed between 2.5%/5% and 10% API loading, with 2.5% and 5% API performing similarly in porcine ear tissue.

### Example 13

The claimed topical compositions were tested for flux across both porcine ear and buccal tissue.

Referring to FIG. 12, this graph shows dipivefrin gel flux as a function of dipivefrin concentration and tissue type over time (24 hours). The differences in API flux were observed between varying tissue types (e.g., lag observed for buccal tissue). Dose dependent flux was observed between 2.5%/5% and 10% API loading, with 2.5% and 5% API performing similarly within porcine ear tissue.

### Example 14—Rat and Minipig Studies

A study was conducted to evaluate the pharmacokinetics of the claimed composition in a rat model. The purpose of this study was to enable selection of dosing regimen to be used on Passive Cutaneous Anaphylaxis (PCA) model in mammalian subjects. In this study, 15 male rats were studied to model the effects of the claimed composition on passive cutaneous anaphylaxis with a topical route of exposure. Item 1 was a dipivefrin topical gel, 0% (placebo). Item 2 was dipivefrin topical gel, 1% (low dose). Item 3 was dipivefrin topical gel, 3% (high dose). The Wistar Han rat was chosen as the animal model for this study as it is an appropriate model to study the effects of drug candidates on passive cutaneous anaphylaxis, based on available literature.

| | | | | No. of | No. of |
|---|---|---|---|---|---|
| | | Dose | | Animals | Animals |
| | | volume | Dose | 30 mins | 60 mins |
| Group | | (mg) | Concen- | post | post |
| No. | Test material | per ear | tration | dosing | dosing |
| 1 | Item 1 - Placebo | 30 | 0% | — | 3 |
| 2 | Item 2 - low dose | 30 | 1% | 3 | — |
| 3 | Item 2- low dose | 30 | 1% | — | 3 |
| 4 | Item 3 - high dose | 30 | 2.5% | 3 | — |
| 5 | Item 3- high dose | 30 | 2.5% | — | 3 |

In this study, the pharmaceutical composition was applied on the dorsal side of both ears for groups 2-5 and only on the left ear of group 1 using a gloved finger until absorbed. The topical gel was applied as an even layer with no rubbing required.

| Scoring | Observations |
|---|---|
| 0 | Normal |
| 1 | Blue color at sites of injection |
| 2 | Blue color at different sites in addition to |
| | the site of injection in both ears |
| 3 | Blue color in all the ear (right and left) |
| 4 | Blue color in all the ear (right and left) |
| | and on the body of the animal |

FIG. 13B depicts the Evans blue quantification of the data in FIG. 13A. Evans blue dye (EBD) is an inert tracer that measures plasma volume in subjects and vascular permeability in animal models. EBD is non-toxic and not metabolically active in mammalian circulation. Because of its rapid binding to serum albumin and lack of cellular uptake, its plasma concentration remains relatively constant within hours following intravenous injection. Therefore, its final plasma concentration following a brief moment of circulatory distribution is used to depict the total plasma volume of test subjects, including human patients. Wang, H L., Lai, T. Optimization of Evans blue quantitation in limited rat tissue samples. Sci Rep 4, 6588 (2014). https://doi.org/10.1038/srep06588. As depicted in the graph, the Group 1 data is the naïve sample. Group 2 shows the placebo. Group 3 show the low dose (1%) data. Group 4 shows the high dose (2.5%) data. Group 5 shows the desloratadine data.

Referring to FIG. 13C, this graph depicts dipivefrin concentration in tissue (ng/g) 60 minutes post application for both low dose (1%) and high dose (2.5%) samples.

Referring to FIG. 13D, this figure shows epinephrine concentration in tissue (ng/g) 60 minutes post application for both low dose (1%) and high dose (2.5%) samples.

Referring to FIG. 13E, this figure shows epinephrine concentration in tissue (ng/g) 30 minutes post application for both low dose (1%) and high dose (2.5%) samples.

Referring to FIG. 13F, this figure shows epinephrine concentration in tissue (ng/g) 60 minutes post application for both low dose (1%) and high dose (2.5%) samples.

For each of FIG. 13C-F, the raw data is presented in the tables that follow the graphs.

Referring to FIG. 14A, this graph shows the results of prodrug dipivefrin exposure after application to rat ears. The dark gray bar represents concentration in ng/g after 30 minutes of exposure for both 1% (low dose) and 2.5% (high dose) compositions. The light gray bar represents concentration in ng/g after 60 minutes of exposure for both 1% (low dose) and 2.5% (high dose) compositions.

Referring to FIG. 14B, this graph shows the results of epinephrine exposure after application to rat ears. The dark gray bar represents concentration in ng/g after 30 minutes of exposure for both 1% (low dose) and 2.5% (high dose) compositions. The light gray bar represents concentration in ng/g after 60 minutes of exposure for both 1% (low dose) and 2.5% (high dose) compositions.

The results show that absorption and conversion of the claimed topical pharmaceutical composition having a prodrug of epinephrine at two concentrations (1% and 2.5%) were confirmed at 30 and 60 minutes after application of 50 mg of gel to the ear of rats (N=6 each). The results also showed no detectable dipivefrin or increase in epinephrine in systemic plasma. Finally, the results showed that significant and durable dipivefrin concentrations shows that epinephrine exposure is present well beyond 1 hour.

A non-clinical pharmacokinetic study was also conducted in minipigs. In this study, a 2.5% topical gel was applied at 30 mg/cm2 in 7, 10 and 12% body surface area (BSA) coverage respectively, and data was collected over 6 hours.

Referring to FIG. 14C, this graph depicts tissue concentrations of dipivefrin in the epidermis of minipigs. Referring to FIG. 14D, this graph depicts the tissue concentrations of dipivefrin in the dermis of minipigs. This shows high exposure in both dermis and epidermis lasting more than 6 hours and no systemic exposure even at high body surface area coverage.

### Example 15—Systemic Reactions

Type I hypersensitivity reactions are systemic reactions that occur in response to re-exposure to an antigen. Reactions are mediated by IgE and involve mast cells and basophils. IgE binding to the IgE receptor FcεR1 on the surface of mast cells, activates the inflammatory pathway and leads to degranulation of mast cells. This leads to the release of proinflammatory mediators such as histamine, tryptase, TNFα and IL4. The Passive Cutaneous Anaphylaxis (PC) model is dinitrophenol (DNP) specific, IgE antibody in the ear followed 24 hours later by an intravenous (IV) injection of DNP-HAS allergen which elicits a systemic allergic reaction. For testing, Item 1 was a dipivefrin topical gel, 0% (placebo). Item 2 was dipivefrin topical gel, 1% (low dose). Item 3 was dipivefrin topical gel, 3% (high dose).

The inducing agents were as follows:

| | | Vehicle for | | Vehicle for | |
|---|---|---|---|---|---|
| | Antibody | antibody | Antigen | antigen | Dye |
| Identification: | DNP-specific | PBS pH 7.4 | DNP-HSA | 1% Evans blue in | Evans Blue |
| | mouse IgE | 1x | | 0.9% saline for | |
| | | | | injection USP | |
| Alternate name: | Mouse Anti- | — | 2,4- | — | — |
| | DNP | | Dinitrophenyl- | | |
| | Recombinant | | Human Serum | | |
| | Antibody | | Albumin | | |
| | (cloneSPE7) | | | | |
| Catalog number | PABZ-033 | — | D5059 | — | — |
| Storage Conditions: | −20° C. | CRT | 2-8° C. | CRT | CRT |
| Provided by: | Test facility | Test Facility | Test Facility | Test Facility | Test Facility |
| Supplier: | Creative | — | LGC Biosearch | — | Sigma |
| | Biolabs | | | | |

The positive control and vehicle were as follows

| | Positive control | Vehicle |
|---|---|---|
| Identification: | Desloratadine | 0.5% Hydroxyethylcellulose |
| | | in UPW |
| Storage Conditions: | 2-8° C. | CRT |
| Provided by: | Test Facility | Test Facility |
| Supplier: | Caymam chemicals | — |

FIG. 15A-D depict the results on this study. Specifically, FIG. 15A shows the results for the cytokinetic impact of the treatments during PCA relative to the normal state for TNF-α. FIG. 15B shows the results for the cytokinetic impact of the treatments during PCA relative to the normal state for KC-GRO. FIG. 15C shows the results for the cytokinetic impact of the treatments during PCA relative to the normal state for IL-6. FIG. 15D shows the results for the cytokinetic impact of the treatments during PCA relative to the normal state for INF-γ. For these figures, the dark gray bar (far left) represents the placebo. The light gray bar (center) represents the low dose (1%) of the tested pharmaceutical composition. The medium gray bar (far right) represents the high dose (2.5% of the tested pharmaceutical formulation.

Referring to FIG. 15E, this figure depicts epinephrine as a potent inhibitor of inflammatory cytokines in humans. It is established that epinephrine inhibits endotoxin-induced IL-Ibeta production; roles of tumor necrosis factor-alpha and IL-10. Van Der Poll and Lowry, Am J Physiol., 1997, 273:R1829-R2137. The study shows that decreased serum concentrations of IL-1b in septic patients following treatment with epinephrine. Similar effects seen with other inflammatory cytokines. Epinephrine increases serum concentrations of the anti-inflammatory cytokine, IL-10.

The literature has shown that norepinephrine (NE) potently suppresses TNFα while inducing rapid IL-10 secretion. Brain, Behav. Immun., 2018, Didem Agac, Leonardo D. Estrada, Robert Maples, Lora V. Hooper, J. David Farrar. Referring to FIG. 15F, this shows TNF-a suppression overtime. Referring to FIG. 15G, this shows IL-10 secretion overtime. Referring to FIG. 15H, this shows percent survival as a function of time (hours post LPS challenge). Id.

### Example 16—Ex Vivo Human Skin Microdialysis

A study was performed using ex vivo human skin for microdialysis of interstitial fluid across multiple treatment groups.

Referring to FIG. 16A, this graph shows the collection tubes across different treatment groups and the flow direction. The groups include a vehicle+PBS, 2.5% dipivefrin prodrug (AQST-108)+PBS, 10% dipivefrin prodrug (AQST-108)+PBS, vehicle+anti-IgE, 2.5% dipivefrin prodrug (AQST-108)+IgE, and 10% dipivefrin prodrug (AQST-108)+IgE.

Referring to FIG. 16B, this graph shows human skin microdialysis results by depicting histamine levels (ng/ml) over time. Here, excised human skin was used for microdialysis for interstitial fluid across multiple treatment groups.

### Example 17—NK Cell Activity

Referring to FIG. 17, this graph shows modulation of natural killer (NK) cell activity after treatment with the topical dipivefrin composition. The graph shows that the pharmaceutical composition effectively deactivated NK cells across of range of concentrations exceeding the half-maximal inhibitory concentration (IC50) at 140, and 280 nM concentrations. IC50 is a measurement of how much of a drug is needed to inhibit a biological process by half. In this figure, the ritlecitnib IC50 extracted from an FDA summary basis of approval for Litfulo™, results shown are a cross-study comparison. Ritlecitnib used for the treatment of severe alopecia areata.

The following table shows a summary of human NK % CD25+CD69+ cell inactivation.

| | % inactivation relative to |
|---|---|
| Sample: | vehicle |
| Vehicle | N/A |
| Cortisol 200 nM | 39% |
| Dexamethasone 200 nM | 22% |
| Dexamethasone 1 μM | 19% |
| Pro drug 2500 nM | 66% |
| Pro drug 1000 nM | 68% |
| Pro drug 500 nM | 67% |
| Epinephrine 2500 nM | 51% |
| Epinephrine 1000 nM | 61% |
| Epinephrine 500 nM | 65% |
| Pro drug 2500/ Epinephrine 2500 nM | 17% |
| Pro drug 2500/ Epinephrine 1000 nM | 29% |
| Pro drug 2500/ Epinephrine 500 nM | 32% |
| Pro drug 500 nM/Epinephrine 500 nM | 59% |
| Pro drug 750 nM/Epinephrine 250 nM | 57% |
| Pro drug 250 nM/Epinephrine 750 nM | 56% |

### Example 18—Clinical Overview

In addition to high expression on skin tissue, adrenergic receptor beta 2 (ADRB2) has a high expression on immune cells. See Single cell type—ADRB2—The Human Protein Atlas, ADRB2—adrenergic receptor beta 2. Skin disorders are thus potentially addressable by adrenergic receptor-mediated immune cell targeting. For example, targeting neutrophils can help address to chronic granulomatous disease, and leukocyte adhesion deficiencies. Natural killer cells' increased activity is linked to alopecia areata, lupus and rheumatoid arthritis, and decreased activity is linked to viral infections, and proliferative diseases including cancers. Langerhans cell histiocytosis, skin manifestations are common and mimic other conditions. T-cells can be targeted to address autoimmune diseases such as alopecia areata, viral infections. Helper T-cells can be targeted to address atopic dermatitis, mycobacterial infections and asthma.

A two-part clinical study was conducted for alopecia areata (AA) patients. AA is an autoimmune disease leading to hair loss on the scalp, face, and in more severe cases, other body areas. The mechanisms leading to AA are multifactorial, including an autoimmune response that results in the loss of hair follicle immune privilege. The patient will be given treatment based on disease severity (>50% involvement—JAK inhibitors; <50% involvement—corticosteroids).

In Part 1, single ascending doses of a single formulation of AQST-108 were given. No serious adverse events (SAE) or topical adverse events (AE) were reported. The calculated % AQST-108 in skin remained relatively consistent, between 9-14%. No AQST-108 plasma concentrations were observed. Thus, systemic epinephrine concentrations remained within normal physiologic range for all doses. The 1.0% AQST-108 strength was down-selected to Part 2.

In Part 2, a single dose of 1 of 3 formulations of 1.0% of AQST-108 was given. No serious adverse events (SAE) or topical adverse events (AE) were reported. The calculated % AQST-108 in skin remained relatively consistent, between 11-12%. No AQST-108 plasma concentrations observed. Thus, systemic epinephrine concentrations remained within normal physiologic range for all doses.

This shows that AQST-108 is safe and can achieve a high percent of drug delivery with the current formulation. Further, the lack of plasma AQST-108 concentration implies conversion in the dermis at the targeted site of action.

### Example 19—Topical Gel Formulations

The following formulations are exemplary. In some embodiments, the % w/w dipivefrin is indicated at 1%, 2.5%, 5% and 10% respectively. Evaluation of formulation stability as a function of dipivefrin content and functional excipient loading was performed within a glass scintillation vial. Formulations containing sodium metabisulfite were not found to prevent significant reductions in dipivefrin assay content or prevent the generation of degradants >0.5% after 1 month at 40° C./75% RH. Formulations containing 5.0% citric acid and 0.5% Vitamin E prevented reductions in dipivefrin assay content and prevented the generation of degradants >0.5% for both topical gel formulations containing either 1% or 10.000 dipivefrin after 1 month at 40° C./75.5 RH.

| | | 1% | 2.5% | 5% | 10% |
|---|---|---|---|---|---|
| | | Dipivefrin | Dipivefrin | Dipivefrin | Dipivefrin |
| | | Composition | Composition | Composition | Composition |
| Ingredient | Function | (% w/w) | (% w/w) | (% w/w) | (% w/w) |
| (—) - Dipivefrin | API | 1.104 | 2.760 | 5.520 | 11.040 |
| Hydrochloride | | | | | |
| Hydroxypropylcellulose, | Gelling | 1.500 | 1.500 | 1.500 | 1.500 |
| NF, Ph. Eur., JP | polymer | | | | |
| Glycerin, USP | Solvent | 29.748 | 28.920 | 27.540 | 24.780 |
| Diethylene glycol | Permeation | 35.448 | 34.620 | 33.240 | 30.480 |
| monoethyl ether, | enhancer | | | | |
| USP/NF | | | | | |
| Ethyl Alcohol, USP | Solvent | 16.500 | 16.500 | 16.500 | 16.500 |
| Diisopropyl adipate | Spreadability | 10.000 | 10.000 | 10.000 | 10.000 |
| | agent | | | | |
| Vitamin E | Antioxidant | 0.500 | 0.500 | 0.500 | 0.500 |
| Sorbic acid, NF | Preservative | 0.200 | 0.200 | 0.200 | 0.200 |
| Citric acid, anhydrous | pH modifier | 5.000 | 5.000 | 5.000 | 5.000 |
| Total | N/A | 100.0 | 100.0 | 100.0 | 100.0 |

Multiple formulation iterations were tested for a compositional design of experiment (DOE), evaluating both chemical stability and physical attributes of a gel platform. A texture analyzer was used to quantify subjective physical attributes of gel performance including gel hardness, spreadability and adhesiveness. Optimal formulations have low spreadability and adhesive forces while retaining an appropriate level of gel hardness. It was found that gel hardness, spreadability, and adhesiveness were driven by the spreadability agents and gelling polymers, and decreased with reduced loadings of the gelling polymer.

In terms of selecting a stabilizer or preservative. In of selecting a gel preservative or stabilizer, an optimal compound can function as a preservative under mildly acidic to acidic conditions. Another advantageous parameter is an ability to demonstrate acceptable potential antimicrobial activity against only Gram+/−bacteria, molds, and yeasts. While the antimicrobial activity may be limited by the expected pH range of the topical gel platform. Selected examples include benzoic acid and sorbic acid.

As shown in FIGS. 18A, 18B, and 18C multiple dipivefrin gel formulations were evaluated for permeation.

Referring to FIG. 18A, this shows Topical Dipivefrin Gel Permeation as a Function of Dipivefrin Concentration and Tissue Type. The tested tissues were Porcine Ear vs. Buccal Tissue through 24 hours. After 120 minutes, differences in API permeation were observed between varying tissue types, with dose dependent permeation also being observed. Buccal tissue, lacking a pathway for follicular penetration, demonstrated reduced dipivefrin permeation.

Referring to FIG. 18B, this shows Topical Dipivefrin Gel Permeation as a Function of Dipivefrin Concentration and Tissue Type. Within the first 6 hours, differences in API permeation were observed for varying tissue types (e.g., lag observed for buccal tissue). Dose dependent permeation was observed between 2.5%-5% and 10% API loading, with 2.5% and 5% API performing similarly within porcine ear tissue. Buccal tissue, lacking a pathway for follicular penetration, demonstrated reduced dipivefrin permeation.

Referring to FIG. 18C, this shows Topical Dipivefrin Gel Flux as a Function of Dipivefrin Concentration and Tissue Type. Differences in API flux were observed between varying tissue types (e.g., lag observed for buccal tissue). Dose dependent flux was observed between 2.5%-5% and 10% API loading, with 2.5% and 5% API performing similarly within porcine ear tissue. Buccal tissue, lacking a pathway for follicular penetration, demonstrated delayed dipivefrin flux.

As shown in Table 1, three different types of polymeric gelling agent were evaluated in early development based on excipient types used in comparable topical platforms. Non-ionic cellulosic (hydroxypropylcellulose, hydroxyethylcellulose), cross-linked polyacrylic acid (Carbopol 971P, Carbopol 974P), and arabinogalactan (gum arabic) polymers were evaluated, with the non-ionic, cellulosic polymer hydroxypropylcellulose being selected based on observed formulation stability and mechanical properties (e.g., adhesiveness). Varying molecular weight grades of hydroxypropylcellulose were evaluated within the platform (e.g., Klucel HF, Klucel GF) to understand which molecular weight grade resulted in topical gel platforms combining desirable gelling properties at a low hydroxypropylcellulose loading. Klucel HF molecular weight grade was ultimately selected given the low hydroxypropylcellulose loading needed to produce topical gel platforms with regard to optimal gelling properties.

TABLE 1
Gelling Agents Evaluated in Topical Gel Platforms

| | | Polymer | Usage |
|---|---|---|---|
| | | molecular weight | range |
| Excipient | Chemical name | (Daltons, Da) | (% w/w) |
| Klucel HF | Hydroxypropylcellulose | 1,150,000 Da | 1-5% |
| Klucel GF | Hydroxypropylcellulose | 370,000 Da | 1-5% |
| Natrosol 250 HHF | Hydroxyethylcellulose | 1,300,000 Da | 1-5% |
| Carbopol 971P | Carbomer Homopolymer | N/A, crosslinked | 1-4% |
| | Type A | polymer | |
| Carbopol 974P | Carbomer Homopolymer | N/A, crosslinked | 1-5% |
| | Type B | polymer | |
| Gum arabic | Arabinogalactan | Approx. 250,000 Da | 1-5% |

As shown in Table 2, two different types of spreadability agents were evaluated in early development based on excipient types used in comparable topical gel platforms. Either organic fatty ester (diisopropyl adipate) or organosilicon (cyclomethicone) spreadability agents were evaluated, with diisopropyl adipate being selected based on observed formulation stability and mechanical properties (e.g., adhesiveness). When leveraging cyclomethicone as a potential spreadability agent, both organic (Polysorbate 80) and organosilicon (PEG-12 dimethicone) emulsifying agents were evaluated to maintain uniform emulsions of cyclomethicone within a gelled polymer matrix.

TABLE 2
Spreadability Agents and Associated Emulsifiers
Evaluated in Topical Gel Platforms

| | | Usage range |
|---|---|---|
| Excipient | Chemical name | (% w/w) |
| Diisopropyl adipate | Diisopropyl adipate | 0.5-16.5% |
| Cyclomethicone | Decamethylcyclopentasiloxane | 0.5-13.0% |
| Polysorbate 80 | Polyoxyethylene sorbitan | 0.1-1.0% |
| | monooleate | |
| PEG-12 dimethicone | Polyoxyethylene (12) | 0.1-3.0% |
| | dimethicone | |

As shown below, Table 3 outlines a list of potential and applied chemical solvents and permeation enhancers for use in topical gel platforms.

TABLE 3
Examples of Chemical Solvents and Permeation
Enhancers Used in Topical Applications

| Chemical category | Chemical name |
|---|---|
| Alcohol | Ethanol |
| | Benzyl alcohol |
| | Isopropyl alcohol |
| | n-Butanol |
| | Oleyl alcohol |
| | Lauryl alcohol |
| Ester | Isopropyl myristate |
| | Propylene carbonate |
| Ether | Dimethyl isosorbide |
| | Triacetin |
| Fatty esters | Dioctyl adipate |
| | Dioctyl phthalate |
| | Dioctyl sebacate |
| | Di-n-hexyl phthalate |
| Polyol | Propylene Glycol |
| | Glycerol |
| | Ethylene glycol |
| | Polyethylene glycol |
| | Diethylene glycol monoethyl ether |
| | (DEGEE, Transcutol P) |

Referring to FIG. 18D, this shows Dipivefrin Amount Permeated Through Porcine Ear Tissue (2.5, 5, and 10% Dipivefrin HCl).

Referring to FIG. 18E, this shows Dipivefrin Amount Permeated Through Porcine Ear Tissue (500 Dipivefrin HCl, variation of excipient loading).

Dose proportional increases in permeation between 2.5% and 10% API loading. No difference was observed in permeation observed across excipient reductions.

Referring to FIG. 18F, this shows Dipivefrin or Dibutepinephrine Amount Permeated Through Porcine Ear Tissue (2.5, 5, and 1000 API). With respect to Formulations 1-3, Dipivefrin or Dibutepinephrine (AQEP-09) loading ranged from 2.5-10% w/w. Similar permeation results were observed for both epinephrine prodrugs.

Formulation ID and % w/w

| | 1 | 2 | 3 | 4 | 5 | 6 |
|---|---|---|---|---|---|---|
| Dipivefrin HCl | 5.00 | 10.00 | 2.50 | 5.00 | 5.00 | 5.00 |
| Klucel HF | 2.00 | 2.00 | 2.00 | 2.00 | 2.00 | 2.00 |
| (hydroxypropyl | | | | | | |
| cellulose) | | | | | | |
| Propylene glycol | 30.00 | 27.50 | 31.25 | 38.25 | 37.14 | 15.00 |
| Transcutol P | 30.00 | 27.50 | 31.25 | 38.25 | 15.00 | 37.14 |
| (diethylene glycol | | | | | | |
| monoethyl ether) | | | | | | |
| Ethyl alcohol | 33.00 | 33.00 | 33.00 | 16.50 | 40.86 | 40.86 |
| Total | 100.0 | 100.0 | 100.0 | 100.0 | 100.0 | 100.0 |

Formulations 1-3: Dipevefrin loading ranging from 2.5-10% w/w.
Formulation 4: Reduced ethyl alcohol (50%) of original loading
Formulation 5: Reduced Transcutol P (50%) of original loading
Formulation 6: Reduced propylene glycol (50%) of original loading

### Example 20 Topical Gel Formulation Iterations and Physical Attributes

Multiple formulation iterations for compositional Design of Experiment (DOE) were evaluated for both chemical stability and physical attributes of the gel platform.

Referring to FIG. 19A, this figure shows Dipivefrin Topical Gel Brookfield Viscosity as a Function of Spindle Speed and Storage Duration. Viscosity of the topical gel platforms was measured via a Brookfield viscometer (Spindle S07). Sample 4-1-2 was evaluated approximately 10 months post manufacture, both initially following re-mixing and 24 hours post re-mixing, with similar results being obtained across multiple spindle speeds. A replicate, fresh sample (4-1-4) produced similar viscosity results compared to 4-1-2 across multiple spindle speeds.

Referring to FIG. 19B, this figure shows Dipivefrin Topical Gel Brookfield Viscosity as a Function of Spindle Speed and Dipivefrin Content. Viscosity was similarly measured for the topical gel platform with the inclusion of the spreadability agent diisopropyl adipate and as a function of dipivefrin content (2.5% vs. 10% w/w). Little difference in viscosity was observed between topical gels with differing dipivefrin content.

Referring to FIG. 20A, this shows residual adhesiveness for spread Klucel-based topical gels reduces to the untreated baseline within 15-30 minutes. Klucel-based formulations produce dried topical gels with reduced surface adhesiveness compared to Carbopol 971P-based gels.

Referring to FIG. 20B, this shows Dipivefrin Topical Gel Drying as a Function of Time and Formulation Composition. Evaporative loss of topical gel formulations components is consistent with the approximate loading of ethanol.

Referring to FIG. 20C, this shows Time-Dependent Surface Dipivefrin Recovery as a Function of Topical Gel Application Thickness. Evaluation of residual dipivefrin content on the surface of porcine ear tissue as a function of time following dipivefrin topical gel application (10.0% dipivefrin content). Samples collected from porcine ear tissues via swab recovery. Dipivefrin topical gel was applied at varying thicknesses to the porcine ear surface (15 mg/cm², 30 mg/cm², and 45 mg/cm²). In this example, the formulation contained Dipivefrin content: 10.0% (w/w). The gelling polymer was Klucel Hydroxypropylcellulose. The permeation enhancer: Transcutol P (DEGEE, diethylene glycol monoethyl ether). The spreadability agent: 10% Diisopropyl adipate. The solvents where Glycerin, Ethanol (16.5%). Downward trends in recoverable dipivefrin content observed for application thicknesses 15 mg/cm² and 30 mg/cm². No appreciable change in dipivefrin content observed for the application thicknesses of 45 mg/cm².

### Example 21 Process for Formulation

Referring to FIG. 21, an exemplary process in shown. In step 201, the mixture is prepared by selecting of the formulations is selected. In step 202, the addition of Transcutol P and Glycerin is performed in a stainless steel beaker and mixed until uniform. In step 203, the addition of citric acid, sorbic acid, and gelling polymer, Klucel® hydroxypropylcellulose was made to the preparation in 202. In step 204, the addition of dehydrated alcohol, 200 Proof, diisopropyl adipate and Vitamin E to the mixture and combined until uniform. Finally, in step 205, Addition of Dipivefrin HCl to the preparation of 204 is performed until homogenization is completed. Finally, the mixture is dispensed in step 206.

### Example 22 Protocol for Histamine Treatment

An in-life phase study was conducted approximately 5 days in duration. Day 0 was the day of fosing, and the study design was as follows. For Site 7, the test article (TA) was applied 60 minutes pre-challenge with a high dose of histamine. For Site 8, the TA was applied 120 minutes pre-challenge with a high dose of histamine-optional.

Animal dosing locations were marked prior to the test article (TA) application and intradermal injection. Treatment sites was placed 2 cm away from dorsal midline and spaced 6 cm between sites. Treatment area was marked at 2×2 cm. Dosing site will be measured at 0.5×0.5 cm treatment area with 5 pricks in total. Four on each corner and one in the center.

Referring to FIG. 22A, this shows the treatment sites in a cranial area. FIG. 22B shows the histamine injection site. The dosing site was visibly measured with a ruler or marked at 0.5×0.5 cm treatment area with 5 injections in total. Four on each corner and one in the center. Each injection contained ˜10 μL of histamine. Test article was administered topically with a gloved finder to the entire treatment area 2×2 cm for ˜60 second and allowed to sit for the designated time prior to histamine injection. Dose volume was not to exceed 30 mg/cm2 or 2.4 ml per test site.

A total of 50 ul of histamine was administered with an insulin syringe intradermally to the 0.5×0.5 cm treatment area. A total of five pricks was administered, each prick contained 10 ul of histamine. Four on each corner and one in the center of dosing site. Animals were observed, data recorded, and specimens collected as indicated below.

| | | | Injection | | | | | | |
|---|---|---|---|---|---|---|---|---|---|
| | | | Volume | | | | | | |
| | | Histamine | (total per | | | | | | |
| | # of | Dose | treatment | Site 1 | Test Site | Test Site | Test Site | Test Site | Test Site |
| Animal | Animals | (mg/ml) | site) | (Control) | 2 | 3 | 4 | 5 | 6 |
| 1 | 2 | 25 | 50 uL | Histamine | Applied | Applied | Applied | Applied | Applied ~5 |
| 2 | | | | Prick | 120 mins | 90 mins | 60 mins | 30 mins | mins post |
| 3 | 2 | 50 | 50 uL | ONLY | prior to | prior to | prior to | prior to | prick |
| 4 | | | | | prick | prick | prick | prick | |
| 5* | 1 | 25/50 | 50 uL | no | TA | TA | TA | no | TA |
| | | | | treatment + | applied | applied | applied | treatment + | Applied 5 |
| | | | | low dose | 5 mins | 60 mins | 120 mins | High | mins post + |
| | | | | histamine | post + ow | pre- | pre- | dose | High |
| | | | | | dose | challenge + | challenge + | histamine | dose |
| | | | | | Histamine | low dose | low dose | | Histamine |
| | | | | | | Histamine | Histamine − | | |
| | | | | | | | Optional | | |

*Site -7- TA applied 60 mins pre-challenge + High dose Histamine.
*Site -8- TA applied 120 mins pre-challenge + high dose Histamine − Optional.

Referring to FIG. 22C, gross photos of each test site were taken during prior to histamine injection and at 5, 10, 20 30, and 40 minutes post injection. The Draize scoring items measure relative severity of erythema (abnormal redness of the skin) and edema (swelling caused by excess fluid) each on a 5-point ordinal scale (0=absence, 1=trace, 2=mild, 3=moderate, and 4=severe).

| Score | Erythema | Edema |
|---|---|---|
| 0 | No erythema | No edema |
| 1 | Very slight (barely | Very slight (barely perceptible) |
| | perceptible) | |
| 2 | Well defined erythema | Slight edema (edges of area well |
| | | defined by definite raising) |
| 3 | Moderate to severe | Moderate edema (raised |
| | erythema | approximately 1 mm) |
| 4 | Severe (beet redness) to | Severe edema (raised more than |
| | slight eschar formation | 1 mm and extending beyond area |
| | | of exposure) |

The following data was obtained.

| Concen- | Animal ID | | Edema Scores (0-4) | | | | | | Erythema Scores (0-4) | | | | | |
|---|---|---|---|---|---|---|---|---|---|---|---|---|---|---|
| tration | (Dose | HTM Dose | | 5 | 10 | 20 | 30 | 40 | | 15 | 10 | 20 | 30 | 40 |
| HTM | Site) | time | Pre | min | min | min | min | min | Pre | min | min | min | min | min |
| NA | 1248294 (1) | Control | 0 | 0 | 0 | 0 | 0 | 0 | 0 | 2 | 2 | 2 | 1 | 0 |
| Low | 1248294 (2) | 120 min pre | 0 | 0 | 0 | 0 | 0 | 0 | 0 | 1 | 1 | 0 | 0 | 0 |
| Low | 1248294 (3) | 90 min pre | 0 | 0 | 0 | 0 | 0 | 0 | 0 | 1 | 1 | 1 | 1 | 1 |
| Low | 1248294 (4) | 60 min pre | 0 | 0 | 0 | 0 | 0 | 0 | 0 | 2 | 1 | 1 | 0 | 0 |
| Low | 1248294 (5) | 30 min pre | 0 | 0 | 0 | 0 | 0 | 0 | 0 | 1 | 1 | 1 | 1 | 0 |
| Low | 1248294 (6) | 5 min post | 0 | 0 | 0 | 0 | 0 | 0 | 0 | 2 | 1 | 1 | 0 | 0 |
| NA | 3174891 (1) | Control | 0 | 0 | 0 | 0 | 0 | 0 | 0 | 2 | 2* | 2 | 2 | 1 |
| Low | 3174891 (2) | 120 min pre | 0 | 0 | 0 | 0 | 0 | 0 | 0 | 1 | 1 | 1 | 0 | 0 |
| Low | 3174891 (3) | 90 min pre | 0 | 0 | 0 | 0 | 0 | 0 | 0 | 1 | 1 | 1 | 1 | 1 |
| Low | 3174891 (4) | 60 min pre | 0 | 0 | 0 | 0 | 0 | 0 | 0 | 2 | 1 | 1 | 1 | 0 |
| Low | 3174891 (5) | 30 min pre | 0 | 0 | 0 | 0 | 0 | 0 | 0 | 2 | 2 | 1 | 1 | 0*** |
| Low | 3174891 (6) | 5 min post | 0 | 0 | 0 | 0 | 0 | 0 | 0 | 2 | 2 | 2 | 1 | 1 |
| NA | 3174751 (1) | Control | 0 | 0 | 0 | 0 | 0 | 0 | 0 | 2 | 2 | 1 | 1 | 1 |
| High | 3174751 (2) | 120 min pre | 0 | 0 | 0 | 0 | 0 | 0 | 0 | 2 | 1 | 1 | 0 | 0 |
| High | 3174751 (3) | 90 min pre | 0 | 0 | 0 | 0 | 0 | 0 | 0 | 2 | 1 | 1 | 0** | 0 |
| High | 3174751 (4) | 60 min pre | 0 | 0 | 0 | 0 | 0 | 0 | 0 | 2 | 1 | 1 | 0 | 0 |
| High | 3174751 (5) | 30 min pre | 0 | 0 | 0 | 0 | 0 | 0 | 0 | 2 | 1 | 1 | 0 | 0 |
| High | 3174751 (6) | 5 min post | 0 | 0 | 0 | 0 | 0 | 0 | 0 | 2 | 3 | 1 | 0 | 0 |
| NA | 3174638 (1) | Control | 0 | 0 | 0 | 0 | 0 | 0 | 0 | 3 | 3 | 2 | 1 | 1 |
| High | 3174638 (2) | 120 min pre | 0 | 0 | 0 | 0 | 0 | 0 | 0 | 3 | 2 | 2 | 2 | 1 |
| High | 3174638 (3) | Control | 0 | 0 | 0 | 0 | 0 | 0 | 0 | 3 | 2 | 1 | 1 | 1 |
| High | 3174638 (4) | 60 min pre | 0 | 0 | 0 | 0 | 0 | 0 | 0 | 2 | 2 | 1 | 1 | 0 |
| High | 3174638 (5) | 30 min pre | 0 | 0 | 0 | 0 | 0 | 0 | 0 | 3 | 2 | 2 | 1 | 0 |
| High | 3174638 (6) | 5 min post | 0 | 0 | 0 | 0 | 0 | 0 | 0 | 3 | 3 | 2 | 2 | 1 |
| Low | 1249266 (1) | Control | 0 | 0 | 0 | 0 | 0 | 0 | 0 | 2 | 2 | 1 | 1 | 1 |
| Low | 1249266 (2) | 5 min post | 0 | 0 | 0 | 0 | 0 | 0 | 0 | 2 | 2 | 2 | 1 | 0 |
| Low | 1249266 (3) | 60 min pre | 0 | 0 | 0 | 0 | 0 | 0 | 0 | 2 | 2 | 2 | 1 | 0 |
| High | 1249266 (5) | Control | 0 | 0 | 0 | 0 | 0 | 0 | 0 | 2 | 2 | 1 | 1 | 1 |
| High | 1249266 (6) | 5 min post | 0 | 0 | 0 | 0 | 0 | 0 | 0 | 2 | 2 | 2 | 1 | 0 |
| High | 1249266 (7) | 60 min pre | 0 | 0 | 0 | 0 | 0 | 0 | 0 | 2 | 2 | 1 | 1 | 1 |

*Bleeding noted post injection. Blood removed with water-soaked gauze prior to 10 minute scoring and photo to better visualize erythem
**Blood from injection partially obscurring test site
***Small abrasion noted in upper right corner of test site reported to Study Director

### Example 23 Systemic Pharmacokinetic Characteristics with Skin Abrasion

A study was conducted to evaluate the systemic pharmacokinetic characteristics of Dipivefrin Topical Gel when administered once by topical application to intact, abraded, and wounded skin of Sinclair Nanopigs™. The study design was as follows.

Animals were food fasted for at least 8 hours. Animals were monitored until fully recovered. Anesthesia procedures were documented and retained in the study data. The dose formulations were administered to appropriate animals by topical application on the dorsal surface (Dose Site #1) once on Day 1. The dose site was and/or shaved and marked at least one day prior to Day 1. At least the four corners of the dose sites will be marked with indelible ink based on the appropriate dose area. At least the four corners of the tape stripping area were marked with indelible ink for Group 2 and 3 animals. The site for Group 2 and 3 animals was gently abraded via tape stripping (conducted 50 times).

All animals will have a uniform topical application using a gloved hand and/or spatula to rub the entire dose amount throughout the total dose area (for 1 minute). Topical application to the dose area for Group 4 animals will include application of the dose amount over the created wounds. If an animal is observed to have test material remaining following the application time, the observation will be documented.

Dose formulations will be allowed to equilibrate at ambient temperature for at least 15 minutes prior to and continuously throughout dose administration

Prior to dermal (topical) application on Day 1, the dose site for Group 1 to 3 animals were wiped clean with water-moistened gauze or paper towels then wiped dry with dry gauze or paper towels. Following washing and drying, a 7×7 cm area within the dose.

On Day 1, Group 4 animals: The animal backs were prepared for wound creation by performing a surgical scrub. Two (2) wounds were created on the dorsal surface of each Group 4 animal within a 7×7 cm area using an 8 mm punch biopsy. Steps were taken to ensure the area designated for wound creation is within the total dose area (8×8 cm). A fresh 8 mm biopsy punch was used for each wound. Suturing and/or bandaging of wounds were not conducted

On Day 2, Male No. 1001 and Group 2 animals: The animal backs were prepared for in-life biopsies by performing a surgical scrub. Two (2) 5 mm punch biopsies were collected from within a 7×7 cm area. Steps were taken to ensure the area designated for in-life biopsy collection is within the total dose area (8×8 cm). A fresh 5 mm biopsy punch will be used for each collection. Suturing and/or bandaging of wounds may be conducted, as needed. Samples will be fixed in neutral buffered formalin (NBF); 1 sample will be processed for histopathology and 1 sample will be stored in neutral buffered formalin (NBF) for potential future analysis.

The results were as follows:

| | | | | Total | | | |
|---|---|---|---|---|---|---|---|
| | | Dose | Dose | Dose | Individual | | |
| | Test | Conc. | Amount | Area | Dose Area | Dose | Number |
| Group | Material | (%) | (mg/cm²) | (cm²) | Dimensions | Route | of Males |
| 1 | Dipivefrin | 2.5 | 30 | 64 | 8 × 8 cm | Topical | 3 |
| | Topical | | | | | (Intact | |
| | Gel | | | | | Skin) | |
| 2 | Dipivefrin | 2.5 | 30 | 128 | 8 × 8 cm | Topical | 3 |
| | Topical | | | | (x2) | (Abraded | |
| | Gel | | | | | Skin, | |
| | | | | | | 50 tape | |
| | | | | | | strips) | |
| 3 | Dipivefrin | 2.5 | 30 | 256 | 8 × 8 cm | Topical | 3 |
| | Topical | | | | (x4) | (Abraded | |
| | Gel | | | | | Skin, | |
| | | | | | | 50 tape | |
| | | | | | | strips) | |
| 4 | Dipivefrin | 2.5 | 30 | 64 | 8 × 8 cm | Topical | 3 |
| | Topical | | | | | (Wounded | |
| | Gel | | | | | Skin) | |

Referring to FIG. 23A, this shows the measured dipivefrin plasma concentration over time, comparing intact skin, abraded skin (128 cm²), abraded skin (256 cm²) and wounded skin (64 cm²). Mean Cmax values were higher (>2-fold) in animals with wounded skin versus intact skin. The wounded to intact skin ratio for Cmax was 4.60.

Referring to FIG. 23B, this shows the measured epinephrine plasma concentration over time for intact skin, abraded skin (128 cm²), abraded skin (256 cm²) and wounded skin (64 cm²). Mean Cmax, AUClast, and AUC0-6 values were generally higher (approaching or >2-fold) in animals with wounded skin versus intact skin. The wounded to intact skin ratios were 3.90 for Cmax

As indicated in FIGS. 23A and 23B and data below, very few quantifiable dipivefrin concentrations were observed. Tmax and Cmax were shown to be higher in the group with full thickness wound. There was no significant difference in PK parameters in the plasma between intact skin and abraded skin, for both dipivefrin and epinephrine.

| | Dose | Dose | Total | | Tmax | Cmax | AUClast | AUC0-6 | Tmax | Cmax | AUClast | AUC0-6 |
|---|---|---|---|---|---|---|---|---|---|---|---|---|
| | Amount | Area | Dose | Dose | (h) | (ng/mL) | (h*ng/mL) | (h*ng/mL) | (h) | (ng/mL) | (h*ng/mL) | (h*ng/mL) |
| Group | (mg/cm2) | (mg/cm2) | (mg) | Route | Dipivefrin | | | | Epinephrine | | | |
| 1 | 30 | 64 | 1920 | Topical | 1.00 | 0.402 | NR | NR | 2.00 | 1.95 | 7.62 | 8.07 |
| | | | | (Intact | | | | | | | | |
| | | | | Skin) | | | | | | | | |
| 2 | 30 | 128 | 3840 | Topical | 1.50 | 10.375 | NR | NR | NR | NR | NR | NR |
| | | | | (Abraded | | | | | | | | |
| | | | | Skin) | | | | | | | | |
| 3 | 30 | 256 | 7680 | Topical | 1.00 | 0.193 | NR | NR | 3.00 | 1.47 | 6.33 | 6.33 |
| | | | | (Abraded | | | | | | | | |
| | | | | Skin) | | | | | | | | |
| 4 | 30 | 64 | 1920 | Topical | 0.670 | 1.85 | 1.50 | 2.07 | 2.00 | 7.60 | 14.0 | 15.6 |
| | | | | (Wounded | | | | | | | | |
| | | | | Skin) | | | | | | | | |

### Example 24 Effect of Epinephrine and Dipivefrin on NK Activation

A study was conducted to evaluate the effect of epinephrine and dipivefrin on NIK activation and cytotoxicity. Peripheral blood mononuclear cells (PBMC) were isolated over a density gradient from six donor buffy coats and CD56+NK were separated using magnetic selection. Purity of sorted NIK cells was assessed by flow cytometry using CD56 as a marker of NK cells.

### NIK Activation Assay

Following magnetic isolation, NK cells were seeded at 5×10⁵ NK cells per well in a 48 well plate alongside NK activation beads (prepared as per manufacturer's protocol) at a 1:1 bead to cell ratio, IL-2 (final concentration 500 IU/ml) and either prodrug/epinephrine at 5 different doses (see section 3.6 below for further details), cortisol/dexamethasone (positive controls) or vehicle (media only). NK cells were cultured with the NK activation beads for two days at 37° C. in the presence of 5% CO2. After two days, supernatants were collected from each condition for future TR-FRET analysis (AQV-24-001_AM-1 only, see below) and the remaining cells from each condition were assessed for expression of surface markers CD25, CD69 and for cell viability using eBioscience™ Fixable Viability Dye. Samples were then treated with eBioscience Intracellular Fixation & Permeabilization Buffer Set to allow staining of intracellular granzyme B. Marker expression was determined by flow cytometry and appropriate “Fluorescence-Minus-One controls” controls were used. Flow cytometry data was analysed using FlowJo analysis software and data was graphed using Graph Pad Prism.

As shown in FIGS. 24A, 24B, 24C, 24D, 24E and 24F, Treatment of NK cells with prodrug resulted in consistent reduction in CD25⁺CD69⁺ NK cells compared to the vehicle control. Donor 1 removed from the data as an outlier. Lower doses of prodrug, tested in donors 2-3, led to a 7-12% reduction in CD25⁺CD69⁺ NK cells and higher doses of prodrug, tested in donors 4-6, led to a comparable 5-12% reduction in CD25⁺CD69⁺ NK cells. Although we see about 12% reduction of NK cell activation as compared to control, it is comparable to the effect of 20 nM Cortisol. Higher doses of prodrug, up to 2 uM, are being tested to verify if dose dependency is achieved at significantly higher doses than 5 ng/mL tested here.

As shown in FIGS. 25A, 25B, 25C, 25D, 25E and 25F, Treatment of NK cells with Epinephrine resulted in consistent reduction in CD25⁺CD69⁺ NK cells, similar to that observed with Dipivefrin. Donor 1 removed from the data as an outlier. For 5 out of 6 of the donors, the proportion of CD25⁺CD69⁺ NK cells decreased with increasing doses of epinephrine. When compared at the similar doses, prodrug and epinephrine showed a similar efficacy in inhibiting NK cells, as there was a comparable proportion of CD25⁺CD69⁺ NK cells in prodrug- and epinephrine-treated donors

### Example 25 IFNγ TR-FRET Assay

Supernatants collected from the NK activation assay above were stored at −80° C. for further analysis. Concentrations of IFNγ in the supernatants were subsequently assessed by TR-FRET, using the manufacturer's protocol for this assay. Standards of known concentration were run on each plate and a standard curve was generated using MARS software to apply a four-parameter logistic fit to the data. Values not quantified due to falling below the Lower Limit of Quantification (LLOQ) were not plotted.

### Donor 1-3 (AQV-24-001-2) Conditions Table

The study was performed in: 3 donors Each experimental condition was tested in: singlicate. For Flow cytometry, viability was measured and cells were phenotyped for NK cell activation (CD25, CD69, granzyme B). Raw and analysed data are provided.

| Condition | Dose | Stimulation | Readouts |
|---|---|---|---|
| Vehicle | N/A | NK | Flow |
| Prodrug | 0.01 μg/ml, 0.1 μg/ml, | activation | cytometry |
| | 0.5 μg/ml, 1 μg/ml, 5 μg/ml | beads and | (day 2): |
| Epinephrine | 0.01 μg/ml, 0.05 μg/ml, | IL-2 | Viability, |
| | 0.1 μg/ml,0.25 μg/ml, | | CD25, CD69, |
| | 0.5 μg/ml | | granzyme B |
| Cortisol | 200 nM | | |

### Donor 4-6 (AQV-24-001-2-AM1) Conditions Table

The study was performed in: 3 donors.

Each experimental condition was tested in: singlicate. For Flow cytometry, viability was measured and cells were phenotyped for NK cell activation (CD25, CD69, granzyme B). Raw and analysed data are provided. Supernatants were harvested in triplicate per condition and stored until further analysis of IFNγ content by TR-FRET. Data was analysed and graphed. Raw and analysed data are provided.

| Condition | Dose | Stimulation | Readouts |
|---|---|---|---|
| Vehicle | N/A | NK | Flow |
| Prodrug | 5 μg/ml, 10 μg/ml, | activation | cytometry |
| | 25 μg/ml, 50 μg/ml, | beads and | (day 2): |
| | 100 μg/ml″ | IL-2 | Viability, |
| Epinephrine | 0.5 μg/ml, 1 μg/ml, | | CD25, CD69, |
| | 5 μg/ml, 10 μg/ml, | | granzyme B |
| | 50 μg/ml | | |
| Cortisol | 200 nM, 20 nM | | |
| Dexamethasone | 1 μM | | |

### Example 26 NK Deactivation

As shown in FIG. 26A, when the effect of Epinephrine and Dipivefrin is combined for analysis, an effective suppression of activation of NK cells was observed across a range of concentrations, exceeding the IC50 at 70, 140, and 280 nM concentrations.

As shown in FIGS. 26B, 26C, 26D, 26E, 26F and 26G, there was also a noticeable effect on the Median Fluorescence Intensity (MFI) of Granzyme B, with expression of this marker decreasing to some extent in donors 4-6 at concentrations of prodrug from 100 μg/ml as compared to the vehicle control. GrB expression is strictly regulated by Jak, and oral Jak inhibitors effectively reduce the mRNA expression of GrB in the skin of the treated patients. Topical strategies targeting local GrB production in the skin might be safer and more specific treatment options in AA. The Possible Linkage of Granzyme B-Producing Skin T Cells with the Disease Prognosis of Alopecia Areata Koguchi-Yoshioka, Hanako et al. Journal of Investigative Dermatology, Volume 141, Issue 2, 427-429.e10.

CD56+ cells were isolated from three donor buffy coats by magnetic separation. Purity of CD56+ cells following magnetic isolation was 85.4, 78.7 and 85% for Donor 1, 2 and 3, respectively. Next, the effect of compound addition at different timepoints and doses, following either optimal or suboptimal NK activation was determined by measuring expression of the activation markers CD25, CD69 and Granzyme B on NK cells. NK cells displayed the expected activation profile with a greater proportion of NK cells being CD25+CD69+ at 48 h compared to 24 h. There were however, donor dependent differences with the greatest response observed in Donor 1, around 60% at 24 h and 80% at 48 h. Optimal stimulation for the most part, provided a larger therapeutic window compared to sub-optimal stimulation for both CD25+CD69+ and Granzyme B readouts. Treatment was administered at 0, 6, 24 and 30 hours. The controls were 200 nM Cortisol and 500 nM Deuruxolitinib (Jak inhibitor).

Referring to FIGS. 26H and 26I, this shows the effect of prodrug and control compounds on proportion of CD25⁺CD69⁺NK cells when administered at 0 and 6 hours (Fold change).

Referring to FIGS. 26J and 26K, this shows the effect of epinephrine and control compounds on proportion of CD25+CD69+NK cells when administered at 0, 6, 24, and 30 hours (Fold change).

Other embodiments are within the scope of the following claims.

What is claimed is:

1. A topical pharmaceutical composition, comprising:
a pharmaceutically active component including at least one prodrug of epinephrine; and
a skin permeability enhancer.

2. The pharmaceutical composition of claim 1 further comprising a gelling polymer.

3. The pharmaceutical composition of claim 1 further comprising a solvent.

4. The pharmaceutical composition of claim 1 further comprising a spreadability agent.

5. The pharmaceutical composition of claim 1 further comprising an antioxidant.

6. The pharmaceutical composition of claim 1 further comprising a preservative.

7. The pharmaceutical composition of claim 1 further comprising a pH modifier.

8. The pharmaceutical composition of claim 1 further comprising a viscosity agent.

9. The pharmaceutical composition of claim 2 wherein the gelling polymer is a non-ionic cellulosic polymer.

10. The pharmaceutical composition of claim 2 wherein the gelling polymer is a cross-linked polyacrylic acid.

11. The pharmaceutical composition of claim 2 wherein the gelling polymer is a arabinogalactan or gum arabic polymer.

12. The pharmaceutical composition according to claim 1, further comprising a permeation enhancer.

13. The pharmaceutical composition according to claim 1, wherein the topical composition is formed from a gauze, hydrogel, ampule, solution, paste, a cream, a lotion, a powder, emulsion, an ointment, a gel, a patch, liquid or spray.

14. The pharmaceutical composition according to claim 1, wherein the spray is formed in an enclosure over a treatment area.

15. The pharmaceutical composition according to claim 1, wherein the skin permeability enhancer is a solvent and solubilizer.

16. The pharmaceutical composition according to claim 1, wherein the skin permeability enhancer can be an ether, such as a monoethyl ether.

17. The pharmaceutical composition according to claim 1, wherein the skin permeability enhancer is diethylene glycol monoethyl ether.

18. The pharmaceutical composition according to claim 1, wherein the topical pharmaceutical composition includes an adrenergic receptor interacter.

19. The pharmaceutical composition according to claim 8, wherein the adrenergic receptor interacter is eugenol or eugenol acetate, a cinnamic acid, cinnamic acid ester, cinnamic aldehyde, hydrocinnamic acid, chavicol, or safrole.

20. The pharmaceutical composition according to claim 1, wherein the adrenergic receptor interacter is a phytoextract.

21. The pharmaceutical composition according to claim 10, wherein the phytoextract further includes an essential oil extract of a clove plant, an essential oil extract of a leaf of a clove plant, an essential oil extract of a flower bud of a clove plant, or an essential oil extract of a stem of a clove plant.

22. The pharmaceutical composition according to claim 1, further comprising a mixed ester, such as a cellulose or a modified cellulose.

23. The pharmaceutical composition according to claim 13, wherein the mixed ester is synthetic or biosynthetic.

24. The pharmaceutical composition according to claim 1, wherein the pharmaceutical composition includes a cellulosic polymer is selected from the group of: hydroxypropylmethyl cellulose, hydroxyethyl cellulose, hydroxyethylmethyl cellulose, hydroxypropyl cellulose, methylcellulose and carboxymethyl cellulose.

25. The pharmaceutical composition according to claim 1, further comprising a stabilizer.

26. A method of making a topical pharmaceutical composition comprising:
combining a pharmaceutically active component including at least one prodrug of epinephrine and a skin permeability enhancer, and
forming a pharmaceutical composition including the skin permeability enhancer and the pharmaceutically active component.

27. A method of treating a medical condition comprising:
administering an effective amount of a topical pharmaceutical composition comprising a pharmaceutically active component including at least one prodrug of epinephrine; and a skin permeability enhancer.

28. The method of claim 17, wherein the prodrug is an ester of epinephrine.

29. The method of claim 17, wherein the medical condition includes alopecia, contact hypersensitivity, aging skin, pemphigus, psoriasis, pruritis, atopic dermatitis, wounds, melanoma, vitiligo, alopecia, acne, alopecia areata, Raynaud's phenomenon, epidermolysis bullosa, rosacea, scleroderma, hidradenitis suppurativa (acne inversa), ichthyosis, pachyonychia congenital, or urticaria.

30. The topical pharmaceutical composition according to claim 18, wherein the prodrug is an ester of epinephrine, such as dipifevrin.

31. The topical pharmaceutical composition according to claim 1, wherein epinephrine or its prodrug impact mast cells or histamines.

32. The topical pharmaceutical composition according to claim 1 wherein epinephrine or its prodrug target disease in which natural killer (NK) cells are activated.

33. The topical pharmaceutical composition according to claim 1 wherein epinephrine or its prodrug target act as immunosuppressants.

34. The topical pharmaceutical composition according to claim 1 wherein epinephrine or its prodrug act as melanogenic agents.

35. The topical pharmaceutical composition according to claim 1 wherein epinephrine or its prodrug inhibits cytokine production.

36. The topical pharmaceutical composition according to claim 1 wherein epinephrine or its prodrug elevates TNF-α.

37. The topical pharmaceutical composition according to claim 1 wherein epinephrine or its prodrug elevates IFNΥ.
